SEQUENCE LISTING (26 sequences)

SEQ ID NO: 1 - protein, 241 aa, Artificial Sequence
  SLAMF7-binding element; huLuc63 VH, linker, huLuc63 VL
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGGGGSGGGGSGGGGSDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 2 - protein, 228 aa, Artificial Sequence
  IgG4-FC spacer domain; hinge, CH2, CH3 with 4/2 NQ modification
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 28 aa, Artificial Sequence
  CD28 transmembrane domain
MFWVLVVVGGVLACYSLLVTVAFIIFWV

SEQ ID NO: 4 - protein, 41 aa, Artificial Sequence
  CD28 cytoplasmic domain
RSKRSRGGHSDYMNMTPRRPGPTRKHYQPYAPPRDFAAYRS

SEQ ID NO: 5 - protein, 112 aa, Artificial Sequence
  CD3 zeta domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 469 aa, Artificial Sequence
  SLAMF7-binding CAR, extracellular domain; huLuc63 VH, linker, huLuc63 VL, hinge, CH2, CH3
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGGGGSGGGGSGGGGSDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 153 aa, Artificial Sequence
  SLAMF7-binding CAR, intracellular signalling domain; CD28 cytoplasmic, CD3 zeta
RSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 650 aa, Artificial Sequence
  SLAMF7-binding CAR; huLuc63 VH, linker, huLuc63 VL, hinge, CH2, CH3, CD28 tm, CD28 cytoplasmic, CD3 zeta
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGGGGSGGGGSGGGGSDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 225 bp, Artificial Sequence
  left IR/DR segment; left inverted repeats which are recognized and bound by transposase
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacact

SEQ ID NO: 10 - DNA, 225 bp, Artificial Sequence
  right IR/DR segment; right inverted repeats which are recognized and bound by transposase
agtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 11 - DNA, 4352 bp, Artificial Sequence
  SLAMF7-binding CAR integration cassette comprising left IR/DR, EF-1 alpha core promoter, Kozak, GMCSF SP, huLuc63 VH, (4GS)3 linker, huLuc63 VL, IgG4 Hinge, IgG4 CH2CH3 NQ, CD28 tm, CD28 cytoplasmic, T2A, EGFRt, right IR/DR
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttgatatccatggaattcggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgctgctgctcgtgacatctctgctgctgtgcgagctgccccaccccgcctttctgctgattcctgaggtgcagctggtggaaagcggcggaggactggtgcagcctggcggatctctgagactgagctgtgccgccagcggcttcgacttcagccggtactggatgagctgggtgcgccaggcccctggcaaaggcctggaatggatcggcgagatcaaccccgacagcagcaccatcaactacgcccccagcctgaaggacaagttcatcatcagccgggacaacgccaagaacagcctgtacctgcagatgaactccctgcgggccgaggacaccgccgtgtactattgcgccagacccgacggcaactactggtacttcgacgtgtggggccagggcaccctcgtgacagtgtctagcggaggcggcggatctggcggagggggatctgggggcggaggctctgatatccagatgacccagagccccagcagcctgtctgccagcgtgggcgacagagtgaccatcacatgcaaggccagccaggacgtgggaatcgccgtggcctggtatcagcagaaacccggcaaggtgcccaagctgctgatctactgggccagcaccagacacaccggcgtgcccgatagattttccggcagcggctccggcaccgacttcaccctgacaatcagctccctgcagcctgaggacgtggccacctactactgccagcagtacagcagctacccctacaccttcggacagggcaccaaggtggaaatcaaagagtctaagtacggaccgccttgtcctccttgtccagctcctcctgtggccggacctagcgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttccagagcacctaccgggtggtgtccgtgctgacagtgctgcaccaggactggctgaacggcaaagagtacaagtgcaaggtgtccaacaagggcctgcccagcagcatcgagaaaaccatcagcaaggccaagggccagcctcgcgagccccaggtgtacacactgcctccaagccaggaagagatgaccaagaaccaggtgtccctgacctgtctcgtgaagggcttctaccccagcgacattgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaagatgttctgggtgctggtggtcgtgggcggagtgctggcctgttacagcctgctcgtgaccgtggccttcatcatcttttgggtgcgcagcaagcggagcagaggcggccacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggataagcggagaggccgggaccctgagatgggcggcaagcctagaagaaagaacccccaggaaggcctgtataacgaactgcagaaagacaagatggccgaggcctacagcgagatcggcatgaagggcgaaagaaggcggggcaagggccacgatggcctgtatcagggactgagcaccgccaccaaggatacctatgacgcactgcacatgcaggccctgccccccagactcgagggcggaggcgaaggcagaggatctctgctgacatgcggcgacgtggaagagaacccaggccccagaatgctgctgctcgtgacaagcctgctgctgtgcgagctgccccaccctgcctttctgctgatcccccggaaagtgtgcaacggcatcggcatcggagagttcaaggacagcctgtccatcaacgccaccaacatcaagcacttcaagaattgcaccagcatcagcggcgacctgcacatcctgccagtggcctttagaggcgacagcttcacccacacccccccactggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagatggccagatctagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 12 - DNA, 4611 bp, Artificial Sequence
  SLAMF7 CAR minicircle DNA
gccttggtcaaattgggtatacccatttgggcctagtctagccggcatggcgcattacagcaatacgcaatttaaatgcgcctagcgcattttcccgaccttaatgcgcctcgcgctgtagcctcacgcccacatatgtgcagatctgaagaagaagaagaagaagaagaagaagaagaagaagaagaagaagaagaagaagaagaagaagtttctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagcttgatatccatggaattcggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgctgctgctcgtgacatctctgctgctgtgcgagctgccccaccccgcctttctgctgattcctgaggtgcagctggtggaaagcggcggaggactggtgcagcctggcggatctctgagactgagctgtgccgccagcggcttcgacttcagccggtactggatgagctgggtgcgccaggcccctggcaaaggcctggaatggatcggcgagatcaaccccgacagcagcaccatcaactacgcccccagcctgaaggacaagttcatcatcagccgggacaacgccaagaacagcctgtacctgcagatgaactccctgcgggccgaggacaccgccgtgtactattgcgccagacccgacggcaactactggtacttcgacgtgtggggccagggcaccctcgtgacagtgtctagcggaggcggcggatctggcggagggggatctgggggcggaggctctgatatccagatgacccagagccccagcagcctgtctgccagcgtgggcgacagagtgaccatcacatgcaaggccagccaggacgtgggaatcgccgtggcctggtatcagcagaaacccggcaaggtgcccaagctgctgatctactgggccagcaccagacacaccggcgtgcccgatagattttccggcagcggctccggcaccgacttcaccctgacaatcagctccctgcagcctgaggacgtggccacctactactgccagcagtacagcagctacccctacaccttcggacagggcaccaaggtggaaatcaaagagtctaagtacggaccgccttgtcctccttgtccagctcctcctgtggccggacctagcgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttccagagcacctaccgggtggtgtccgtgctgacagtgctgcaccaggactggctgaacggcaaagagtacaagtgcaaggtgtccaacaagggcctgcccagcagcatcgagaaaaccatcagcaaggccaagggccagcctcgcgagccccaggtgtacacactgcctccaagccaggaagagatgaccaagaaccaggtgtccctgacctgtctcgtgaagggcttctaccccagcgacattgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaagatgttctgggtgctggtggtcgtgggcggagtgctggcctgttacagcctgctcgtgaccgtggccttcatcatcttttgggtgcgcagcaagcggagcagaggcggccacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggataagcggagaggccgggaccctgagatgggcggcaagcctagaagaaagaacccccaggaaggcctgtataacgaactgcagaaagacaagatggccgaggcctacagcgagatcggcatgaagggcgaaagaaggcggggcaagggccacgatggcctgtatcagggactgagcaccgccaccaaggatacctatgacgcactgcacatgcaggccctgccccccagactcgagggcggaggcgaaggcagaggatctctgctgacatgcggcgacgtggaagagaacccaggccccagaatgctgctgctcgtgacaagcctgctgctgtgcgagctgccccaccctgcctttctgctgatcccccggaaagtgtgcaacggcatcggcatcggagagttcaaggacagcctgtccatcaacgccaccaacatcaagcacttcaagaattgcaccagcatcagcggcgacctgcacatcctgccagtggcctttagaggcgacagcttcacccacacccccccactggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagatggccagatctagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgactgca

SEQ ID NO: 13 - protein, 340 aa, Artificial Sequence
  SB transposase
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 14 - DNA, 6637 bp, Artificial Sequence
  pcGlobin2-SB100X plasmid
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggcacgagctggactttaagcaagcacctaaggacatccaaaagcgtaaacggatccaccatgggcaaatccaaggagatctcccaggacctgaggaaaagaatcgtggacctgcacaaatctggctcttctctgggcgccatctctaaaagactggccgtgcctaggtccagcgtgcagaccattgtgcggaaatacaaacaccacggaaccacacagccatcttaccgctccggacggcggagagtgctgtcccctagagacgagaggaccctcgtgagaaaagtgcagatcaaccctagaacaaccgccaaagacctggtgaaaatgctggaggagaccggcaccaaggtgtccatctctaccgtgaagcgcgtgctgtaccggcacaacctgaagggacactccgcccggaagaaacctctgctgcagaataggcacaagaaagcccggctgagattcgccaccgcccacggcgataaggatagaaccttttggcgcaacgtgctgtggagcgacgagacaaaaatcgagctgttcgggcacaacgatcaccgctacgtgtggcgcaaaaagggagaggcctgtaagccaaaaaacaccatccccaccgtgaaacacggcggaggctccattatgctgtggggctgctttgccgccggcggaaccggagccctgcacaaaatcgacggcatcatggatgccgtgcagtacgtggacatcctgaaacagcacctgaaaacctctgtgagaaaactgaaactgggccgcaaatgggtgttccagcacgacaatgaccctaagcacacatccaaagtggtggccaaatggctgaaagacaacaaagtgaaagtgctggagtggccttcccagtcccccgatctgaacccaattgagaacctgtgggccgagctgaagaaaagagtgcgggccagacggcctacaaacctgacacagctgcaccagctgtgtcaggaggagtgggccaaaatccaccccaactactgtggcaagctggtggagggatacccaaaacggctgacccaagtgaaacagttcaagggcaacgccaccaagtactgagaattctgcagatatccatcacactggcggccgctcgagtctcatcgccaatgaacgtcaggtgttttggagaaggtcacaccgcgcggagatgttcaaacaaagatctaactcttttgcaacaagatcaataaaataatgttaaaagcaaaaaaaaaaaaaaaaaaaaaaaaaaaaaatctagaatcgatgcgcgctagcccgggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 15 - protein, 335 aa, Artificial Sequence
  EGFRt; truncated EGFR tag to facilitate testing for CAR+ cells and as potential depletion marker (suicide switch)
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 16 - DNA, 1008 bp, Artificial Sequence
  EGFRt; truncated EGFR tag to facilitate testing for CAR+ cells and as potential depletion marker (suicide switch)
cggaaagtgtgcaacggcatcggcatcggagagttcaaggacagcctgtccatcaacgccaccaacatcaagcacttcaagaattgcaccagcatcagcggcgacctgcacatcctgccagtggcctttagaggcgacagcttcacccacacccccccactggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 17 - protein, 119 aa, Artificial Sequence
  huLuc63 VH heavy chain of variable fragment derived from SLAMF7-binding antibody huLuc63
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 18 - DNA, 357 bp, Artificial Sequence
  huLuc63 VH heavy chain of variable fragment derived from SLAMF7-binding antibody huLuc63
gaggtgcagctggtggaaagcggcggaggactggtgcagcctggcggatctctgagactgagctgtgccgccagcggcttcgacttcagccggtactggatgagctgggtgcgccaggcccctggcaaaggcctggaatggatcggcgagatcaaccccgacagcagcaccatcaactacgcccccagcctgaaggacaagttcatcatcagccgggacaacgccaagaacagcctgtacctgcagatgaactccctgcgggccgaggacaccgccgtgtactattgcgccagacccgacggcaactactggtacttcgacgtgtggggccagggcaccctcgtgacagtgtctagc

SEQ ID NO: 19 - protein, 107 aa, Artificial Sequence
  huLuc63 VL light chain of variable fragment derived from SLAMF7-binding antibody huLuc63
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 20 - DNA, 321 bp, Artificial Sequence
  huLuc63 VL light chain of variable fragment derived from SLAMF7-binding antibody huLuc63
gatatccagatgacccagagccccagcagcctgtctgccagcgtgggcgacagagtgaccatcacatgcaaggccagccaggacgtgggaatcgccgtggcctggtatcagcagaaacccggcaaggtgcccaagctgctgatctactgggccagcaccagacacaccggcgtgcccgatagattttccggcagcggctccggcaccgacttcaccctgacaatcagctccctgcagcctgaggacgtggccacctactactgccagcagtacagcagctacccctacaccttcggacagggcaccaaggtggaaatcaaa

SEQ ID NO: 21 - protein, 22 aa, Artificial Sequence
  signal peptide 1
MLLLVTSLLLCELPHPAFLLIP

SEQ ID NO: 22 - protein, 15 aa, Artificial Sequence
  linker
GGGGSGGGGSGGGGS

SEQ ID NO: 23 - protein, 25 aa, Artificial Sequence
  T2A sequence
LEGGGEGRGSLLTCGDVEENPGPRM

SEQ ID NO: 24 - protein, 21 aa, Artificial Sequence
  signal peptide 2
LLLVTSLLLCELPHPAFLLIP

SEQ ID NO: 25 - protein, 42 aa, Artificial Sequence
  4-1BB domain
KRGRKKLLYIFKQPFMRPVQTTQEEDGCSCRFPEEEEGGCEL

SEQ ID NO: 26 - DNA, 126 bp, Artificial Sequence
  4-1BB domain
aagcggggcagaaagaagctgctgtatatcttcaagcagcccttcatgcggcccgtgcagaccacacaggaagaggacggctgctcctgccggttccccgaggaagaagaaggcggctgcgagctg

(19) United States
(12) Patent Application Publication
(10) Pub. No.: US 2023/0242641 A1
(43) Pub. Date: Aug. 3, 2023

(54) SLAMF7 CARS

(71) Applicant: JULIUS-MAXIMILIANS-UNIVERSITÄT WÜRZBURG, Würzburg (DE)

(72) Inventors: Michael HUDECEK, Höchberg (DE); Sabrina PROMMERSBERGER, Rimpar (DE)

(21) Appl. No.: 18/010,801

(22) PCT Filed: Jun. 29, 2021
(86) PCT No.: PCT/EP2021/067819

§ 371 Date: Dec. 15, 2022

(30) Foreign Application Priority Data
Jun. 29, 2020 (EP) 20182866.2

(51) Int. Cl. C07K 16/28; C07K 14/705; C07K 14/725; A61P 35/00; A61K 39/00; C07K 14/535; C07K 14/71; C12N 5/0783

(52) U.S. Cl. CPC C07K 16/2803; C07K 14/70521; C07K 14/7051; C07K 14/70578; A61P 35/00; A61K 39/4631; A61K 39/4611; C07K 14/535; A61K 39/464411; C07K 14/71; C12N 5/0636; A61K 2039/505; C07K 2319/33; A61K 38/00

(57) ABSTRACT

The present invention relates to a polypeptide encoding a SLAMF7-binding chimeric antigen receptor (CAR), a polynucleotide encoding the SLAMF7-binding CAR polypeptide, a recombinant immune cell (preferably recombinant lymphocyte, more preferably recombinant T cell) comprising the polynucleotide, a method for producing recombinant immune cells and a pharmaceutical composition comprising recombinant immune cells. The recombinant immune cells and the pharmaceutical composition of the present invention may be used in methods for treating cancer in a patient thereby providing an improved treatment regimen. The inventors of the present application demonstrated that SLAMF7 CAR T-cells prepared by Sleeping Beauty gene transfer confer superior anti-myeloma efficacy in vivo compared to SLAMF7 CAR T-cells prepared by lentiviral gene transfer. Hence, SLAMF7 CAR T-cells that are prepared by virus-free SB gene transfer possess greater anti-myeloma efficacy and therapeutic potential, which leads to significantly improved clinical activity, and significantly improved clinical outcome.

### FIELD OF INVENTION

The present invention relates to a polypeptide encoding a SLAMF7-binding chimeric antigen receptor (CAR), a polynucleotide encoding the SLAMF7-binding CAR polypeptide, a recombinant immune cell comprising the polynucleotide, a method for producing recombinant immune cells and a pharmaceutical composition comprising recombinant immune cells. The recombinant immune cells and the pharmaceutical composition of the present invention may be used in methods for treating a disease in a patient.

### BACKGROUND

Multiple myeloma (MM) is a hematological malignancy resulting from the uncontrolled proliferation of plasma cells, which leads to production of excess immunoglobulin and is associated with immunosuppression, myelosuppression and end-organ damage. MM is an incurable disease and accounts for 10% of all hematological malignancies. In the European Union (EU), 4.5 to 6 per 100,000 subjects have been diagnosed per year with a median age between 65 and 70 years. The mortality rate is 4.1/100,000 subjects per year.

Almost all patients with MM evolve from an asymptomatic premalignant stage termed monoclonal gammopathy of undetermined significance. In some patients, an intermediate asymptomatic but more advanced pre-malignant stage termed smouldering (or indolent) MM can be recognized.

MM is characterized by a high degree of variability in the disease course and a heterogeneous clinical course. Several parameters have been identified that can be used to assess risk and prognosis including serum beta2-microglobulin, albumin, C-reactive protein and lactate dehydrogenase. The International Staging System uses the combination of the serum beta2-microglobulin and albumin level and consists of 3 stages (stage III=poorest outcome). Also, genetic abnormalities, including chromosomal translocations, deletions, duplications, and genetic mutations are used for patient stratification and as as prognostic factors.

Newly diagnosed (ND) myeloma patients are treated if they have CRAB criteria i.e. hypercalcemia (calcium >11.0 mg/dL), renal failure (creatinine >2.0 mg/mL), anemia (hemoglobin <10 g/dL), or any of the three new myeloma defining events as free light chain (FLC)>100, plasma cells in the bone marrow >60%, focal lesions in the magnetic resonance imaging (MRI) ([1]).

In 2015, the SLAMF7-specific monoclonal antibody huLuc63 received FDA approval for the treatment of multiple myeloma under the trademark “Elotuzumab” and the EU-wide approval was granted in 2016. The elotuzumab antibody contains the variable heavy and light chains of muLuc63 antibody and the constant heavy and light chains of human IgG1.

The SLAMF7-specific antibody elotuzumab is indicated to be used only in combination with lenalidomide and dexamethasone for the treatment of myeloma patients. The antibody exerts its therapeutic effect by targeting SLAMF7 on myeloma cells and facilitating the interaction with natural killer cells to mediate the killing of myeloma cells through antibody-dependent cellular cytotoxicity (ADCC) [2, 3].

In a randomized Phase III study (ELOQUENT-2), the rate of progression-free survival in the elotuzumab group was 68% at one year after beginning of treatment. In the control group, where patients received only Lenalidomid and dexmethason, the rate of progression-free survival was 57% [Lonial S, N Engl J Med, 2015]. After three years, the interim overall survival rate was 60% in the elotuzumab group versus 53% in the control group [4].

Since the SLAMF7 antigen is also expressed on some subpopulations of normal lymphocytes the on-target off-tumor cytotoxic effect on autologous cells was analysed in the elotuzumab studies. In the ELOQUENT-2 study, a stronger effect in lymphocyte reduction after the initial infusion was reported in the elotuzumab group compared to the control group (77% versus 49%).

Whilst MM survival has significantly improved in the past years with the incorporation of new agents (proteasome inhibitors, immunomodulatory imide drugs [IMiDs], monoclonal antibodies [mAbs]), the majority of patients will eventually relapse and further treatments will be needed.

The clinical course of the disease is characterized by a relapse/remitting course with durations of response that shortens with each relapse leading to a refractory phase in which treatment options are few and survival times are short.

Moreover, if patients relapse after therapy with (and are refractory to) proteasome inhibitors, IMiDs, and mAbs (anti-CD38), OS is less than 9 months.

This group of patients has an unmet medical need for innovative and effective therapy.

Furthermore, adoptive immunotherapy with gene-engineered chimeric antigen receptor (CAR)-T cells is a transformative novel treatment modality in hematology and oncology. CARs are synthetic receptors with an extracellular antigen-binding domain derived from the variable heavy and light chains of a monoclonal antibody and an intracellular signaling module that mediates T cell activation after antigen-binding. Target molecules that are expressed on malignant cells but not on vital normal tissues can be targeted by CAR-T cells. Clinical data has been obtained by CAR-T cell immunotherapy with cluster of differentiation (CD) 19-specific CAR-T cells in B-cell leukemia and lymphoma. However, CAR-T cell therapy can be accompanied by severe side effects as CRS and neurotoxicity which may be the consequence of strong CAR-T activation, cytokine release and ensuing systemic inflammation.

Accordingly, there remains the need for novel therapies that provide a safe and more effective treatment.

### DESCRIPTION OF INVENTION

The present invention aims to overcome the unmet clinical needs by providing an improved composition for therapeutic treatment of patients.

The present inventors have performed extensive experimental tests in order to support the suitability of SLAMF7 CAR-T cells which are derived from the MM patient for the treatment of cancer. Specifically, the SLAMF7 CAR-T cells are obtained by gene-transfer reagents using Sleeping Beauty (SB) transposase SB100X mRNA and SLAMF7 CAR-encoding DNA minicircle. It has been confirmed based on the experimental results that SB transposition accomplishes stable gene-transfer and a favourable genomic integration profile of CAR transposons with a higher rate of integrations into genomic safe harbours compared to viral gene-transfer vectors. Thus, the safety of this gene transfer system used to generate the transformed T cell of the present invention is considered to be higher than that of viral vectors.

The inventors of the present application also demonstrated that SLAMF7 CAR T-cells prepared by Sleeping Beauty gene transfer confer superior anti-myeloma efficacy in vivo compared to SLAMF7 CAR T-cells prepared by lentiviral gene transfer. Hence, SLAMF7 CAR T-cells that are prepared by virus-free SB gene transfer possess greater anti-myeloma efficacy and therapeutic potential, which leads to significantly improved clinical activity, and significantly improved clinical outcome.

Moreover, the pharmaceutical composition of the present invention is further defined with respect to the ratio of recombinant CD4⁺ T cells to recombinant CD8⁺ T cells. Thereby, it has become possible to identify CAR-T cell doses that are safe and effective.

Accordingly, the present invention provides the following preferred embodiments:

1. A SLAMF7 binding chimeric antigen receptor (CAR) polypeptide, comprising at least one extracellular ligand binding domain, a transmembrane domain and at least one intracellular signalling domain, wherein said extracellular ligand binding domain comprises a SLAMF7-binding element and an IgG4-FC spacer domain, wherein said transmembrane domain comprises a CD28 transmembrane domain, and wherein said intracellular signalling domain comprises a costimulatory domain and a CD3 zeta domain.
2. The SLAMF7 binding CAR polypeptide according to item 1, wherein the SLAMF7-binding element is represented by an amino acid sequence shown in SEQ ID NO: 1 or by an amino acid sequence having at least 90% identity to an amino acid sequence shown in SEQ ID NO: 1.
3. The SLAMF7 binding CAR polypeptide according to items 1 or 2, wherein the IgG4-FC spacer domain is represented by an amino acid sequence shown in SEQ ID NO: 2 or by an amino acid sequence having at least 90% identity to an amino acid sequence shown in SEQ ID NO: 2.
4. The SLAMF7 binding CAR polypeptide according to any one of the proceeding items, wherein the CD28 transmembrane domain is represented by an amino acid sequence shown in SEQ ID NO: 3 or by an amino acid sequence having at least 90% identity to an amino acid sequence shown in SEQ ID NO: 3.
5. The SLAMF7 binding CAR polypeptide according to any one of the preceding items, wherein the costimulatory domain is a CD28 cytoplasmic domain or a 4-1BB costimulatory domain.
6. The SLAMF7 binding CAR polypeptide according to any one of the preceding items, wherein the costimulatory domain is a CD28 cytoplasmic domain.
7. The SLAMF7 binding CAR polypeptide according to any one of the proceeding items, wherein the CD28 cytoplasmic domain is represented by an amino acid sequence shown in SEQ ID NO: 4 or by an amino acid sequence having at least 90% identity to an amino acid sequence shown in SEQ ID NO: 4.
8. The SLAMF7 binding CAR polypeptide according to any one of the proceeding items, wherein the 4-1BB costimulatory domain is represented by an amino acid sequence shown in SEQ ID NO: 25 or by an amino acid sequence having at least 90% identity to an amino acid sequence shown in SEQ ID NO: 25.
9. The SLAMF7 binding CAR polypeptide according to any one of the proceeding items, wherein the CD3 zeta domain is represented by an amino acid sequence shown in SEQ ID NO: 5 or by an amino acid sequence having at least 90% identity to an amino acid sequence shown in SEQ ID NO: 5.
10. The SLAMF7 binding CAR polypeptide according to any one of items 1-7 and 9, wherein said extracellular domain comprises an amino acid sequence shown in SEQ ID NO: 6 or an amino acid sequence having at least 90% identity to an amino acid sequence shown in SEQ ID NO: 6, said transmembrane domain comprises an amino acid sequence shown in SEQ ID NO: 3 or an amino acid sequence having at least 90% identity to an amino acid sequence shown in SEQ ID NO: 3 and said intracellular signalling domain comprises an amino acid sequence shown in SEQ ID NO: 7 or an amino acid sequence having at least 90% identity to an amino acid sequence shown in SEQ ID NO: 7.
11. The SLAMF7 binding CAR polypeptide according to item 10, wherein the CAR polypeptide comprises an amino acid sequence shown in SEQ ID NO: 8 or an amino acid sequence having at least 90% identity to an amino acid sequence shown in SEQ ID NO: 8.
12. A polynucleotide encoding the SLAMF7-CAR polypeptide according to any one of the preceding items.
13. The polynucleotide according to item 12, wherein the polynucleotide further comprises flanking segments in 5′-direction and in 3′-direction of the polynucleotide encoding the SLAMF7-CAR polypeptide.
14. The polynucleotide according to item 13, wherein the flanking segment in 5′-direction is a left inverted repeat/direct repeat (IR/DR) segment and the flanking segment in 3′-direction is a right inverted repeat/direct repeat (IR/DR) segment.
15. The polynucleotide according to item 14, wherein the left IR/DR segment is represented by SEQ ID NO: 9 and right IR/DR segment is represented by SEQ ID NO: 10.
16. The polynucleotide according to any one of items 12 to 15, wherein the polynucleotide comprises a nucleotide sequence of a left IR/DR, a polynucleotide sequence encoding the SLAMF7-CAR polypeptide and a nucleotide sequence of a right IR/DR.
17. The polynucleotide according to any one of items 12 to 16, wherein the polynucleotide comprises a nucleotide sequence represented by SEQ ID NO: 11.
18. An expression vector comprising a polynucleotide according to any one of item 12-17.
19. The expression vector according to item 18, wherein the expression vector is a minimal DNA expression cassette.
20. The expression vector according to items 18 or 19, wherein expression vector is a transposon donor DNA molecule.
21. The expression vector according to any one of items 18 to 20, wherein the expression vector is a minicircle DNA.
22. The expression vector according to any one of items 18 to 21, comprising a polynucleotide sequence shown in SEQ ID NO: 11.
23. The expression vector according to any one of items 18 to 22, comprising a polynucleotide sequence shown in SEQ ID NO: 12.
24. A recombinant immune cell comprising a polynucleotide according to any one of items 12-17.
25. The recombinant immune cell according to item 24, wherein the polynucleotide is located in the nuclear genome of the immune cell.
26. The recombinant immune cell according to items 24 or 25, wherein the polynucleotide is expressed.
27. The recombinant immune cell according to any one of the items 24 to 26, wherein said recombinant immune cell is a recombinant lymphocyte.
28. The recombinant immune cell according to item 27, wherein said recombinant lymphocyte is a recombinant T cell.
29. The recombinant immune cell according to item 28, wherein said recombinant T cell is a recombinant CD4⁺ cell or a recombinant CD8⁺ cell.
30. The recombinant immune cell according to any one of the items 24 to 29, further expressing EGFRt.
31. The recombinant immune cell according to any one of the items 24 to 30, wherein said recombinant immune cell is a recombinant human cell.
32. The recombinant immune cell according to any one of the items 24 to 31, wherein said recombinant immune cell does not comprise an amino acid sequence of the SB transposase as represented by SEQ ID NO: 13 or fragments thereof in a detectable amount at day 14 after gene transfer.
33. Method for producing recombinant immune cells, comprising the steps of
(a) isolating immune cells from a blood sample of a subject
(b) transforming immune cells using a transposable element comprising a polynucleotide according to any one of items 12 to 17 and a Sleeping Beauty (SB) transposase to produce recombinant immune cells
(c) purifying immune cells.
   34. The method according to item 33, wherein the immune cell is a lymphocyte.
   35. The method according to item 34, wherein the lymphocyte is a T cell.
   36. The method according to item 35, wherein the T cell is a CD4⁺ cell and/or a CD8⁺ cell.
   37. The method according to item 36, wherein the recombinant CD4⁺ T cells and the recombinant CD8⁺ T cells are expanded separately.
   38. The method according to any one of items 33-37, wherein the subject is a human.
   39. The method according to any one of items 33-38, wherein a plurality of recombinant CD4⁺ T cells and a plurality of recombinant CD8⁺ T cells are combined in a defined ratio to form a composition of recombinant T cells, wherein the ratio of said recombinant T cells in the composition is in the range of 0.5:1 to 2:1.
   40. The method according to item 39, wherein the ratio of the recombinant CD4+ T cells and recombinant CD8+ T cells in the composition is 1:1.
   41. The method according to any one of items 33-40, wherein the SB transposase is represented by an amino acid sequence shown in SEQ ID NO: 13.
   42. The method according to any one of items 33-41, wherein the recombinant immune cells do not comprise an amino acid sequence of the SB transposase as represented by SEQ ID NO: 13 or fragments thereof in a detectable amount at day 14 after gene transfer.
   43. A recombinant immune cell obtainable by the method of any one of items 33-42.
   44. A pharmaceutical composition comprising a plurality of recombinant immune cells according to any one of items 24 to 32 or of item 43.
   45. A pharmaceutical composition according to item 44 for use as a medicament.
   46. A pharmaceutical composition according to any one of items 44 or 45 for use in a method of treating cancer, wherein in the method the pharmaceutical composition is to be administered to a subject.
   47. The pharmaceutical composition for use according to any one of items 45 or 46, wherein the pharmaceutical composition to be administered comprises recombinant immune cells in a dose of about 1×10⁴ cells/kg body weight, of about 3×10⁴ cells/kg body weight, of about 1×10⁵ cells/kg body weight, of about 3×10⁵ cells/kg body weight, of about 1×10⁶ cells/kg body weight, of about 3×10⁶ cells/kg body weight, of about 1×10⁷ cells/kg body weight, of about 3×10⁷ cells/kg body weight, of about 1×10⁸ cells/kg body weight, of about 3×10⁸ cells/kg body weight, of about 1×10⁹ cells/kg body weight, or of about 3×10⁹ cells/kg body weight.
   48. The pharmaceutical composition for use according to any one of items 45 to 47, wherein the pharmaceutical composition is to be administered intravenously.
   49. The pharmaceutical composition for use according to any one of items 45 to 48, wherein the recombinant immune cells are to be administered in a single dose.
   50. The pharmaceutical composition for use according to any one of items 45 to 48, wherein the recombinant immune cells are to be administered in multiple doses.
   51. The pharmaceutical composition for use according to any one of items 45 to 50, wherein said recombinant immune cells are recombinant lymphocytes.
   52. The pharmaceutical composition for use according to item 51, wherein said recombinant lymphocytes are recombinant T cells.
   53. The pharmaceutical composition for use according to item 52, wherein said recombinant T cells are CD4⁺ T cells and/or CD8⁺ T cells.
   54. The pharmaceutical composition for use according to item 53, wherein said recombinant T cells are present in a defined ratio.
   55. The pharmaceutical composition for use according to item 54, wherein said ratio is in a range of 0.5:1 to 2:1.
   56. The pharmaceutical composition for use according to item 55, wherein said ratio is about 1:1.
   57. The pharmaceutical composition for use according to any one of items 45 to 56, wherein said subject is a human.
   58. The pharmaceutical composition for use according to any one of item 45 to 57, wherein said cancer is multiple myeloma.

### BRIEF DESCRIPTION OF THE DRAWINGS

FIG. 1: Structure of the gene cassette

A) Structure of the gene cassette comprising the SLAMF7 CAR and the EGFRt sequence separated by a T2A ribosomal skip element. B) After gene modification, the EGFRt protein and the SLAMF7 CAR are both expressed on the cell surface. The annotated transgene sequence of the SLAMF7 CAR construct, with a clear delineation which parts of the protein sequence belong to which element is described.

FIG. 2: Denaturing agarose gel electrophoresis of SB100X mRNA

The manufactured SB mRNA is of high purity with an expected length of approximately 1300 nt. Shown is a single band of SB100X mRNA (in lane 2) running between the 1000 nt and 1500 nt marker bands (FlashGel RNA Marker Lonza, lane 1), which is in agreement with the expected length of app. 1300 nt.

FIG. 3: Result of the microfluidic capillary electrophoresis (Experion System, Bio-Rad): Electropherogram of SB100X mRNA

The manufactured SB mRNA is of high purity with an expected length of approximately 1300 nt. Shown is a single band of SB100X mRNA (in lane 2) running between the 1000 nt and 1500 nt marker bands (FlashGel RNA Marker Lonza, lane 1), which is in agreement with the expected length of app. 1300 nt.

FIG. 4: Steps of SLAMF7 CAR-T cell manufacturing process

FIG. 5: EGFRt expression on T cells of the pharmaceutical composition

DP cells were stained for CD4, CD8 and EGFRt expression. Left dot plot shows flowcytometric data of CD4⁺ T cells, right dot plot of CD8⁺ T cells. EGFRt=truncated epidermal growth factor receptor.

FIG. 6: T cell subsets in the DP

Cells of the formulated DP were stained for the expression of the T cell differentiation markers. Cells were first gated on CD4 (upper plots) and CD8 expression (lower plots), and then on the expression of the differentiation markers CD62L, CD45RA and CD45RO.

FIG. 7: Activation/exhaustion markers on the DP

Cells of the formulated DP were stained for the expression of the T cell exhaustion markers. Cells were first gated on the expression of CD4 (upper plots) and CD8 expression (lower plots) and afterwards on the expression of exhaustion markers PD-1, LAG-3 and TIM-3.

FIG. 8: Vector copy numbers in DP SLAMF7 CAR-T cells

The vector copy numbers in DP cells from validation runs was determined by quantitative droplet digital PCR. PCR=polymerase chain reaction.

FIG. 9: Canonical SB transposon integrations in the genomes of DP cells

Nucleotide frequencies of the majority rule consensus sequences of all insertion sites obtained with the three SLAMF7 CAR validation experiments, each (n=3 samples corresponding to n=3 validation runs). Insertion site logos were calculated and plotted with the SeqLogo package (PMID: 2172928). The x-axis shows the majority rule nucleotide sequence within the 60-nucleotide-long windows centered on the insertion sites. The ‘Information Content’ depicted on the y-axis stands for the frequency of the nucleotides at each position with the maximum value of 2 (log 2 4). Thus, the consensus logo depicts the degree of conservation of each position using the height of the consensus character at that position. The Sleeping Beauty transposons are known to integrate almost exclusively into a TA target di-nucleotides (PMID: 9390559) which are in the center of the ATATATAT consensus motif (PMID: 12381300). Our analyses of the insertion sites of all three validation runs showed the expected insertion sites pattern what has been found for SB transposons mobilized from conventional donor plasmids and minicircles.

FIG. 10: Genome-wide distribution of SLAMF7 CAR SB transposons in the DP T cell genome

Distribution of SB insertions in functional genomic segments of human T cells. Numbers show relative enrichment above the random frequency (set to 1). Colour intensities depict the degree of deviation from the expected random distribution (red: overrepresentation; blue: underrepresentation). downTES10 kb stands for genomic regions extending 10 kb downstream from the transcriptional end sites of genes. upTSS10 kb indicate 10-kb-long genomic segments upstream of transcriptional start sites of genes.

FIG. 11: Absence of detectable residual SB100X transposase in DP cells

A volume of cell extract corresponding to 1×10⁶ cells of each validation run was subjected to SDS-PAGE alongside recombinant SB100X protein in concentrations ranging from 0 pg −1 ng and blotted onto a nitrocellulose membrane for subsequent chemiluminescent Western blotting. Exposure with a-Histone H3 antibody (loading control) was 30 sec, with a-SB antibody 20 min.

FIG. 12: Residual SB100X transposase in SB-RP cells one day and 12 days after transfection

A volume of SB-RP cell extract corresponding to 1×10⁶ cells was subjected to SDS-PAGE alongside 1 ng of recombinant SB100X protein. It was blotted onto a nitrocellulose membrane for subsequent Western blotting. Untransfected T cells were used as negative control. T cells extracts were gained on day 3 (one day after nucleofection) and on day 14 (12 days after transfection, day of harvesting) of the manufacturing process. Exposure with a-Histone H3 antibody (loading control) was 7 sec, with a-SB antibody 5 min.

FIG. 13: EGFRt and CAR expression on SLAMF7 CAR-T cells

CD4⁺ T cells were transfected with the SLAMF7 CAR-EGFRt gene cassette or left unmodified as control. T cells were single or double-stained for CAR expression with human SLAMF7 protein linked to a Twin-Strep Tag and ImmoChromeo488 fluorescent anti-Strep Tag antibody and for EGFRt expression with APC-labeled anti-EGFRt antibody. EGFRt=truncated epidermal growth factor receptor.

FIG. 14: Specific cytotoxicity of DP cells measured by europium release assay

Cytotoxic capacity of SLAMF7 CAR-T cells towards SLAMF7-positive target cells (K562 SLAMF7, MM.1S) or SLAMF7-negative control cells (K562) was measured by europium release assay after 2 hours of coincubation. E:T=effector:target cell ratio, n=1 donor, data collected as technical triplicates.

FIG. 15: Specific cytotoxicity of DP cells measured by bioluminescence-based assay

Cytotoxic capacity of SLAMF7 CAR-T cells towards SLAMF7-positive target cells (OPM-2, MM.1S, K562 SLAMF7) or SLAMF7-negative control cells (K562) was measured by bioluminescence-based assay after 4 and 24 hours of coincubation. DP=drug product, E:T=effector:target cell ratio, n=1 donor, data collected as technical triplicates.

FIG. 16: Specific lysis by CD8⁺ SB-RP cells

Cytotoxic capacity of CD8⁺ SLAMF7 CAR-T cells was tested in a 4-hour and 24-hour bioluminescence-based cytotoxic assay. SLAMF7-positive cells (K562 SLAMF7, MM.1S, OPM-2) or SLAMF7-negative cells (K562) were used as targets. E:T=effector:target ratio, mean values+/−SEM of n=3 or n=4 donors.

FIG. 17: Specific cytotoxicity/cytolysis of CD4⁺ and CD8⁺ LV-RP

Cytotoxic capacity of CD4⁺ and CD8⁺ LV-RP was tested in a 4-hour and 20-hour cytotoxic assay. SLAMF7-positive cells (K562 SLAMF7, MM.1S, OPM-2) or SLAMF7-negative cells (K562) were used as targets E:T=effector:target ratio. Representative data of the results obtained in independent experiments with CAR-T cells prepared from n=4 healthy donors. data collected as technical triplicates ([5]).

FIG. 18: Cytokine release of DP cells

Cytokine release upon 20 hours co-culture of DP cells or unmodified T cells with SLAMF7-positive target cells (K562 SLAMF7, MM.1S, OPM-2, NCI-H929) or control cells (K562) was measured by Interleukin-2 and Interferon-y ELISA. Medium only served as negative control, medium with PMA/lonomycin as positive control. DP=drug product, ELISA=enzyme-linked immunosorbent assay, n=1 donor, data collected as technical triplicates.

FIG. 19: Cytokine release of CD4⁺ and CD8⁺ SB-RP cells

Cytokine release upon 20 hours co-culture of SLAMF7 CAR-T cells or unmodified T cells with SLAMF7-positive target cells (K562 SLAMF7, MM.1S, OPM-2) or control cells (K562) was measured by Interleukin-2 and Interferon-y ELISA. CD4⁺ and CD8⁺ T cells were tested separately. mean values+/−SEM of n=4 donors.

FIG. 20: Cytokine release of CD4⁺ and CD8⁺ LV-RP cells

Cytokine release upon 20 hours co-culture of SLAMF7 CAR-T cells or unmodified T cells with SLAMF7-positive target cells (K562 SLAMF7, MM.1S, OPM-2, NCI-H929) or control cells (K562) was measured by Interleukin-2 and Interferon-y ELISA. CD4⁺ and CD8⁺ T cells were tested separately. Representative data of the results obtained in independent experiments with CAR-T cells prepared from 5 healthy donors. Data was collected as technical triplicates.

FIG. 21: Antigen-specific proliferation of CD4⁺ and CD8⁺ DP cells

Proliferation upon 72 hours co-culture of SLAMF7 CAR-T cells (red) or unmodified T cells (blue) with SLAMF7-positive target cells (K562 SLAMF7, MM.1S, OPM-2, NCI-H929) or control cells (K562) was measured by CFSE dilution. As negative control, cells were left untreated (Medium), as positive control they were stimulated with Interleukin-2. The formulated DP contained a mixture of CD4⁺ and CD8⁺ T cells. CFSE=carboxyfluorescein diacetate succinimidyl ester, n=1.

FIG. 22: Antigen-specific proliferation of CD4⁺ and CD8⁺ SB-RP cells

Proliferation upon 72 hours co-culture of SLAMF7 CAR-T cells (red) or unmodified T cells (blue) with SLAMF7-positive target cells (K562 SLAMF7, MM.1S, OPM-2, NCI-H929) or control cells (K562) was measured by CFSE dilution. CD4⁺ and CD8⁺ T cells were stimulated separated from each other. CFSE=carboxyfluorescein diacetate succinimidyl ester, representative data of the results obtained in independent experiments with CAR-T cells prepared from n=2 healthy donors.

FIG. 23: Antigen-specific proliferation of CD4⁺ and CD8⁺ LV-RP cells

Proliferation after 72 hours co-culture of LV-RP cells (red) or unmodified T cells (blue) with SLAMF7-positive target cells (K562 SLAMF7, MM.1S, OPM-2, NCI-H929) or control cells (K562) was measured by CFSE dilution. CD4⁺ and CD8⁺ T cells were stimulated separated from each. Representative data of the results obtained in independent experiments with CAR-T cells prepared from n=4 healthy donors ([5]).

FIG. 24: Anti-myeloma efficacy of DP in vivo (CARAMBA_Val #1)

NSG mice were inoculated with MM.1S tumor cells and after 8 days treated with 5×10⁶/2.5×10⁶ SLAMF7 CAR-T cells, unmodified T cells of the same donor, or were left untreated. 24-1) Bioluminescence imaging of the mice shows tumor cell distribution at different time points. 24-2) Average radiance measured by bioluminescence imaging in each single mouse at different time points. 24-3) Kaplan-Meyer-survival curve of mouse groups (d=day).

FIG. 25: Anti-myeloma efficacy of DP in vivo (CARAMBA_Val #3)

NSG mice were inoculated with MM.1S tumor cells and after 14 days treated with 5 Mio SLAMF7 CAR-T cells, 6.9 Mio unmodified T cells of the same donor, or were left untreated. 25-1) Bioluminescence imaging of mice at different time points. 25-2) Average radiance measured by bioluminescence imaging in each single mouse at different time points. 25-3) Kaplan-Meyer-survival curve of mouse groups (d=day).

FIG. 26: Anti-myeloma efficacy of LV-RP cells in vivo

NSG mice were i.v. inoculated with ffluc_eGFP-transduced MM.1S myeloma cells and, 14 days later, treated with a single dose of SLAMF7 CAR-T cells or CD19 CAR control T cells (both i.v., 2.5×10⁶ CD4⁺ and 2.5×10⁶ CD8⁺ CAR-T cells) or remained untreated (n=5 per group). A) Serial bioluminescence imaging to assess myeloma progression/regression. B) Flow cytometric analysis of peripheral blood (PB), bone marrow (BM) and spleens (SP) to detect residual MM.1S myeloma cells in exemplary mice that were euthanized on day 35 after tumor inoculation. C) Waterfall plot shows the relative increase/decrease in bioluminescence signal between day 14 (before treatment) and day 20 (6 days after treatment) in individual mice. A-C) The data shown are representative of 3 independent experiments prepared from 3 healthy donors ([5]).

FIG. 27: Anti-myeloma efficacy of patient-derived LV-RP in vivo

A) Serial bioluminescence imaging of NSG/MM.1S mice that were treated with SLAMF7 CAR or CD19 control CAR-T cells or remained without treatment. T cells were administered on day 14 after tumor inoculation B) Average radiance evaluated by serial bioluminescence imaging in each treatment group (n=4 per group, ** p<0.01) C) Waterfall plot shows the relative increase/decrease of bioluminescence signal between day 14 (before treatment) and day 24 (10 days after treatment) in each mouse. D) Kaplan-Meier analysis of survival ([5]).

FIG. 28: Recognition of primary myeloma cells by autologous LV-RP cells

Primary myeloma cells were labeled with eFluor670 fluorescent dye and cocultured with autologous SLAMF7-CAR or CD19-CAR (control) CD8⁺ T cells (10 000 myeloma target cells, E:T ratio 10:1 to 1:1). After 4 hours of incubation, live (7-AAD⁻) CD138⁺ eFluor⁺ myeloma cells were quantified by flow cytometry using counting beads and specific lysis calculated using untreated myeloma cells as a comparator ([5]).

FIG. 29: Recognition of primary myeloma cells by autologous LV-RP cells from newly diagnosed or relapsed/refractory MM patients

A) Primary myeloma cells were labeled with eFLuor670 fluorescent dye and cocultured with autologous SLAMF7 CAR or CD19 CAR CD8⁺ T cells at different E:T ratios. After 4 hours of incubation, live myeloma cells were quantified by flow cytometry using counting beads and specific lysis calculated using untreated myeloma cells as a comparator. Specific lysis of primary myeloma cells obtained from a patient with newly diagnosed (ND, left) and a patient with relapsed/refractory multiple myeloma (R/R, right) by autologous SLAMF7 CAR or CD19 CAR (control) T cells (E:T ratio 10:1). Native K562 cells and K562 SLAMF7 cells were included as a negative and positive control, respectively ([5]).

FIG. 30: Phenotype of CD8⁺ SB-RP and LV-RP cells from the same donor

CD8⁺ SLAMF7 CAR-T cells of the same donor were either produced by lentiviral gene transfer or SB transposition. The further manufacturing steps were equal. The cells were sorted for EGFRt expression and expanded with feeder cells. The SLAMF7 CAR-T cells or unmodified control T cells were stained for CD8, EGFRt and SLAMF7 expression and analysed by flow cytometry.

FIG. 31: Specific cytotoxicity of CD8⁺ SB-RP/LV-RP cells from the same donor

CD8⁺ T cells from the same donor were gene modified by lentivirus or SB transposition. The further manufacturing steps were equal. The cells were sorted for EGFRt expression and expanded with feeder cells. The SLAMF7 CAR-T cells were functionally tested in a 4-hour and 24-hour cytotoxic assay using SLAMF7-positive (OPM-2, MM.1S, K562 SLAMF7) and SLAMF7-negative (K562) target cells. E:T=effector:target ratio.

FIG. 32: Cytokine release of CD4⁺ and CD8⁺ CAR T cells derived from the same donor and generated by SB transposition or LV transduction

T cells from one donor were gene modified by lentivirus or SB transposition to express the SLAMF7 CAR. After gene-transfer, all manufacturing steps were identical: after expansion, T cells were sorted for EGFRt-expression and expanded with irradiated, antigen-presenting feeder cells. For analyzing cytokine release, SLAMF7 CAR-T cells were co-cultured for 20 hours with SLAMF7-positive (OPM 2, MM.1S, K562 SLAMF7) and SLAMF7-negative (K562) target cells. Medium only served as negative control, medium with PMA/lonomycin as positive control. Cytokines in the supernatant were analyzed by Interleukin-2 and Interferon-y ELISA assay. ELISA=enzyme-linked immunosorbent assay, n=1 donor, data collected as technical triplicates.

FIG. 33: In vitro effect of ‘conventional’ anti-MM drugs compared to LV-RP cells on MM.1S cells

A) Cytolytic activity of CD8⁺ LV-RP cells against MM.1S evaluated in a 20-hour cytotoxicity assay at distinct effector to target cell ratios (E:T 20:1, 10:1, 5:1, CAR-T cells lentivirally produced). B) Elotuzumab (huLuc63, SLAMF7 mAb) triggers SLAMF7-specific cell lysis in a dose-dependent manner. ADCC was performed by incubating calcein-AM-labeled target MM.1S cells with human PBMC effector cells at an E:T ratio of 10:1, in the presence of various concentrations of huLuc63 (solid squares) or iso IgG1 (open squares) ([3]). C) Efficacy of the in vitro combinations of Panobinostat with other anti-myeloma agents in MM. MTT studies of the double and triple combinations of Panobinostat (1 nM) with Dexamethasone (5 nM) and Lenalidomide (0.5 μM) or Bortezomib (2 nM) in the cell line MM.1S after 72 hours of treatment. ([6]). D) Synergistic anti-myeloma activity of Panobinostat (LBH589) in combination with Melphalan. MM.1S cells were treated with a constant concentration of LBH589 and increasing concentrations of Melphalan for 48 hours. Cell viability was measured with the MTS assay ([7]). ADCC=antibody-dependent cell-mediated cytotoxicity, E:T=effector:target ratio, MTT=3-(4,5-di methylthiazol-2-yl)-2,5-dephenyltetrazolium bromide, MTS=(3-(4,5-dimethylthiazol-2-yl)-5-(3-carboxymethoxyphenyl)-2-(4-sulfophenyl)-2H-tetrazolium).

FIG. 34: In vitro effect of ‘conventional’ anti-MM drugs compared to LV-RT cells on primary MM cells

A) Specific lysis of primary MM cells by LV-RP cells. B) SLAMF7⁺ CD138⁺ MM cells from 2 patients were cultured in the presence of titrated huLuc63 mAb (=Elotuzumab, SLAMF7 mAb). Cell viability was determined by MTT assay. ([3]) C) Primary CD38⁺ CD138⁺ cells were incubated with 100 μg/mL SAR650984 (=Isatuximab, CD38 mAb) for 18 hours. ([8]) D) Bone marrow samples from 2 patients with MM and a patient with PCL were treated ex vivo for 24 h with Panobinostat (20 nM), Dexamethasone (40 nM) and Bortezomib (5 nM). Samples were incubated with Annexin V and CD38, CD45, CD56 monoclonal antibodies to analyze the induction of apoptosis in the clonal population of plasma cells ([6]). MM=multiple myeloma, PCL=plasma cell leukemia, MTT=3-(4,5-dimethylthiazol-2-yl)-2,5-dephenyltetrazolium bromide.

FIG. 35: DP cells in the peripheral blood of mice during tumor relapse

Flow cytometric analysis of peripheral blood from mouse 81-1 and mouse 81-3 treated with DP cells during tumor relapse at day 76/day 70 after MM.1S tumor cell inoculation. Single cells were first gated on 7-AAD negativity (living cells) and ffluc negativity (no MM.1S cells) and then on the expression of human CD45 (human lymphocytes). These cells were analyzed for the expression of CD4, CD8, EGFRt and SLAMF7 (d=day).

FIG. 36: Comparison of anti-myeloma efficacy of SLAMF7 CAR T-cells that had been prepared by Sleeping Beauty gene transfer (SB) vs. lentiviral gene transfer (LV) in a murine xenograft model (NSG/MM1.S)

36-1: Kaplan-Meier analysis of survival shows anti-myeloma efficacy of lentivirally generated SLAMF7 CAR T-cells in vivo. 36-2: Kaplan-Meier analysis of survival shows anti-myeloma efficacy of SLAMF7 CAR T-cells generated by Sleeping Beauty gene transfer. 36-3: T cell kinetic in mice during tumor regression and relapse. NSG mice were inoculated with 2×10⁶ MM.1S/ffluc cells. After 14 days they were treated with a single dose of 5×10⁶ SLAMF7 CAR T cells generated by Sleeping beauty gene transfer. CAR-T cell persistence was measured in peripheral blood.

FIG. 37: Binding capacity of the SLAMF7 CAR to non-human SLAMF7 proteins

The binding capacity of LV-RP cells against SLAMF7 molecules of different species was analyzed by flow cytometry (lower row). SLAMF7 molecules linked to a Twin-Strep Tag were stained by an anti-Strep Tag antibody. CD19 CAR-T cells were used as controls (upper row).

FIG. 38: Reactivity of LV-RP cells to non-human SLAMF7 proteins

CD4⁺ LV-RP cells were incubated on 96-well plates coated with SLAMF7 molecules of different species (blue bars). Cytokine release was measured by enzyme-linked immunosorbent assay of supernatants. CD4⁺ CD19 CAR-T cells were used as control to measure background cytokine release (red bars). The bars marked with # are cut off, as they dramatically exceed the top standard value of 500 pg/ml IL-2.

FIG. 39: SLAMF7 expression on patient lymphocytes

Expression of SLAMF7 on normal lymphocyte subsets obtained from peripheral blood of myeloma patients (n=10) analyzed by flow cytometry using an anti-SLAMF7 antibody. The diagram shows the mean percentage of SLAMF7^{-/high} CD8 T cells (CD3⁺, CD4⁻, CD8⁺), CD4 T cells (CD3⁺, CD4⁺, CD8⁻), γδ T cells (Vγ9δ2 TCR⁺), NKT cells (CD3⁺, CD56⁺), NK cells (CD3⁻, CD56⁺), B cells (CD3⁻, CD19⁺) and monocytes (CD3⁻, CD14⁺; [5]).

FIG. 40: Selective killing of SLAMF7^{+/high} CD8⁺ T cells by DP cells

eFluor-labeled CD8⁺ T cells were cultured with autologous DP cells or control cells at a 4:1 effector to target cell ratio for 24 hours. The diagram shows the mean percentage of residual live (7-AAD-negative) target cells (left) and their SLAMF7 expression (right). data collected as technical triplicates.

FIG. 41: Selective killing of SLAMF7^{+/high} normal lymphocytes by SB-RP cells

PBMC were cultured with autologous CD8⁺ SB-RP cells for 12 hours at a 4:1 effector to target cell ratio. The subset composition, viability and SLAMF7-expression of PBMCs was determined by flow cytometry by staining for CD8 T cells (CD3⁺, CD4⁻, CD8⁺), CD4 T cells (CD3⁺, CD4⁺, CD8⁻), NK cells (CD3⁻, CD56⁺) and B cells (CD3⁻, CD19⁺), as well as for 7-AAD and SLAMF7.

FIG. 42: Selective killing of SLAMF7^{+/high} normal lymphocytes by LV-RP cells

A) The diagram shows the mean percentage of residual live (7-AAD-negative) cells in each of the normal lymphocyte subsets after co-culture with SLAMF7-CAR (lentivirus-based) or control CD19 CAR-T cells. Data shown are representative for 4 independent experiments. B) CD8⁺ T cells were isolated from peripheral blood of myeloma patients, labelled with eFluor670, and used as target cells in 12-hour coculture assays with autologous CD8⁺ SLAMF7 CAR (lentivirus-based) and control CD19 CAR-T cells (non-eFluor labelled, E:T ratio=4:1). The percentage of viable eFluor670⁺ target cells before and after co-culture was determined by staining with viability dye (top row of histograms); expression of SLAMF7 on viable target cells before and after co-culture was determined by staining with SLAMF7 antibody (middle row) and the ability of viable target cells to produce IFNγ in response to PMA (phorbol 12-myristate 13-acetate) and ionomycine stimulation before and after co-culture with CAR-T cells was determined by intracellular cytokine staining (bottom row). The dot plots show overlays of eFluor⁺ target (black) and eFluor− effector (gray) cells. The numbers in the upper quadrants provide percentages of eFluor⁺ cells ([5]).

FIG. 43: Functionality of spared CMV-specific CD8⁺ T cell fraction after fratricide

CMV-specific CD8⁺ T cell lines (CMV-CTL) were prepared from CMV-specific memory T cells and the expression of SLAMF7 was analyzed (2 top left dot plots). CMV-CTL was labelled with eFluor670 and co-cultured for 4 hours with autologous LV-RP cells or control CD19 CAR-T cells. The expression of SLAMF7 on residual living (7-AAD-negative) CMV-CTL was re-analyzed at the end of the coculture (top right dot plot). Residual living CMV-CTL was then stimulated with pp65NLV peptide-loaded K562/HLA-A2 cells, and IFNγ production in the SLAMF7^{-/high} and SLAMF7^{-/low} CMV-CTL fraction was analyzed by intracellular cytokine staining (2 bottom right dot plots). IFNγ production in SLAMF7^{-/high} and SLAMF7^{-/low} CMV-CTL before the fratricide assay was analyzed for comparison (2 bottom left dot plots) ([5]).

FIG. 44: SLAMF7 expression on SLAMF7 CAR-T cells

SB-RP cells and control T cells were stained at the end of production process with anti-SLAMF7, anti-CD4 and anti-CD8 antibodies and analyzed by flow cytometry. The percentage of SLAMF7-positive cells is depicted in the plots.

FIG. 45: ADCC of EGFRt-positive T cells by SLAMF7-negative PBMC

PBMC were sorted for SLAMF7-negativity (right) or were left unsorted (left). Afterwards, PBMC were coincubated for 24 hours with allogenic EGFRt-expressing (black bars) or unmodified (grey bars) T cells with and without addition of 50 μg/ml Cetuximab. The target T cells were previously stained with eFluor 670. The number of remaining target cells was measured by flow cytometry. Data was collected as technical triplicates.

FIG. 46: Cytotoxicity of SLAMF7 CAR-T cell batch CARAMBA_Val #1 after 24 h and 48 h storage at 2-8° C.

After 24 h or 48 h of storage SLAMF7 CAR-T cells were co-incubated for 2 hours with SLAMF7-positive target cells (K562 SLAMF7, MM.1S) or SLAMF7-negative target cells (K562). Target cell killing was measured by Europium release assay.

FIG. 47: Cytotoxicity upon 24 h co-culture of SLAMF7 CAR-T cells after 1 to 3 days of storage at 2-8° C.

After one, two and three days of storage SLAMF7 CAR-T cells were co-incubated for 24-27 hours with SLAMF7⁺ target cells (K562 SF7, OPM-2, MM.1S) or SLAMF7⁻ target cells (K562 CD19). Target cell killing was measured by bioluminescence assay.

FIG. 48: SLAMF7 CARP CD8⁺ T cells were detectable in blood derived from a patient two weeks after drug product infusion

Peripheral blood of patient D was drawn 14 days after SLAMF7 CAR-T cell infusion and analysed by flow cytometry. 3.13% of CD45⁺, CD3⁺, CD8⁺ T cells were positively stained for the SLAMF7 CAR marker EGFRt.

FIG. 49: Kinetic of SLAMF7 CAR⁺ CD8⁺ T cells in peripheral blood, body temperature and Interleukin-6 serum levels of patient D

SLAMF7 CAR⁺ CD8⁺ T cells expanded in vivo after infusion and were detectable in peripheral blood of patient D. Concurrently, body temperature and Interleukin-6 serum levels increased.

### DETAILED DESCRIPTION OF INVENTION

Unless specifically defined herein, all technical and scientific terms used herein have the same meaning as commonly understood by a skilled artisan in the fields of gene therapy, immunology, biochemistry, genetics, and molecular biology.

All methods and materials similar or equivalent to those described herein can be used in the practice or testing of the present invention, with suitable methods and materials being described herein.

The term “about” used in the context of the present invention means that the value following the term “about” may vary within the range of +/−20%, preferably in the range of +/−15%, more preferably in the range of +/−10%.

All publications, patents and patent applications cited herein are hereby incorporated by reference in their entirety for all purposes. References referred to herein are indicated by a reference number in square brackets (e.g. as “[31]” or as “reference [31]”), which refers to the respective reference in the list of references at the end of the description. In case of conflict, the present specification, including definitions, will prevail over the cited references. Further, the materials, methods, and examples are illustrative only and are not intended to be limiting, unless otherwise specified.

As used herein, each occurrence of terms such as “comprising” or “comprises” may optionally be substituted with “consisting of” or “consists of”.

### SLAMF7 Binding Chimeric Antigen Receptor (CAR) Polypeptide

The present invention relates to a SLAMF7 binding chimeric antigen receptor (CAR) polypeptide comprising at least one extracellular ligand binding domain, a transmembrane domain and at least one intracellular signalling domain, wherein said extracellular ligand binding domain comprises a SLAMF7-binding element, and an IgG4-FC spacer domain, wherein said transmembrane domain comprises a CD28 transmembrane domain, and wherein said intracellular signalling domain comprises a costimulatory domain and a CD3 zeta domain.

In an embodiment of the invention, the SLAMF7-binding element is represented by an amino acid sequence having at least 90%, preferably 95%, more preferably 97% or most preferably 99% sequence identity with an amino acid sequence shown in SEQ ID NO: 1 and has SLAMF7-binding ability. Preferably, the SLAMF7-binding element is represented by an amino acid sequence shown in SEQ ID NO: 1.

In an embodiment of the invention, the IgG4-FC spacer domain is represented by an amino acid sequence having at least 90%, preferably 95%, more preferably 97% or most preferably 99% sequence identity with an amino acid sequence shown in SEQ ID NO: 2. Preferably, the IgG4-FC spacer domain is represented by an amino acid sequence shown in SEQ ID NO: 2. The spacer connects the extracellular targeting and the transmembrane domain. It affects the flexibility of the SLAMF7-binding element, reduces the spatial constraints from CAR to antigen and therefore impacts epitope binding. Binding to epitopes with a membrane-distal position often require CARs with shorter spacer domains, binding to epitopes which lie proximal to the cell surface often require CARs with long spacer.

In an embodiment of the invention, the CD28 transmembrane domain is represented by an amino acid sequence having at least 90%, preferably 95%, more preferably 97% or most preferably 99% sequence identity with an amino acid sequence shown in SEQ ID NO: 3. Preferably, the CD28 transmembrane domain is represented by an amino acid sequence shown in SEQ ID NO: 3. The CD28 transmembrane domain consists of a hydrophobic alpha helix, traverses the membrane of the cell and anchors the CAR to the cell surface. It impacts the expression of the CAR on the cell surface.

In an embodiment of the invention, the costimulatory domain of the SLAMF7-CAR polypeptide is a CD28 cytoplasmic domain or a 4-1BB costimulatory domain.

In an embodiment of the invention, the intracellular signalling domain comprises a CD28 cytoplasmic domain and a CD3 zeta domain. In another embodiment of the invention, the intracellular signalling domain comprises a 4-1BB costimulatory domain and a CD3 zeta domain.

In an embodiment of the invention, the CD28 cytoplasmic domain is represented by an amino acid sequence having at least 90%, preferably 95%, more preferably 97% or most preferably 99% sequence identity with an amino acid sequence shown in SEQ ID NO: 4. Preferably, the CD28 cytoplasmic domain is represented by an amino acid sequence shown in SEQ ID NO: 4. The CD28 cytoplasmic domain is a costimulatory domain and is derived from intracellular signaling domains of costimulatory molecules.

In an embodiment of the invention, the 4-1BB costimulatory domain is represented by an amino acid sequence having at least 90%, preferably 95%, more preferably 97% or most preferably 99% sequence identity with an amino acid sequence shown in SEQ ID NO: 25. Preferably, the 4-1BB costimulatory domain is represented by an amino acid sequence shown in SEQ ID NO: 25. Moreover, the 4-1BB costimulatory domain is represented by an nucleotide sequence having at least 90%, preferably 95%, more preferably 97% or most preferably 99% sequence identity with an nucleotide sequence shown in SEQ ID NO: 26. Preferably, the 4-1BB costimulatory domain is represented by an amino acid sequence shown in SEQ ID NO: 26.

In an embodiment of the invention, the CD3 zeta domain is represented by an amino acid sequence having at least 90%, preferably 95%, more preferably 97% or most preferably 99% sequence identity with an amino acid sequence shown in SEQ ID NO: 5. Preferably, the CD3 zeta domain is represented by an amino acid sequence shown in SEQ ID NO: 5. The CD3 zeta domain mediates downstream signaling during the T cell activation. It is derived from the intracellular signaling domain of the T cell receptor and contains ITAMs (immunoreceptor tyrosine based activation motifs).

In an embodiment of the invention, the extracellular domain comprises an amino acid sequence having at least 90%, preferably 95%, more preferably 97% or most preferably 99% sequence identity to an amino acid sequence shown in SEQ ID NO: 6. Preferably, the extracellular domain comprises an amino acid sequence shown in SEQ ID NO: 6. More preferably, the extracellular domain consists of an amino acid sequence shown in SEQ ID NO: 6.

In an embodiment of the invention, the intracellular signalling domain comprises an amino acid sequence having at least 90%, preferably 95%, more preferably 97% or most preferably 99% sequence identity to an amino acid sequence shown in SEQ ID NO: 7. Preferably, the intracellular signalling domain comprises an amino acid sequence shown in SEQ ID NO: 7. More preferably, the intracellular signalling domain consists of an amino acid sequence shown in SEQ ID NO: 7.

In a preferred embodiment of the invention, the SLAMF7-CAR polypeptide comprises an amino acid sequence having at least 90%, preferably 95%, more preferably 97% or most preferably 99% sequence identity to an amino acid sequence shown in SEQ ID NO: 8. Preferably, the SLAMF7-CAR polypeptide comprises an amino acid sequence shown in SEQ ID NO: 8. More preferably, the SLAMF7-CAR polypeptide consists of an amino acid sequence shown in SEQ ID NO: 8.

### Polynucleotide Encoding the SLAMF7-CAR Polypeptide

The present invention relates to a polynucleotide encoding the SLAMF7-CAR polypeptide of the present invention as defined above.

In an embodiment of the present invention, the polynucleotide encoding the SLAMF7-CAR polypeptide of the present invention is further flanked by a left and a right inverted repeat/direct repeat (IR/DR) segments. 11. The flanking segment in 5′-direction is represented by a left inverted repeat/direct repeat (IR/DR) segment and the flanking segment in 3′-direction is represented by a right inverted repeat/direct repeat (IR/DR) segment.

The nucleotide sequences of the left IR/DR segment and the nucleotide sequences of right IR/DR segment may be recognized by a Sleeping Beauty transposase protein. Preferably, the left IR/DR segment comprises a nucleotide sequence having at least 90%, preferably 95%, more preferably 97% or most preferably 99% sequence identity to the nucleotide sequence shown in SEQ ID NO: 9. Similarly, the right IR/DR segment comprises a nucleotide sequence having at least 90%, preferably 95%, more preferably 97% or most preferably 99% sequence identity to the nucleotide sequence shown in SEQ ID NO: 10.

The term “is flanked by” indicates that further nucleotides are present in the 5′-region and in the 3′-region of the polynucleotide sequence encoding the SLAMF7-CAR polypeptide which are all located on the same polynucleotide. Hence, the polynucleotide sequence encoding the SLAMF7-CAR polypeptide is flanked by IR/DR sequences, i.e. flanking segments, such that the presence of a transposase allows the integration of the polynucleotide encoding the SLAMF7-CAR polypeptide as well as the nucleotide sequences corresponding to the flanking segments into the genome of the transfected cell. In an aspect, the polynucleotide which is integrated into the genome comprises a polynucleotide encoding the SLAMF7-CAR polypeptide and a marker gene such as an EGFRt marker and is flanked by flanking segments. In this aspect, the region of the nucleotide sequence corresponding to the coding regions of the SLAMF7-CAR polypeptide and the EGFRt marker is considered to represent the reference segment.

When used in the present invention, the term “is flanked by” also means that the distance between a flanking segment and a reference segment to be less than 1000 bp, 900 bp, 800 bp, 700 bp, 600 bp, 500 bp, 400, 300 bp, 200 bp, 100 bp, 50 bp, 20 bp or less than 10 bp.

In this respect, the reference segment is the region corresponding to the coding region of the polynucleotides which are integrated into the genome. The overall architecture of the polynucleotide which is integrated into the genome of the transfected cell may be as follows (5′ to 3′ direction): [left IR/DR sequence]-[reference segment]-[right IR/DR sequence].

The distance between a flanking segment and a reference segment may be determined by counting the nucleotides between the 3′-end of the left IR/DR sequence and the 5′-end of the reference segment. Similarly, the distance between a flanking segment and a reference segment may be determined by counting the distance between the 3′-end of the reference segment and the 5′-end of the right IR/DR sequence. Both distances may be in the same such that the reference segment is centred between the flanking segments or the distances may be different.

The distance between the 3′-end of the left IR/DR sequence and the 5′-end of the reference segment may be less than 1000 bp, 900 bp, 800 bp, 700 bp, 600 bp, 500 bp, 400 bp, 300 bp, 200 bp or less than 100 bp.

The distance between the 3′-end of the reference segment and the 5′-end of the right IR/DR sequence may be less than 200 bp, 100 bp, 50 bp, 20 bp or less than 10 bp.

In an exemplary embodiment of the invention, the distance between the 3′-end of the left IR/DR sequence and the 5′-end of the reference segment may be less than 700 bp and the distance between the 3′-end of the reference segment and the 5′-end of the right IR/DR sequence may be less than 10 bp.

In an exemplary embodiment of the invention, the distance between the 3′-end of the left IR/DR sequence and the 5′-end of the reference segment may be less than 700 bp and more than 600 bp and the distance between the 3′-end of the reference segment and the 5′-end of the right IR/DR sequence may be less than 10 bp and more than 5 bp.

In an exemplary embodiment of the invention, the polynucleotide sequence encoding the SLAMF7-CAR and the EGFRt marker which is integrated into the genome of a transfected cell is represented by SEQ ID NO: 11.

In an embodiment of the present invention, the polynucleotide further comprises flanking segments in 5′-direction and in 3′-direction of the polynucleotide encoding the SLAMF7-CAR polypeptide. These flanking segments may relate to left IR/DR segments and to right IR/DR segments as described above.

In an embodiment of the present invention, the polynucleotide of the invention relates to a polynucleotide sequence comprising a nucleotide sequence of a left IR/DR segment, a polynucleotide sequence encoding the SLAMF7-CAR polypeptide and a nucleotide sequence of a right IR/DR segment. In an embodiment, the polynucleotide of the invention relates to a polynucleotide sequence having at least 90%, preferably 95%, more preferably 97% or most preferably 99% sequence identity to a nucleotide sequence shown in SEQ ID NO: 11. Preferably, the polynucleotide of the invention comprises a nucleotide sequence shown in SEQ ID NO: 11. More preferably, the polynucleotide of the invention consists of a nucleotide sequence shown in SEQ ID NO: 11.

### Expression Vector

The present invention relates to an expression vector comprising a polynucleotide of the present invention as defined above. A wide range of expression vectors for CARs are known in the art and are further detailed herein. For example, in some embodiments of the invention, the expression vector is a minimal DNA expression cassette. Moreover, an expression vector may be a DNA expression vector such as a plasmid, linear expression vector or an episome. In certain aspects, the vector comprises additional sequences, such as sequences that facilitate expression of the CAR, such as a promoter, enhancer, poly-A signal, and/or one or more introns. In certain aspects, the expression vector may be a transposon donor DNA molecule, preferably a minicircle DNA.

The present invention also relates to minicircle DNA comprising a polynucleotide of the present invention as defined above. As used herein, the term “minicircle DNA” refers to vectors which are supercoiled DNA molecules that lack a bacterial origin of replication and an antibiotic resistance gene. Therefore, they are primarily composed of a eukaryotic expression cassette.

In a useful embodiment the minicircle DNA of the invention is introduced into the cell in combination with mRNA encoding the SB transposase protein by electrotransfer, such as electroporation, nucleofection; chemotransfer with substances such as lipofectamin, fugene, calcium phosphate; nanoparticles, or any other conceivable method suitable to transfer material into a cell.

In an embodiment of the present invention, the minicircle DNA comprises the nucleotide sequence represented by SEQ ID NO: 12.

### Recombinant Immune Cell

The present invention also relates to a recombinant immune cell (preferably recombinant lymphocyte, more preferably recombinant T cell) comprising a polynucleotide of the present invention as defined above.

In an embodiment of the invention, the recombinant immune cell (preferably recombinant lymphocyte, more preferably recombinant T cell) relates to a recombinant immune cell wherein the polynucleotide as defined above is located on the nuclear genome of the immune cell.

In an embodiment of the invention, the recombinant immune cell (preferably recombinant lymphocyte, more preferably recombinant T cell) comprises the polynucleotide sequence of the invention which is flanked by left and right IR/DR sequences as described above on the nuclear genome due to integration using SB transposase. Hence, detection of a recombinant immune cell (preferably recombinant lymphocyte, more preferably recombinant T cell) comprising the polynucleotide of the invention is possible due to the presence of the IR/DR sequence which are flanking the polynucleotide encoding the SLAMF7-CAR polypeptide of the present invention on the nuclear genome. Thereby, the recombinant immune cell (preferably recombinant lymphocyte, more preferably recombinant T cell) of the present invention are structurally distinct from a recombinant immune cell obtained by viral based transfection methods.

The recombinant immune cell (preferably recombinant lymphocyte, more preferably recombinant T cell) is also capable of expressing the polynucleotide of the present invention. Thereby, the SLAMF7-CAR polypeptide which is encoded by the polynucleotide of the invention is translated and integrated into the cell membrane of the recombinant immune cell.

Expression of the SLAMF7 CAR polypeptide allows the recombinant immune cell (preferably recombinant lymphocyte, more preferably recombinant T cell) of the present invention to acquire specific reactivity against target cells expressing the SLAMF7 antigen, including MM cells. These SLAMF7 CAR-T cells are able to recognize and (antigen-specifically) eradicate MM cells. They are able to proliferate and to induce an immune response after encountering the SLAMF7 antigen.

In an embodiment of the present invention, the recombinant immune cell (preferably recombinant lymphocyte, more preferably recombinant T cell) relates to a recombinant CD4⁺ T cell or a recombinant CD8⁺ T cell. Preferably, the present invention relates to a plurality of recombinant T cells having a defined ratio of recombinant CD4⁺ T cells to recombinant CD8⁺ T cell. While CD8⁺ T cells are the key players in target cell eradication by cytolytic activity, the CD4⁺ T cells confer cytotoxic reactivity and influence the immune response by the release of cytokines. Hence, a plurality of recombinant T cell having a defined ratio of recombinant CD4⁺ T cells and recombinant CD8⁺ T cells may show improved properties compared to a plurality of recombinant T cells which are not provided in a defined ratio.

In an embodiment of the invention, the modified T cell of the present invention may further express the EGFRt marker on the cell surface. The EGFRt marker can be used to detect, track, select and deplete the modified T cell of the present invention. Therefore, analysis of drug product persistence following administration of the modified T cell is made available. Furthermore, the EGFRt marker makes modified T cells of the invention sensitive to ADCC/CDC through the antibody Cetuximab which can therefore be used as safety switch.

The amino acid sequence of the EGFRt which may be used in the present invention is represented by SEQ ID NO: 15.

In an embodiment of the invention, the recombinant immune cell is obtained from an immune cell (preferably lymphocyte, more preferably T cell) derived from a mammal, preferably a human.

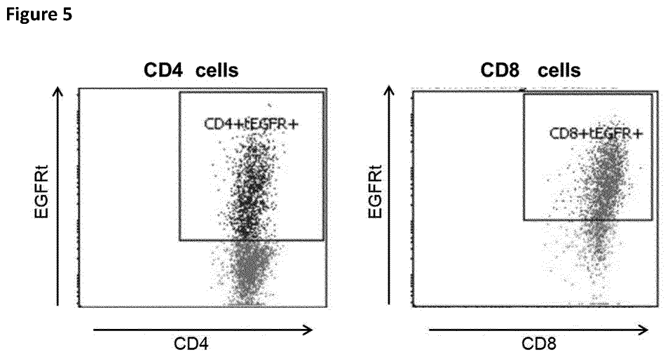

In a preferred embodiment of the invention, the recombinant immune cells (preferably recombinant lymphocytes, more preferably recombinant T cells) may be formulated in infusion solution (0.45% NaCl plus 2.5% glucose plus 1% human serum albumin) at a final concentration of 1×10⁴, 3×10⁴, 1×10⁵, 3×10⁵, 1×10⁶, 3×10⁷, 1×10⁸, 3×10⁸, 1×10⁹ or 3×10⁹ cells/mL (the volume in mL corresponds to the weight in kg) and filled in infusion bags. CAR-positive CD4⁺ and CD8⁺ cells may be formulated as close as technically possible to a 1:1 ratio (range 0.5-2:1). Since usually not all T cells are gene-modified, the formulation may also include unmodified CD4⁺ and CD8⁺ T cells (up to 90%, FIG. 5). Unmodified T cells are not considered for calculating the dose of the formulation. The total amount of CD4⁺ and CD8⁺ T cells (gene-modified and unmodified) may not be equally high.

In an embodiment of the present invention, the recombinant immune cells (preferably recombinant lymphocytes, more preferably recombinant T cells) do not comprise an amino acid sequence of the SB transposase as represented by SEQ ID NO: 13 or fragments thereof in a detectable amount at day 14 after gene transfer. The detectable amount at day 14 after gene transfer may be determined as detailed in the experimental section (see Residual transposase, FIG. 11, 12).

### Method for Producing Recombinant Immune Cells

The present invention also relates to a method for producing recombinant immune cells (preferably recombinant lymphocyte, more preferably recombinant T cell) of the present invention as defined above.

In an embodiment of the present invention, the method for producing recombinant immune cells comprises the steps of (a) isolating immune cells from a blood sample of a subject, (b) transforming immune cells using a transposable element comprising a polynucleotide as described above and a Sleeping Beauty (SB) transposase to produce recombinant immune cells followed by (c) purifying immune cells.

In an embodiment of the present invention, the immune cells are lymphocytes, more preferably T cells.

In a further embodiment, the T cell is a CD4⁺ T cell and/or a CD8⁺ T cell.

In a further embodiment of the invention, the recombinant CD4⁺ T cells and recombinant CD8⁺ T cells may be expanded separately.

In a further embodiment of the invention, the blood sample is derived from a human subject, preferably a human subject diagnosed with cancer, preferably diagnosed with multiple myeloma.

In a further embodiment of the invention, plurality of recombinant CD4⁺ T cells and a plurality of recombinant CD8⁺ T cells are combined in a defined ratio to form a composition of recombinant T cells, wherein the ratio of said recombinant T cells in the composition is in the range of 0.5:1 to 2:1.

In a preferred embodiment of the invention, the method for producing recombinant immune cells (preferably recombinant lymphocyte, more preferably recombinant T cell) provides a formulation comprising recombinant immune cells (preferably recombinant lymphocyte, more preferably recombinant T cell) in an infusion solution (0.45% NaCl plus 2.5% glucose plus 1% human serum albumin) at a final concentration of 1×10⁴, 3×10⁴, 1×10⁵, 3×10⁵, 1×10⁶ , 3×10⁶, 1×10⁷, 3×10⁷, 1×10⁸, 3×10⁸, 1×10⁹ or 3×10⁹ cells/mL (the volume in mL corresponds to the weight in kg) and filled in infusion bags. An infusion solution of 1000 ml may generally comprise 4.5 g NaCl and 27.5 g glucose-monohydrate (Ph. Eur.) and water. The recombinant CD4⁺ and CD8⁺ T cells are preferably formulated as close as technically possible to a 1:1 ratio (range 0.5-2:1). Since usually not all T cells are gene-modified, the formulation may also include unmodified CD4⁺ and CD8⁺ T cells (up to 90%, FIG. 5). Unmodified T cells are not considered for calculating the dose of the formulation. The total amount of CD4⁺ and CD8⁺ T cells (gene-modified and unmodified) may not be equally high.

Moreover, the method for producing recombinant immune cells (preferably recombinant lymphocyte, more preferably recombinant T cell) of the present invention may essentially consists of following the steps (see also FIG. 4):
isolation of CD8⁺ and CD4⁺ T cells from patient blood and stimulation with anti-CD3/anti-CD28 dynabeads and Interleukin-2
gene modification of T cells to express the SLAMF7 CAR polypeptide of the invention using mRNA encoding for SB100X transposase and minicircle DNA as described above
expansion of CD8⁺ and CD4⁺ T cells in separate cultures
formulation of the SLAMF7 CAR-T cell product at a defined CD4:CD8 ratio (1:1 ratio, range 0.5-2:1).

Stability of genomically integrated transgenes is of paramount importance for clinical applications. That is, ideally, transposition should take place only once during transfer of the therapeutic transgene from the MC into the cellular genome. Any further transposition event between chromosomal locations is undesired. One major determinant of potentially on-going transposition events in cell populations is the prolonged availability of the transposase. For this reason, the SB transposase is being supplied in form of transiently stable messenger ribonucleic acid (mRNA). This has the advantage of clearance of both the mRNA and the encoded transposase protein in the 14-day expansion period of CAR-T cell manufacturing. Importantly, the half-life of SB transposase has been estimated to be ^{˜}80 h in cycloheximide-treated cultured cells ([9]).

The SB transposase which may be used in the present invention is represented by an amino acid sequence shown in SEQ ID NO: 13.

The invention also relates to a recombinant immune cell (preferably recombinant lymphocyte, more preferably recombinant T cell) or a formulation of recombinant immune cells (preferably recombinant lymphocytes, more preferably recombinant T cells) obtainable by the method as described above.

### Pharmaceutical Composition

The present invention also relates to a pharmaceutical composition comprising a plurality of recombinant immune cells (preferably recombinant lymphocyte, more preferably recombinant T cell) as described above.

In an embodiment of the invention, the pharmaceutical composition comprises recombinant CD4⁺ T cells and recombinant CD8⁺ T cells both comprising the polynucleotide of the present invention and both expressing the SLAMF7 CAR polypeptide. The pharmaceutical composition of the invention comprises recombinant CD4⁺ T cells and recombinant CD8⁺ T cells in a defined ratio of 0.5-2.1, preferably in a range of 0.75-1.5, more preferably in a range pf 0.8-1.3, even more preferably in a range of 0.9-1.2 and most preferably in a ratio of 1:1.

In a further embodiment of the invention, the pharmaceutical composition may be formulated as infusion solution comprising NaCl, glucose and human serum albumin in an amount of 0.45%, 2,5% and 1%, respectively.

### Pharmaceutical Composition for Use as a Medicament

The present invention also relates to a pharmaceutical composition as described above for use as a medicament.

In an embodiment of the invention, the pharmaceutical composition as described above is used in a method of treating cancer, wherein in said method the pharmaceutical composition of the present invention is to be administered to a subject.

In an embodiment of the invention, the pharmaceutical composition as described above is to be administered in a dose of about 1×10⁴ cells/kg body weight, of about 3×10⁴ cells/kg body weight, of about 1×10⁵ cells/kg body weight, of about 3×10⁵ cells/kg body weight, of about 1×10⁶ cells/kg body weight, or of about 3×10⁶ cells/kg body weight, of about 1×10⁷ cells/kg body weight, of about 3×10⁷ cells/kg body weight, of about 1×10⁸ cells/kg body weight, of about 3×10⁸ cells/kg body weight, of about 1×10⁹ cells/kg body weight, or of about 3×10⁹ cells/kg body weight. In an embodiment of the invention, the pharmaceutical composition as described above is to be administered in a dose of about 1×10⁶ to 1×10⁹ cells. The pharmaceutical composition is to be administered in a single dose or in multiple doses.

The term “about” used in the context of the present invention means that the value following the term “about” may vary within the range of +/−20%, preferably in the range of +/−15%, more preferably in the range of +/−10%.

In an embodiment of the invention, the pharmaceutical composition is to be administered intravenously.

In an embodiment of the invention, the pharmaceutical composition as described above comprises a plurality of recombinant CD4⁺ T cells and CD8⁺ T cells in a defined ratio, wherein the ration is in the range of 0.5:1 to 2:1, preferably in the range of 0.75:1 to 1.5:1, more preferably in the range of 0.8:1 to 1.3:1, even more preferably in the range of 0.9:1 to 1.2:1 and most preferably the ratio is 1:1.

In an embodiment of the invention, the pharmaceutical composition as described above is used to treat cancer in a human subject, wherein the cancer is caused by abnormal cells expressing and displaying the SLAMF7 protein. Preferably, the cancer is selected from the group consisting of multiple myeloma, T-cell leukemia or -lymphoma, B-cell leukemia or—lymphoma, preferably multiple myeloma. Further diseases which may also be treated using the pharmaceutical composition of the invention are Monoclonal gammopathy of undetermined significance (MGUS) or Smouldering multiple myeloma (SMM).

Moreover, the pharmaceutical composition as described above for use as a medicament is used in the treatment of antibody-mediated autoimmune diseases such as Graves' disease, myasthenia gravis, lupus erythematosus, rheumatoid arthritis, goodpasture syndrome, scleroderma, CREST syndrome, granulomatosis with polyangiitis, microscopic polyangiitis, pemphigus vulgaris, Sjögren's syndrome, diabetes mellitus type 1, primary biliary cholangitis, Hashimoto's thyreoiditis, neuromyelitis optica spectrum disorders, anti-NMDA receptor encephalitis, vasculitis or multiple sclerosis.

In a preferred embodiment, the pharmaceutical composition comprising recombinant immune cells (preferably recombinant lymphocyte, more preferably recombinant T cell) is formulated in infusion solution (0.45% NaCl plus 2.5% glucose plus 1% human serum albumin) at a final concentration of 1×10⁴, 3×10⁴, 1×10⁵, 3×10⁵, 1×10⁶, 3×10⁶, 1×10⁷, 3×10⁷, 1×10⁸, 3×10⁸, 1×10⁹ or 3×10⁹ cells/mL (the volume in mL corresponds to the weight in kg) and filled in infusion bags. The CAR-positive CD4⁺ and CD8⁺ cells may be formulated as close as technically possible to a 1:1 ratio (range 0.5:1 to 2:1). Since usually not all T cells are gene-modified, the formulation may also include unmodified CD4⁺ and CD8⁺ T cells (up to 90%, FIG. 5). Unmodified T cells are not considered for calculating the dose of the formulation. The total amount of CD4⁺ and CD8⁺ T cells (gene-modified and unmodified) may not be equally high.

The pharmaceutical composition as described above comprising the modified T cells are stored at 2-8° C. The pharmaceutical composition is stable for (at least) 48 hours after formulation and ought to be administered to the patient within this period.

### Safety

Potential risks which were associated with CAR-T cell products include CRS, immune cell-associated neurotoxicity (ICANS), infusion reactions, risks associated with lymphodepleting, fever, allergic reactions and tumor lysis syndrome (TLS), on-target-off-tumor toxicities, infectious diseases, insertional oncogenesis, secondary malignancies, febrile neutropenia.

### Cytokine Release Syndrome (CRS)

Cytokine release syndrome is characterized by a series of inflammatory symptoms resulting from cytokine elevations. It is triggered by the activation of CAR-T cells on engagement with their specific antigens. The activated T cells release cytokines and chemokines, as do bystander immune cells such as monocytes and/or macrophages.

In most patients, CRS symptoms are mild and flulike, with fever and myalgia. However, some patients experience a severe inflammatory syndrome that includes vascular leakage, hypotension, pulmonary edema, and coagulopathy, resulting in multi-organ system failure and death. In the study by Maude et al., severe cytokine release started a median of one day after infusion, whereas non-severe CRS started 4 days later ([10]).

A consensus grading system for CRS due to T cell therapies was developed by the American Society for Transplantation and Cellular Therapy (ASBMT, [11]).

CRS can be managed by targeting IL-6 without evidence of therewith compromising the clinical efficacy of T cell therapies. Tocilizumab a recombinant humanized monoclonal antibody that blocks IL-6 from binding to its receptor was approved by the FDA in 2017 and the EMA in 2018 to treat severe or life-threatening CAR-T cell-induced CRS in adults and pediatric patients 2 years of age and older. In a retrospective pooled analysis including 45 pediatric and adult patients treated with tocilizumab for severe or life-threatening CRS, with or without additional high-dose corticosteroids, 31 patients (69%; 95% CI: 53%-82%) achieved a response, defined as resolution of CRS within 14 days of the first dose of tocilizumab, no more than two doses of tocilizumab were needed, and no drugs other than tocilizumab and corticosteroids were used for treatment ([12]). In patients who respond to tocilizumab, fever and hypotension often resolve within a few hours, and vasopressors and other supportive care measures can be weaned quickly thereafter ([13]; [10]). In some cases, symptoms may however not resolve completely, and continued aggressive supportive therapy may be necessary for several days, along with the administration of a second dose of tocilizumab and/or a second immunosuppressive agent such as corticosteroids ([14]).

### Immune Cell-Associated Neurotoxicity Syndrome (ICANS)

Neurologic toxicities including confusion, delirium, expressive aphasia, obtundation, myoclonus, and seizure were reported in patients receiving CAR-T cells ([10], [15], [16]).

The pathophysiology of these neurologic side effects is unknown although it is plausible that elevated cytokine levels are partly responsible. Conversely, direct CAR-T cell toxicity on the central nervous system is possible but has not been demonstrated. Neurological events may occur at different times than CRS or in the absence of CRS toxicities ([10]), suggesting that neurologic toxicity might have a different mechanism than other toxicities such as fever and hypotension.

In most instances, neurologic events are self-limiting, and there are no definitive consensus guidelines regarding best management of these events. It is unclear if tocilizumab has any beneficial effect. Because tocilizumab is a monoclonal antibody, its size makes efficient Blood-Brain Barrier (BBB) penetration unlikely. The smaller IL-6 molecule crosses the BBB and has been shown to cause neurologic defects. Saturation of IL-6 receptors following systemic tocilizumab administration may increase serum IL-6 levels, theoretically increasing cerebrospinal fluid IL-6 levels that might worsen neurologic toxicity. As for other groups ([14]), the Transplantation and Immunology Branch of the US National Cancer Institute treats severe neurologic toxicities with systemic corticosteroids rather than tocilizumab as the first-line agent ([17]).

### Hemophagocytic Lymphohistiocytosis/Macrophage Activation Syndrome (HLH/MAS)

In the context of CAR-T cell therapy, HLH/MAS is a potentially serious disorder associated with uncontrolled activation and proliferation of CAR-T cells and subsequent activation of macrophages. The mechanism of post-CAR-T cell HLH/MAS is not well understood, and this form of secondary HLH/MAS may represent the most severe progression of CRS. Clinical presentation is characterized by high-grade, non-remitting fever, cytopenias, and hepatosplenomegaly. Laboratory abnormalities include elevated inflammatory cytokine levels, serum ferritin, soluble IL-2 receptor (sCD25), triglycerides, and decreased circulating NK cells. Other findings include variable levels of transaminases, signs of acute liver failure, coagulopathy, and disseminated intravascular coagulopathy. Diagnostic criteria for CAR-T cell related HLH/MAS have been proposed. To fulfill these criteria, an elevated ferritin of >10,000 ng/ml is required, along with at least two organ toxicities, including presence of hemophagocytosis in bone marrow or organs, or at least grade 3 transaminitis, renal insufficiency, or pulmonary edema ([18]). While there is considerable overlap in clinical manifestations and laboratory findings between HLH/MAS and CRS, other distinguishing HLH/MAS physical findings such as hepatosplenomegaly and lymphadenopathy are not common in adult patients treated with activated T cell therapies.

### Infusion Reactions/Hypersensitivity

Administration of SLAMF7 CAR-T cells may cause infusion reactions, such as fever, chills, rash, urticaria, dyspnea, hypotension, and/or nausea.

### Tumor Lysis Syndrome (TLS)

Although chemotherapy may have caused TLS in some cases, the infusion of CAR-T cells in the absence of prior conditioning chemotherapy has led to TLS ([19]; [13]). TLS is the result of rapid tumor cell lysis with subsequent release of intracellular metabolites into the blood, causing hyperuricemia, hyperkalemia, hyperphosphatemia and hypocalcemia. Eventually, TLS can induce acute kidney failure and life-threatening emergencies. As the amount of eliminated tumor cells correlates with CAR-T cell efficacy, TLS can coincide with CRS and appropriate management of TLS is relevant for optimized outcome in CAR-T cell therapy.

### Fever/Febrile Neutropenia

Patients who received SLAMF7 CAR-T cells might develop fever due to CRS (see section 6.2.1). Patients should be monitored closely for hemodynamic instability and changing neurologic status. Febrile subjects, neutropenic or otherwise, should be evaluated promptly for infection and managed per institutional or standard clinical practice.

### On-Target Off-Tumor Recognition/Lymphopenia

The ideal target antigen is restricted to the tumor cell. Unfortunately, most targets of CAR-T cells have shared expression on normal tissues and some degree of “on-target off-tumor” toxicity occurs through engagement of target antigen on nonpathogenic tissues. SLAMF7 has a low level of expression on normal cells, including T cells and NK cells and non-clinical data indicate that a moderate lymphoreduction must be expected after SLAMF7 CAR-T cell administration. This on-target off-tumor cytotoxic effect on autologous lymphocytes was also observed after treating myeloma patients with the huLuc63 antibody Elotuzumab. In the ELOQUENT-2 study, a stronger effect in lymphocyte reduction after the initial infusion was reported in the Elotuzumab group compared to the control group (77% versus 49%).

During the course of treatment absolute lymphocyte counts from patients of the Elotuzumab group stabilized at a slightly lower level compared to baseline and the control group. No significant increase in the infection rate in the Elotuzumab group was observed compared to the control group ([20]). No binding of the huLuc63 antibody to CD34⁺ hematopoietic stem cells was reported. Given the potential for off-tumor toxicity of SLAMF7 CAR-T cells, patients should be closely monitored for lymphocyte levels and infections.

### Infectious Diseases

Patients on CAR-T cell clinical trials frequently become neutropenic and lymphopenic after administration of chemotherapy followed by CAR-T cells, predisposing them to opportunistic infections and/or infectious reactivations. In this setting, signs including elevated body temperature, tachycardia, and hypotension associated with CRS can be difficult to differentiate from septicaemia. In an early report, a patient with chronic lymphocytic leukemia who received chemotherapy and CD19 CAR-T cells died with fever, hypotension, and renal failure. It was later found that this patient had elevated serum levels of inflammatory cytokines before CAR-T cell infusion, suggesting that the patient had a prior infection ([21]). Bacteremias of various foci (e.g. Salmonella, urinary tract infections) and viral infections (incl. influenza, respiratory syncytial virus, and herpes zoster virus) have been observed following CAR-T cell infusion. As SLAMF7 CAR-T cells might further induce lymphoreduction, patients should be carefully monitored for infectious complications.

### Insertional Oncogenesis/Uncontrolled T Cell Proliferation

Insertion of a transgene into differentiated T cells carries the risk of induced malignant transformation. However, no adverse or toxic events related to the gene transfer procedure have been reported to date. Accordingly, no genotoxic effect of integrating vectors, nor clonal dominance of gene modified T cells has been observed ([22]; [23]). By using SB transposition for gene transfer the risk of insertional oncogenesis is even reduced. Analysis of the genomic insertion sites and copy numbers in DP cells revealed a safer integration profile in comparison to viral vectors ([24]). In none of the mice experiments which were conducted, uncontrolled T cell proliferation was observed.

In non-clinical studies and clinical experience ([13], [10]), CD19 CAR-Transduced cells have only proliferated in response to physiologic signals or upon exposure to CD19 antigen. In the context of SLAMF7 CAR-T cell therapy, it is expected that the T cells will proliferate in response to signals from the SLAMF7 expressing malignant tumor and normal lymphocytes.

### Secondary Malignancies

Any treatment with cytostatic agents can potentially increase the risk of secondary malignancies. For details, please refer to the summary of product characteristics for fludarabine phosphate and cyclophosphamide. To date, it is unclear whether patients treated with SLAMF7 CAR-T cells develop secondary malignancies due to the adoptive transfer. Therefore, a respective long-term follow-up is put in place.

### Off-Target Antigen Recognition

Most CAR-T cells recognize antigen through scFv derived from monoclonal antibodies, some of which may have a proven safety record in clinical use. Organ damage could hypothetically occur when CAR-T cells cross-react with antigens expressed on normal tissue that are similar to the target antigen expressed by the malignancy. This toxicity has not been documented in clinical trials of CARs but has been observed in clinical trials of T cells genetically modified to express T cell receptors ([25]). The SLAMF7 CAR is derived from the huLuc63 antibody Elotuzumab, which is already used for MM treatment. No off-target antigen recognition has been reported for this antibody.

Risks Associated with Lymphodepleting Chemotherapy

Patients will receive fludarabine and cyclophosphamide five to two days prior to treatment with SLAMF7 CAR-T cells. Refer to the summary of product characteristics for specific details surrounding the risks of fludarabine phosphate and cyclophosphamide.

### Previous and Concomitant Medication and Procedures

All medications taken within 8 weeks prior to the day of leukapheresis are defined as previous medications. Concomitant medications are all medications given during the clinical trial starting at or after the day of leukapheresis. They must be listed in the patient's medical record and documented in the corresponding section of the eCRF.

### Permitted and Prohibited Previous Medications

In principle, anti-myeloma therapy is permitted in the time period between study enrolment and leukapheresis, in order to prevent massive myeloma progression and deterioration of the study patient which may preclude performing the leukapheresis.

A preferred anti-myeloma therapy may include e.g. Bortezomib, Revlimid and Dexamethason; or Carfilzomib, Revlimid and Dexamethasone. Anti-myeloma agents that are myelosuppressive ought to be avoided.

However, the following treatments are not allowed within 8 weeks prior to the scheduled date of leukapheresis:
Anti-CD38 and anti-SLAMF7 antibodies
Anti-PD1, anti-PD-L1 and anti-CTLA4 antibodies

Also, the following treatments are not allowed within 4 weeks prior to the scheduled date of leukapheresis:
Cyclophosphamide and Bendamustine
Any other investigational agent or investigational cellular therapy

Also, the following treatments are not allowed within 1 week prior to the scheduled date of leukapheresis:
Immunomodulatory agents (e.g. IMiDs)
Systemic steroids (NB: Physiologic replacement and topical steroids are permitted)

### Bridging Therapy

Bridging therapy may be administered during the manufacturing process of the SLAMF7 CAR-T product. The aim is to prevent massive disease progression, deterioration of organ function or other complications, which will interfere or prevent lymphodepletion and infusion of the SLAMF7 CAR-T product. The bridging therapy is permitted in the time interval after completion of leukapheresis and prior to LD therapy.

A preferred treatment regimen for bridging therapy may include Bortezomib, Revlimid and Dexamethason; or Carfilzomib, Revlimid and Dexamethasone.

The following therapies are not allowed:
Anti-CD38 and anti-SLAMF7 antibodies
Anti-PD1, anti-PD-L1 and anti-CTLA4 antibodies
Any other investigational agent or investigational cellular therapy

### Prohibited and Restricted Concomitant Medications and Procedures

The following medications are prohibited or restricted during CAR-T cell infusion and thereafter:

Steroids: dexamethasone, prednisone or other corticosteroids are not allowed. If steroids are to be administered, it should be discussed with the medical monitor unless in the setting of acute clinical requirements (e.g. CRS, ICANS, life-threatening conditions). Generally, the only setting for administration of corticosteroids will be CRS management or severe neurotoxicity, following the guidelines in Section.

Pretreatment containing steroids may be given for necessary medications (e.g. intravenous immunoglobulins) after discussion with the sponsor. Premedication with steroids for SLAMF7 CAR-T infusion is not allowed. Physiologic replacement dosing of steroids (≤12 mg/m²/day hydrocortisone or equivalent [≤3 mg/m²/day prednisone or ≤0.45 mg/m²/day dexamethasone]) is allowed. Topical steroids, inhaled steroids, and intrathecal steroids for central nervous system (CNS) relapse prophylaxis are permitted.

Any chemotherapy, radiation therapy, immunotherapy, biologic or hormonal therapy for treatment of MM prior to documentation of PD (palliative radiotherapy for treatment of symptomatic bone or soft tissue lesions is allowed—but must be notified to the sponsor).

### Herbal and Natural Remedies.

The following medications should be used with caution during the trial. The sponsor must be notified if a patient receives any of these during the trial:

Any biologic or hormonal therapy. Of note: concurrent use of hormones for non-cancer-related conditions (e.g. insulin for diabetes and hormone replacement therapy) is acceptable;

Immunosuppressive medications including, but not limited to, systemic corticosteroids at doses not exceeding 10 mg/day of prednisone or equivalent, methotrexate, azathioprine, and tumor necrosis factor alpha (TNF-α) blockers.

Note: Use of immunosuppressive medications in patients with allergies to contrast agents is acceptable in principle however, if a patient has a known allergy, imaging ought to be done without contrast agents.

### Allowed Concomitant Medication and Procedures

The following concomitant medications and procedures will be allowed during the trial if clinical indicated:
Patients with serum IgG level less than 400 mg/dL will receive intravenous immunoglobulin replacement as needed to maintain an IgG level above 400 mg/dL if the patient has had one or more infections when not neutropenic while enrolled on the protocol.
Patients with a history of herpes simplex virus infection should receive herpes simplex virus prophylaxis at the discretion of the investigator.
Patients with a CD4 T cell count of <200 will be maintained on pneumocystis prophylaxis with trimethoprim-sulfamethoxazole 1 double-strength tablet every Monday-Wednesday-Friday. Folic acid supplementation will be added. If patients cannot tolerate trimethoprim sulfamethoxazole, an alternative pneumocystis prophylaxis will be used.
In the absence of contraindications, all patients should receive antibacterial (e.g. levofloxacin) and antifungal (e.g. fluconazole) infection prophylaxis during neutropenia (absolute neutrophil count [ANC]<500/μl).
Fevers in the presence of neutropenia should be managed according to local institutional guidelines with regards to broad spectrum antibiotics and management.
Transfusion support of irradiated platelets and packed red blood cells (RBCS) may be used at the discretion of the treating investigator. Leukocyte filters are encouraged for all platelet and packed RBC transfusions.

Patients with history of seizures should consider use of levetiracetam as seizure prophylaxis.

In general, anti-coagulants are allowed in patients that require systemic anti-coagulation and are on a stable dose of anti-coagulants.

### EXAMPLES

SLAMF7 CAR-T cell as generated in the experimental section of the application relates to an exemplified embodiment of the modified T cell of the present invention.

### Generation of Minicircle DNA

Minicircle DNA is manufactured, filled and stored as an independent batch.

Minicircle DNA has been manufactured in a process size that resulted in a 5 mg final product batch size. 0.2 μm filtration is conducted under a laminar air flow hood.

The process starts with a glycerol cell bank (RCB) carrying the parental plasmid (PP), which is amplified by fermentation and a recombination is induced by the addition of an inducer (L-arabinose). This leads to the expression of a recombinase that causes the cis-recombination of the two recombination sequences flanking the minicircle sequence on the PP and leading to the generation of the minicircle. The minicircle DNA is purified subsequently to be obtained in a pure and supercoiled form. Methods for generating minicircle DNA as used in the present invention are commonly available in the art as described e.g. in [36], [37].

### Characterization

Minicircles (MC) are supercoiled DNA vectors that constitute an alternative to plasmids as source of SB-transposase and transposon. MCs are minimal expression cassettes devoid of bacterial origins of replication and antibiotic resistance or other selection marker genes, and derived from conventional plasmids in this case carrying a kanamycine resistance marker gene through an intramolecular recombination step during propagation in Escherichia coli.

The minicircle DNA shown in Table 1 below used in the manufacturing of SLAMF/CAR-T cells comprises the following elements:
SCAR: Minimal remaining sequences deriving from cloning steps (no coding function) and recombination sequence
Left/right IR: Left and right inverted repeats which are recognized and bound by transposase
EF-1alpha core promoter: Core promoter of human elongation factor EF-1alpha
Kozak: Kozak sequence (involved in translation initiation)
Signal peptide: GM-CSF signal peptide
huLuc63 VH: heavy chain of variable fragment (derived from SLAMF7-binding antibody elotuzumab, also known as huLuc63)
Linker: (4GS) 3 linker element
huLuc63 VL: light chain of variable fragment (derived from SLAMF7-binding antibody elotuzumab)
IgG4-Fc Hinge: IgG4-Fc long spacer (Hinge-CH2-CH3, with 4/2NQ modification)
CD28 tm: CD28 transmembrane domain
CD28 cytoplasmic: CD28 cytoplasmatic (co-stimulatory) domain
CD3 zeta: CD3-zeta domain
T2A: ribosomal skip element to separate CAR and EGFRt
Signal peptide: GM-CSF signal peptide
EGFRt: truncated EGFR tag to facilitate testing for CAR⁺ cells and as potential depletion marker (suicide switch)

TABLE 1
Nucleotide sequence of the SLAMF7 CAR in minicircle (SEQ ID NO: 12)

| | |
|---|---|
| 1 | gccttggtca aattgggtat acccatttgg gcctagtcta gccggcatgg cgcattacag |
| 61 | caatacgcaa tttaaatgcg cctagcgcat tttcccgacc ttaatgcgcc tcgcgctgta |
| 121 | gcctcacgcc cacatatgtg cagatctgaa gaagaagaag aagaagaaga agaagaagaa |
| 181 | gaagaagaag aagaagaaga agaagaagaa gtttctcgga tccctataca gttgaagtcg |
| 241 | gaagtttaca tacacttaag ttggagtcat taaaactcgt ttttcaacta ctccacaaat |
| 301 | ttcttgttaa caaacaatag ttttggcaag tcagttagga catctacttt gtgcatgaca |
| 361 | caagtcattt ttccaacaat tgtttacaga cagattattt cacttataat tcactgtatc |
| 421 | acaattccag tgggtcagaa gtttacatac actaagttga ctgtgccttt aaacagcttg |
| 481 | gaaaattcca gaaaatgatg tcatggcttt agaagcttga tatccatgga attcggatct |
| 541 | gcgatcgctc cggtgcccgt cagtgggcag agcgcacatc gcccacagtc cccgagaagt |
| 601 | tggggggagg ggtcggcaat tgaaccggtg cctagagaag gtggcgcggg gtaaactggg |
| 661 | aaagtgatgt cgtgtactgg ctccgccttt ttcccgaggg tgggggagaa ccgtatataa |
| 721 | gtgcagtagt cgccgtgaac gttctttttc gcaacgggtt tgccgccaga acacagctga |
| 781 | agcttcgagg ggctcgcatc tctccttcac gcgcccgccg ccctacctga ggccgccatc |
| 841 | cacgccggtt gagtcgcgtt ctgccgcctc ccgcctgtgg tgcctcctga actgcgtccg |
| 901 | ccgtctaggt aagtttaaag ctcaggtcga gaccgggcct ttgtccggcg ctcccttgga |
| 961 | gcctacctag actcagccgg ctctccacgc tttgcctgac cctgcttgct caactctacg |
| 1021 | tctttgtttc gttttctgtt ctgcgccgtt acagatccaa gctgtgaccg gcgcctacgg |
| 1081 | ctagcgccgc caccatgctg ctgctcgtga catctctgct gctgtgcgag ctgccccacc |
| 1141 | ccgcctttct gctgattcct gaggtgcagc tggtggaaag cggcggagga ctggtgcagc |
| 1201 | ctggcggatc tctgagactg agctgtgccg ccagcggctt cgacttcagc cggtactgga |
| 1261 | tgagctgggt gcgccaggcc cctggcaaag gcctggaatg gatcggcgag atcaaccccg |
| 1321 | acagcagcac catcaactac gcccccagcc tgaaggacaa gttcatcatc agccgggaca |
| 1381 | acgccaagaa cagcctgtac ctgcagatga actccctgcg ggccgaggac accgccgtgt |
| 1441 | actattgcgc cagacccgac ggcaactact ggtacttcga cgtgtggggc cagggcaccc |
| 1501 | tcgtgacagt gtctagcgga ggcggcggat ctggcggagg gggatctggg ggcggaggct |
| 1561 | ctgatatcca gatgacccag agccccagca gcctgtctgc cagcgtgggc gacagagtga |
| 1621 | ccatcacatg caaggccagc caggacgtgg gaatcgccgt ggcctggtat cagcagaaac |
| 1681 | ccggcaaggt gcccaagctg ctgatctact gggccagcac cagacacacc ggcgtgcccg |
| 1741 | atagattttc cggcagcggc tccggcaccg acttcaccct gacaatcagc tccctgcagc |
| 1801 | ctgaggacgt ggccacctac tactgccagc agtacagcag ctacccctac accttcggac |
| 1861 | agggcaccaa ggtggaaatc aaagagtcta agtacggacc gccttgtcct ccttgtccag |
| 1921 | ctcctcctgt ggccggacct agcgtgttcc tgttcccccc aaagcccaag gacaccctga |
| 1981 | tgatcagccg gacccccgaa gtgacctgcg tggtggtgga tgtgtcccag gaagatcccg |
| 2041 | aggtgcagtt caattggtac gtggacggcg tggaagtgca caacgccaag accaagccca |
| 2101 | gagaggaaca gttccagagc acctaccggg tggtgtccgt gctgacagtg ctgcaccagg |
| 2161 | actggctgaa cggcaaagag tacaagtgca aggtgtccaa caagggcctg cccagcagca |
| 2221 | tcgagaaaac catcagcaag gccaagggcc agcctcgcga gccccaggtg tacacactgc |
| 2281 | ctccaagcca ggaagagatg accaagaacc aggtgtccct gacctgtctc gtgaagggct |
| 2341 | tctaccccag cgacattgcc gtggaatggg agagcaacgg ccagcccgag aacaactaca |
| 2401 | agaccacccc ccctgtgctg gacagcgacg gctcattctt cctgtacagc agactgaccg |
| 2461 | tggacaagag ccggtggcag gaaggcaacg tgttcagctg cagcgtgatg cacgaggccc |
| 2521 | tgcacaacca ctacacccag aagtccctgt ctctgagcct gggcaagatg ttctgggtgc |
| 2581 | tggtggtcgt gggcggagtg ctggcctgtt acagcctgct cgtgaccgtg gccttcatca |
| 2641 | tcttttgggt gcgcagcaag cggagcagag gcggccacag cgactacatg aacatgaccc |
| 2701 | ccagacggcc tggccccacc agaaagcact accagcctta cgcccctccc agagacttcg |
| 2761 | ccgcctaccg gtccagagtg aagttcagca gaagcgccga cgcccctgcc tatcagcagg |
| 2821 | gccagaacca gctgtacaac gagctgaacc tgggcagacg ggaagagtac gacgtgctgg |
| 2881 | ataagcggag aggccgggac cctgagatgg gcggcaagcc tagaagaaag aacccccagg |
| 2941 | aaggcctgta taacgaactg cagaaagaca agatggccga ggcctacagc gagatcggca |
| 3001 | tgaagggcga aagaaggcgg ggcaagggcc acgatggcct gtatcaggga ctgagcaccg |
| 3061 | ccaccaagga tacctatgac gcactgcaca tgcaggccct gccccccaga ctcgagggcg |
| 3121 | gaggcgaagg cagaggatct ctgctgacat gcggcgacgt ggaagagaac ccaggcccca |
| 3181 | gaatgctgct gctcgtgaca agcctgctgc tgtgcgagct gccccaccct gcctttctgc |
| 3241 | tgatcccccg gaaagtgtgc aacggcatcg gcatcggaga gttcaaggac agcctgtcca |
| 3301 | tcaacgccac caacatcaag cacttcaaga attgcaccag catcagcggc gacctgcaca |
| 3361 | tcctgccagt ggcctttaga ggcgacagct tcacccacac ccccccactg gatccacagg |
| 3421 | aactggatat tctgaaaacc gtaaaggaaa tcacagggtt tttgctgatt caggcttggc |
| 3481 | ctgaaaacag gacggacctc catgcctttg agaacctaga aatcatacgc ggcaggacca |
| 3541 | agcaacatgg tcagttttct cttgcagtcg tcagcctgaa cataacatcc ttgggattac |
| 3601 | gctccctcaa ggagataagt gatggagatg tgataatttc aggaaacaaa aatttgtgct |
| 3661 | atgcaaatac aataaactgg aaaaaactgt ttgggacctc cggtcagaaa accaaaatta |
| 3721 | taagcaacag aggtgaaaac agctgcaagg ccacaggcca ggtctgccat gccttgtgct |
| 3781 | cccccgaggg ctgctggggc ccggagccca gggactgcgt ctcttgccgg aatgtcagcc |
| 3841 | gaggcaggga atgcgtggac aagtgcaacc ttctggaggg tgagccaagg gagtttgtgg |
| 3901 | agaactctga gtgcatacag tgccacccag agtgcctgcc tcaggccatg aacatcacct |
| 3961 | gcacaggacg gggaccagac aactgtatcc agtgtgccca ctacattgac ggcccccact |
| 4021 | gcgtcaagac ctgcccggca ggagtcatgg gagaaaacaa caccctggtc tggaagtacg |
| 4081 | cagacgccgg ccatgtgtgc cacctgtgcc atccaaactg cacctacgga tgcactgggc |
| 4141 | caggtcttga aggctgtcca acgaatgggc ctaagatccc gtccatcgcc actgggatgg |
| 4201 | tgggggccct cctcttgctg ctggtggtgg ccctggggat cggcctcttc atgtgagcgg |
| 4261 | ccgctctaga tggccagatc tagcttgtgg aaggctactc gaaatgtttg acccaagtta |
| 4321 | aacaatttaa aggcaatgct accaaatact aattgagtgt atgtaaactt ctgacccact |
| 4381 | gggaatgtga tgaaagaaat aaaagctgaa atgaatcatt ctctctacta ttattctgat |
| 4441 | atttcacatt cttaaaataa agtggtgatc ctaactgacc taagacaggg aatttttact |
| 4501 | aggattaaat gtcaggaatt gtgaaaaagt gagtttaaat gtatttggct aaggtgtatg |
| 4561 | taaacttccg acttcaactg tatagggatc ctctagctag agtcgactgc a |

### Structure of the SLAMF7 CAR Polypeptide

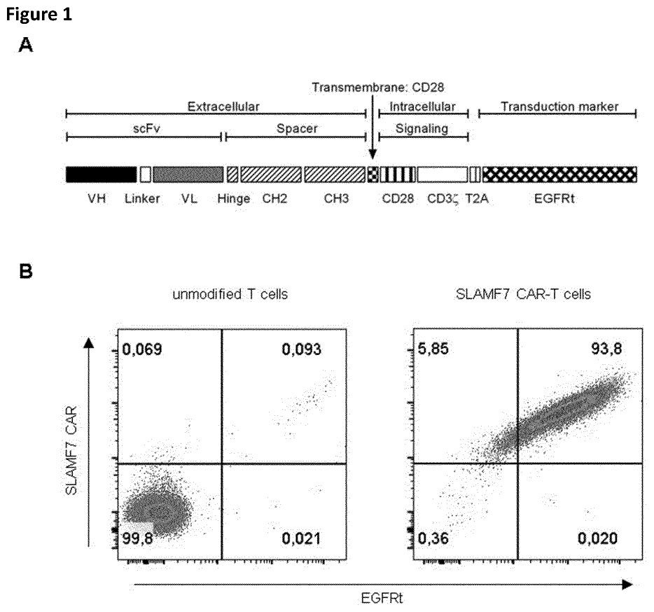

A schematic representation of the gene cassette as expected to be contained in the SLAMF7 CAR T-cell is shown in FIG. 1.

The gene cassette comprising a nucleotide sequence encoding a SLAMF7 CAR polypeptide also contains a truncated epidermal growth factor receptor (EGFRt) sequence, separated from the CAR sequence by a T2A ribosomal skip element to ensure translation of CAR and EGFRt into two separate proteins and stochiometric expression of both proteins on the T cell surface.

The EGFRt protein enables detection and selection of CAR-positive T cells using the anti-EGFR monoclonal antibody cetuximab (trade name: Erbitux®) [26]. In addition, EGFRt opens the option for selective depletion of transgenic T cells with cetuximab in the event of unmanageable toxicity. It was demonstrated in pre-clinical models that administration of cetuximab leads to depletion of CAR-T cells that express EGFRt within few days in vivo [27].

TABLE 2
Annotated sequence of the gene cassette.

| | |
|---|---|
| Signal peptide | MLLLVTSLLL CELPHPAFLL IP (SEQ ID NO: 21) |
| huLuc63 VH | EVQLVESG GGLVQPGGSL RLSCAASGFD FSRYWMSWVR QAPGKGLEWI |
| | GEINPDSSTI NYAPSLKDKF IISRDNAKNS LYLQMNSLRA EDTAVYYCAR |
| | PDGNYWYFDV WGQGTLVTVS S (SEQ ID NO: 17) |
| linker | GGGGSGGGG SGGGGS (SEQ ID NO: 22) |
| huLuc63 VL | DIQM TQSPSSLSAS VGDRVTITCK ASQDVGIAVA WYQQKPGKVP |
| | KLLIYWASTR HTGVPDRFSG SGSGTDFTLT ISSLQPEDVA TYYCQQYSS |
| | YPYTFGQGTK VEIK (SEQ ID NO: 19) |
| IgG4-Fc spacer ″long″ | ESKYGP PCPPCP APPV AGPSVFLFPP KPKDTLMISRT PEVTCVVVDV |
| (Hinge-CH₂-CH₃ with | SQEDPEVQFN WYVDGVEVHN AKTKPREEQF QSTYRVVSVL TVLHQDWLNG |
| 4/2NQ modification) | KEYKCKVSNK GLPSSIEKTI SKAKGQPREP QVYTLPPSQE EMTKNQVSLT |
| | CLVKGFYPSD IAVEWESNGQ PENNYKTTPP VLDSDGSFFL YSRLTVDKSR |
| | WQEGNVFSCS VMHEALHNHY TQKSLSLSLG K (SEQ ID NO: 2) |
| CD28 transmembrane | MFWVLVVVG GVLACYSLLV TVAFIIFWV (SEQ ID NO: 3) |
| CD28 cytoplasmatic | RSKRSRGGHS DYMNMTPRRP GPTRKHYQPY APPRDFAAYR S |
| | (SEQ ID NO: 4) |
| CD3-zeta | RVKFSRSAD APAYQQGQNQ LYNELNLGRR EEYDVLDKRR GRDPEMGGKP |
| | RRKNPQEGLY NELQKDKMAE AYSEIGMKGE RRRGKGHDGL YQGLSTATKD |
| | TYDALHMQAL PPR (SEQ ID NO: 5) |
| T2A seguence | LEGGGEG RGSLLTCGDV EENPGPRM (SEQ ID NO: 23) |
| Signal peptide | LL LVTSLLLCEL PHPAFLLIP (SEQ ID NO: 24) |
| EGFRt | R KVCNGIGIGE FKDSLSINAT NIKHFKNCTS ISGDLHILPV AFRGDSFTHT |
| | PPLDPQELDI LKTVKEITGF LLIQAWPENR TDLHAFENLE IIRGRTKQHG |
| | QFSLAVVSLN ITSLGLRSLK EISDGDVIIS GNKNLCYANT INWKKLFGTS |
| | GQKTKIISNR GENSCKATGQ VCHALCSPEG CWGPEPRDCV SCRNVSRGRE |
| | CVDKCNLLEG EPREFVENSE CIQCHPECLP QAMNITCTGR GPDNCIQCAH |
| | YIDGPHCVKT CPAGVMGENN TLVWKYADAG HVCHLCHPNC TYGCTGPGLE |
| | GCPTNGPKIP SIATGMVGAL LLLLVVALGI GLFM (SEQ ID NO: 15) |

mRNA Coding for SB Transposase
Description of SB mRNA Manufacturing

mRNA encoding the SB transposase can be prepared by the skilled person based on standard protocols and standard materials known in the art as described e.g. in [33], [34] or [35].

The DNA that serves as template for the manufacturing of the SB mRNA is provided as high quality plasmid DNA in endotoxin free water.

The following elements are contained:
T7 promoter: promoter for T7 RNA polymerase
SB100X: Sleeping Beauty transposase gene
polyA: polyadenylation for higher mRNA stability and improved translation ability
AmpR promoter: promoter element for ampicillin resistance gene
AmpR: ampicillin resistance gene
SV40 promoter: promoter element for NeoR/KanR gene Promoter für Resistenzen
NeoR/KanR: neomycin/kanamycin resistance gene
Ori and f1 Ori: origin of replication

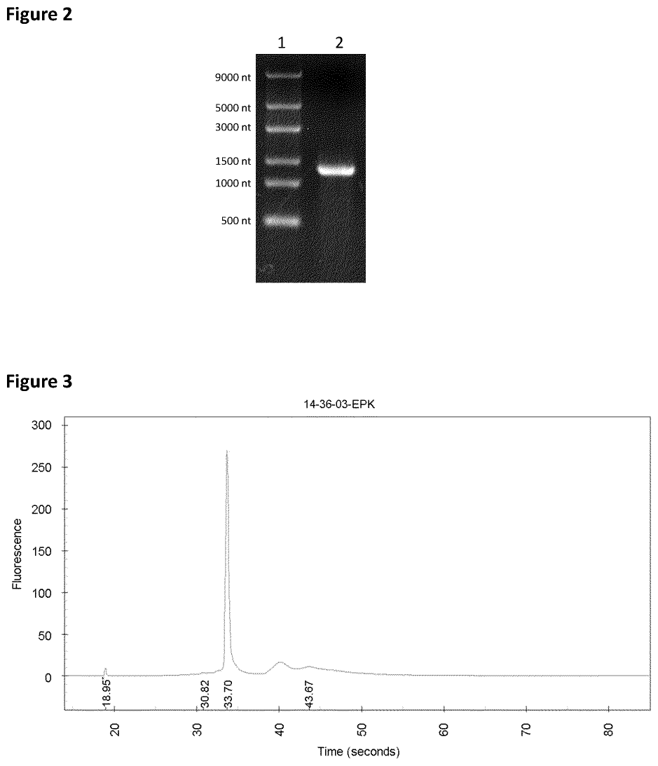

The pcGlobin2-SB100X plasmid is 6637 bp long. The nucleotide sequence of the plasmid is shown in SEQ ID NO: 14. The manufactured SB mRNA is of high purity with an expected length of approximately 1300 nt. FIG. 2 shows a single band of SB100X mRNA (in lane 2) running between the 1000 nt and 1500 nt marker bands (FlashGel RNA Marker Lonza, lane 1), which is in agreement with the expected length of app. 1300 nt.

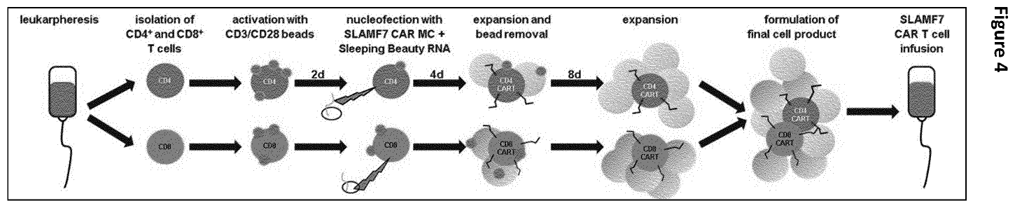

The SB mRNA is of high purity as demonstrated by the electropherogram shown in FIG. 3. The intact RNA results in a distinct peak (retention time of 33.7 s). Degraded or shorter RNA is not detectable in significant amounts. Additional minor peaks at later time point (app. 40 s) are detectable, which presumably represent secondary or tertiary structures of the RNA. Note that the leftmost peak with a retention time of <20 s corresponds to an internal standard added to all samples.

For functional characterization, SB100X RNA (and as control three other RNAs of different length) were translated in vitro using a Rabbit Reticulocyte Lysate Translation System and ³⁵S-Methionine. The labelled translation products were separated by SDS-PAGE and exposed to a phosphor image screen. The protein bands were then analysed on a Phospho-Imager. The assay could verify that SB100X mRNA is translated in vitro into a single protein of the expected size range.

### Manufacture of the Drug Substance (DS) and Drug Product (DP)

### Manufacturing Flow Chart

An artificial separation has been introduced between drug substance and drug product, as the manufacturing process is continuous. In a continuous manufacturing process both CD4⁺ and CD8⁺ T cells are simultaneously but separately undergoing the process steps to yield CD4⁺ SLAMF7 CAR-T cells and CD8⁺ SLAMF7 CAR-T cells. Drug substance is defined as the cells resulting from the harvest step (step 8, see below).

The final cell product is then created by formulating equal proportions of CD8⁺ cytotoxic and CD4⁺ helper SLAMF7 CAR-T cells.

### Process Description

The SLAMF7 CAR-T cell manufacturing process essentially consists of the following step (see also FIG. 4):
isolation of CD8⁺ and CD4⁺ T cells from patient blood and stimulation with anti-CD3/anti-CD28 dynabeads and Interleukin-2
gene modification of T cells to express the SLAMF7 CAR using SB100X transposase RNA and SLAMF7 CAR minicircle DNA
expansion of CD8⁺ and CD4⁺ T cells in separate cultures
formulation of the SLAMF7 CAR-T cell product at a defined CD4:CD8 ratio (1:1 ratio, range 0.5:1 to 2:1)

### Patient Leukapheresis

Leukapheresis of patient blood is performed at ambient temperature at the collection sites, with subsequent controlled shipping of material at 2° C. to 8° C. Apheresis collection sites have to be certified according to EU Directive 2004/23/EC (Setting standards of quality and safety for the donation, procurement, testing, processing, preservation, storage and distribution of human tissues and cells; March 2004) and Commission Directive 2006/17/EC (certain technical requirements for the donation, procurement and testing of human tissues and cells; February 2006) and must have a permit by the local authorities to perform such collections, such as e.g. for German centres a manufacturing license according to German Drug Law § 13. Initially, leukapheresis will only be done at the DRK-BSD (which is also the drug substance and drug product manufacturer).

Testing of blood samples will be performed twice at the clinical sites:
At the screening visit (“screening sample”) to check a patient's eligibility for the clinical trial, and also to have the results of the serologies available, when shipped to the manufacturing facility;
At the time of leukapheresis (“donation sample”) for starting material qualification. For aphereses done at DRK-BSD, the companion blood samples from the day of apheresis will be directly measured. For aphereses done at external sites, testing of companion blood samples from the day of apheresis will be done at the DRK-BSD upon receipt of the apheresis.

The following minimum analyses are performed on the screening and on the donation sample:
HIV 1/2: Anti-HIV1 and anti-HIV2 antibodies, antigen (Ag) p24; Nucleic Acid Test (NAT);
Hepatitis B: HBs antigen, anti-Hepatitis B core antigen (anti-HBc) antibodies, and if positive: anti-Hepatitis B surface antigen (anti-HBs) antibodies; NAT
Hepatitis C: Anti-Hepatitis C virus (anti-HCV) antibodies; NAT
Syphilis: Treponema pallidum Hemagglutination (TPHA) test, if positive then clarified with Treponema pallidum antibody absorption (FTA-ABS) test

All tests are performed (and if necessary updated and amended) according to the local regulations as well as EU Directive 2006/17/EC, and for the DRK-BSD site in particular the current version of German guidelines for hemotherapy.

The leukapheresis is performed and documented according to local SOP procedure at DRK-BSD, or at the leukapheresis centres at the respective clinical trial sites.

Evaluations for the leukapheresis comprise
Cell counting
Volume of apheresate
Donor safety screening (see above)
Any applicable additional local requirements

The process volume (blood volume processed through the apheresis device) will be calculated to target a yield of 4×10′ leukocytes. This cell number has been shown to be appropriate for isolating a sufficient number of CD4⁺ and CD8⁺ T cells and subsequently, to generate the target amount of SLAMF7 CAR-T cells (including the highest dose group).

Upon completion of the leukapheresis procedure, the leukapheresate will be transferred (at 2 to 8° C. in a temperature controlled container) directly to the manufacturing facilities for processing. The leukapheresate may be stored at 2° C. to 8° C. for up to 24 hours between end of leukapheresis and start of further processing.

### Step 1: Receipt of Starting Material

Further processing has to be started within 24 h after end of leukapheresis.

Of note, all of the manufacturing activities described below are performed aseptically under laminar air flow (class B, or class A in B for all open manipulations).

### Step 2: Volume Reduction Step

The apheresate is removed from the collection bag, mixed with selection buffer (CliniMACS PBS/EDTA buffer with 0.5% human serum albumin), centrifuged (10 min, 4° C.), the cells resuspended in selection buffer at 2×10⁸ cells/mL, and are combined in a 50 mL tube over a 40 μm cell strainer. Then the cell suspension is split: 2×10⁹ cells are used for further processing of CD8⁺ cells, and 1.5×10⁹ cells are used for CD4⁺ cells processing. In case these cell numbers are not achieved, 40% of volume are taken for CD4⁺ and 60% for CD8⁺ further processing. Both cell suspensions (intended for CD4⁺ and CD8⁺ selection) are washed with selection buffer followed by centrifugation.

Cell count will be assessed before start of manufacturing using a QC sample taken either directly after apheresis, if performed at DRK-BSD, or a separate QC sample will be taken after delivery to DRK-BSD (sample also used for serology). Sampling for sterility testing will be performed using the volume left in apheresis bag in the GMP facility.

Step 3: Immunomagnetic Separation of CD4⁺ and CD8⁺ Cells from PBMC

The cell pellet as obtained in step 2 is resuspended in selection buffer (volume dependent on the cell number, at a ratio of 300 μL buffer per 10⁸ cells).

Gamunex 10% (containing 10% w/v immunoglobulin G) is added at a ratio of 100 μL per 10⁸ cells.

Then CD4 CliniMACS reagent is added to the CD4⁺ cell suspension, and CD8 CliniMACS reagent is added to the CD8⁺ cells. After incubation for 30 min at 2° C.-8° C., the cells are washed in selection buffer, centrifuged and resuspended in cold selection buffer (at a ratio of 1 mL buffer per 10⁸ cells).

An appropriate number of LS columns (1 column for up to 5×10⁸ cells) is equilibrated in selection buffer, and put in the magnetic field of the QuadroMACS separator. The cell suspensions are loaded onto the LS columns, and the columns are washed 3 times with selection buffer. Afterwards the columns are removed from the magnet separator and the cells are eluted by adding 10 mL selection buffer.

An aliquot is taken for determination of cell count and viability.

Cell suspensions are centrifuged, resuspended in pre-warmed complemented cell culture medium (DMEM with 10% human plasma, 1% GlutaMax supplement, complemented with 50 IU/mL IL-2) and seeded in T75 cell culture flasks at 75×10⁶ cells/20 mL/flask. 150×10⁶ of both purified CD4⁺ and CD8⁺ cells, respectively, are used for further processing. Any remaining cells are stored away as retain samples.

From each T cell subset at least 3×10⁵ cells are stained with anti-CD38 and anti-CD138 antibodies and 7AAD to detect living residual MM cells by flow cytometry.

Step 4: In Vitro Stimulation with CD3/CD28 Dynabeads

CTS Dynabeads CD3/CD28 stock solution is mixed with the same amount of complemented cell culture medium and an amount of one dynabead per cell is added to the T75 flasks with the CD4⁺ and CD8⁺ T cells.

In case less than 150×10⁶ cells are available from step 3, these are processed further as well according to the described procedure. A minimal cell number of 100×10⁶ cells per population needs to be achieved.

Afterwards, a sample from both the CD4⁺ and CD8⁺ T cell flasks is taken and put on sterility testing (BacT/ALERT®.). Cells are maintained in culture at 5% CO₂ and 37° C. for 48 h.

### Step 5: Nucleofection

The gene transfer of the SLAMF7 construct into the T cells (both CD4⁺ and CD8⁺) is performed by electroporation with the Lonza nucleofector device, using a non-viral vector (Sleeping Beauty transposase mRNA which upon nucleofection is transcribed within the cells into the SB transposase) and a minicircle DNA carrying the SLAMF7 CAR construct.

The purified and activated T cells in the T75 flasks are harvested, centrifuged and suspended in sterile PBS. An IPC sample is taken, and viable cell count determined by trypan blue measurement. Sterile PBS is added to the cell suspension to reach 10⁷ cells/mL.

Separate nucleofections are done with the CD4⁺ and CD8⁺ cells, using 4×10⁷ CD4⁺ cells and 6×10⁷ CD8⁺ cells, respectively. The remaining T cells—if any—are seeded in culture medium in T25 flasks as control cultures (to provide a baseline control for non-transfected cells in later flow cytometry measurements).

Nucleofection is prepared by centrifuging cells, resuspension in nucleofection solution and supplement, and adding minicircle DNA (5 μg/10⁷ cells) and SB transposase mRNA (20 μg/10⁷ cells). Nucleofection is performed in dedicated sterile cuvettes in the Lonza nucleofector device, using the program “T cells unst.HF, Pulse Code EO 115”.

Afterwards pre-warmed complemented cell culture medium is added and the cells are incubated at 5% CO₂ and 37° C. in 12 well-plates. After 4 h, the supernatant is removed and freshly complemented cell culture medium (containing IL-2 at 50 IU/mL) is added. Samples of the removed supernatant are subjected to sterility testing.

### Step 6: Cell Expansion

Cell cultures as obtained in step 5 are incubated at 5% CO2 and 37° C. for 3 days. For the nucleofected CD4⁺ and CD8⁺ cell cultures a partial media exchange is done by carefully removing half of the volume of the supernatant and adding the same volume of fresh culture medium (complemented by IL-2 to reach a final concentration of 50 IU/mL). Cells are then gently resuspended. The same is performed for the control culture (not nucleofected) in the T25 flasks.

Afterwards incubation of the cell cultures is continued for one day.

### Step 7: Dynabead Removal and Further Expansion

The nucleofected cells are transferred to 50 mL tubes and dynabead removal is performed by placing the cell suspension for 1 min into the Invitrogen Dynamag 50 magnet system. The beads are forced to the inner surface of the tubes, so the cells can be removed and are then transferred to a fresh 50 mL tube. Dynabeads are washed with PBS and the same procedure is repeated with the resuspended beads, to harvest remaining cells.

The obtained cells are centrifuged and both the CD4⁺ and CD8⁺ cells are (separately) brought into separate 10 mL of complemented cell culture medium. Samples are taken for cell counting and FACS analyses. After addition of the necessary volume of complemented cell culture medium for further processing (see below), samples for sterility testing are taken.

Cell are transferred for further culture into G-Rex10M gas permeable cell culture devices (in the following referred to as G-Rex10M). The number of G-Rex10M is dependent on the number of viable cells: 5-15×10⁶ cells/100 mL cell culture medium/G-Rex10M are used, with a maximum of three G-Rex10M for CD8⁺ cells and two G-Rex10M for CD4⁺ cells. An IPC sample for sterility test is taken.

The same procedure is performed for the non-nucleofected (control) cells. The only difference is that they will be further cultured in T25 flasks (at 10⁷ cells/15 mL).

All cells are incubated at 5% CO2 and 37° C.

After a further 1, 3 and 6 days (days 7, 9, and 12 of the process, respectively) IL-2 is added to the G-Rex10M. For the control cells flasks, a partial exchange of half the volume of medium (supplemented with IL-2 to reach a final concentration of 50 IU/mL) is done. At procedure day 12, a Mycoplasma test is performed from the cell culture supernatant.

### Step 8: Cell Harvest

Cells from step 7 are cultivated for a further 2 days, and at day 14 of the process the cell harvest is done.

60 mL medium supernatant from each G-Rex10M is removed and discarded, the cells are suspended in the remaining medium. Aliquots from the suspensions are taken for determination of total and viable cell count, as well as flow cytometry analyses. In case of a positive test for MM cells on day 0 in either purified T cell population, a second flow cytometric analysis for residual MM cells is performed.

The cells are maintained in culture at 5% CO₂ and 37° C. until the result from the IPC becomes available.

CD4⁺ cells are removed from their G-Rex10M, centrifuged and resuspended in infusion solution (0.45% NaCl, 2.5% glucose, 1% human serum albumin). If more than one G-Rex10M was used (default target would be 2×G-Rex10M per cell population), the cells are combined, then a second centrifugation is done. Again, the cell pellets are resuspended in infusion solution. After a third centrifugation the cells are resuspended at a concentration of 6×10⁶ viable EGFRt⁺ cells/mL infusion solution (the EGFRt is co-expressed in the same amount as the SLAMF7 CAR, so serves as a marker for cells carrying the CAR gene construct). The control culture cells are treated in the same way.

The same procedure is done for the CD8⁺ cells.

Both T cell suspensions are separately examined by microscope for residual dynabeads. If the limit value of 3 beads/200 cells is exceeded, the bead removal step is repeated. For this, T cells are resuspended several times, before the tube is placed for 1 min into the Invitrogen Dynamag 50 magnet system. Due to the magnetic field, the magnetic beads will stick to the wall of the tube. The cell suspension is transferred without the beads into a new tube, and cells are again microscopically examined for residual dynabeads.

The endpoint of this step, i.e. separate CD4⁺ and CD8⁺ cell suspensions, are regarded as drug substance. Without interruption, in a continuous process, they will now be combined to the drug product in step 9.

Steps 9 (Combine CD4⁺ and CD8⁺ CAR T Cells at 1:1 Ratio, Dilute as Required by Patient Dose Group and Body Weight, and Transfer in Infusion Bag) and Step 10 (Labelling).

The manufacturing of the SLAMF7 CAR T cells is an un-interrupted process, with DP manufacturing only constituting the last step of combining the cells and transferring into the infusion bag.

There is a continuous process leading to drug product, with no holding step at the DS level, so DS is immediately processed into DP in the following way:
Calculation of required cells per individual patient, depending on dose group and body weight
Calculation of required volume per individual patient, depending on the body weight
Dilution steps as required with infusion solution
Filling into CryoMACS bags
Labelling of the bags

The individual amount of cells and volume of cell suspension to be administered per patient will vary dependent on dose group and body weight of the particular patient. The necessary cell number is calculated considering the individual patient dose group (1×10⁴ cells/kg for dose level 0, 3×10⁴ cells/kg for sentinel, 1×10⁵ cells/kg for dose level 1, 3×10⁵ cells/kg for dose level 2 or 1×10⁶ EGFRt⁺ T cells/kg for dose level 3, respectively) and considering the particular patient's bodyweight. The necessary volumes carrying same amounts of viable transfected EGFRt⁺ (i.e. CAR-positive) CD4⁺ and CD8⁺ cells are combined, so the cell populations are now combined in a 1:1 fashion.

This is done for the dose groups in the following way:
For the dose group of 1×10⁶ cells/kg: each 5×10⁵ viable CAR-positive CD4⁺ and 5×10⁵ viable CAR-positive CD8⁺ cells, per kg patient body weight
For the dose group of 3×10⁵ cells/kg: each 1.5×10⁵ viable CAR-positive CD4⁺ and 1.5×10⁵ viable CAR-positive CD8⁺ cells, per kg patient body weight
For the dose group of 1×10⁵ cells/kg: each 5×10⁴ viable CAR-positive CD4⁺ and 5×10⁴ viable CAR-positive CD8⁺ cells, per kg patient body weight
For the dose group of 3×10⁴ cells/kg: each 1.5×10⁴ viable CAR-positive CD4⁺ and 1.5×10⁴ viable CAR-positive CD8⁺ cells, per kg patient body weight
For the dose group of 1×10⁴ cells/kg: each 5×10³ viable CAR-positive CD4⁺ and 5×10³ viable CAR-positive CD8⁺ cells, per kg patient body weight

Should in exceptional cases the cell numbers of one of the cell populations not suffice for the planned dose and body weight, then the ratio of CD4⁺ and CD8⁺ cells could be adapted up to 0.5:1 or 2:1, respectively.

Depending on the calculated amount of cells and volume, any necessary prior dilutions will be done by mixing equal amounts of viable transfected EGFRt⁺ (i.e. CAR-positive) CD4⁺ and CD8⁺ DS cells with appropriate volumes of infusion solution (0.45% NaCl, 2.5% glucose, 1% human serum albumin) in transfer bags. Samples for final product release testing are taken, and aliquots with the appropriate cell numbers are transferred from these transfer bags to the CryoMACS bags.

Depending on the body weight and resulting final volumes, the size of the CryoMACS bags for the final product will be selected, according to the set-up as displayed in Table 3.

TABLE 3
Selection of CryoMACs size dependent on fill volume.

| | | | Maximum volume | |
|---|---|---|---|---|
| Patient body weight | | CryoMACS bags | that will be filled | |
| 20-70 | kg | CryoMACS freezing bag 250 | 20-70 | mL |
| | | mL | | |
| 71-100 | kg | CryoMACS freezing bag 500 | 71-100 | mL |
| | | mL | | |
| ≥100 | kg | CryoMACS freezing bag 750 | ≥100 | mL |
| | | mL | | |

The drug product is a cell suspension in a CryoMACS bag in a sterile infusion solution (0.45% NaCl, 2.5% glucose, 1% HSA) at a final volume of 1 mL/kg patient body weight. The cell number is individualized and depends on patient dose group and body weight.

All bags are clearly labelled, and upon QC and OP-release are then transferred to the patient administration site at 2° C.-8° C.

### Properties and Formulation of Drug Product (DP)

### Characteristics of the DP

### T Cell Phenotype

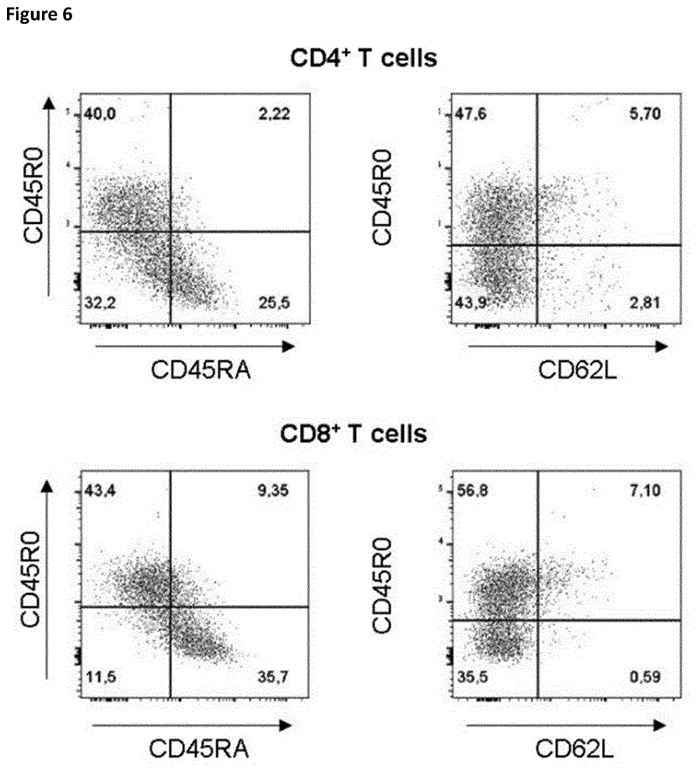

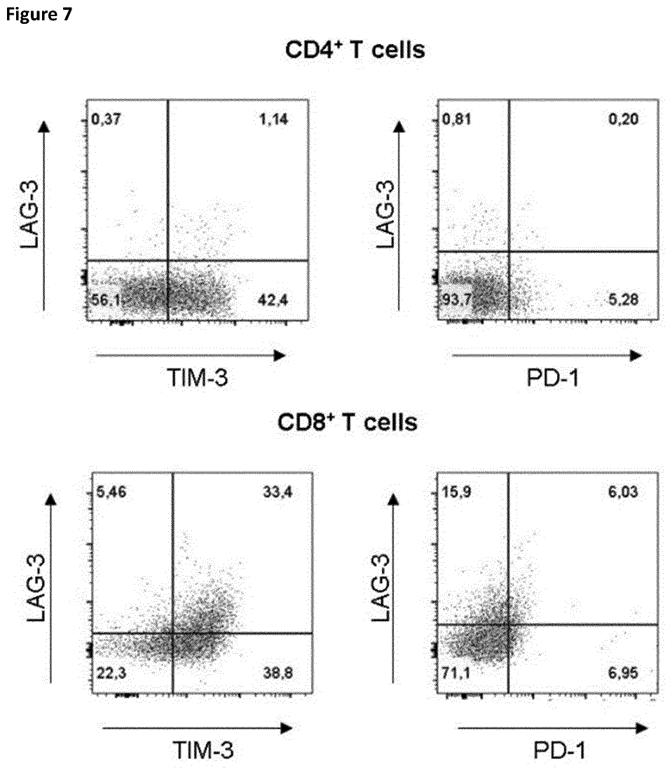

The differentiation state of SLAMF7 CAR-T cells was tested by flow cytometry. The cells of the DP predominantly had an effector cell phenotype, characterized by a CD45RA⁻, CD45RO⁺, CD62L⁻ expression profile (FIG. 6). The DP cells were further tested for expression of activation/exhaustion markers, namely PD-1, TIM-3 and LAG-3. There was no prevalent accumulation of PD-1, TIM-3 and LAG-3 on the surface of SLAMF7 CAR-T cells (FIG. 7). The mean gene transfer rate achieved in n=4 manufacturing runs from healthy donors, was 51.9% and 71.4% in CD4⁺ and CD8⁺ T cells, respectively.

### Vector Copy Number of Genomically Integrated SLAMF7 CAR Transposons

With any vector that integrates into chromosomes in a semi-random manner, comes a theoretical risk of insertional mutagenesis leading to transcriptional activation or inactivation of cellular genes. There are at least two factors that contribute to the potential genotoxicity of an integrating vector system: i) vector copy number per genome and ii) genome-wide integration profile.

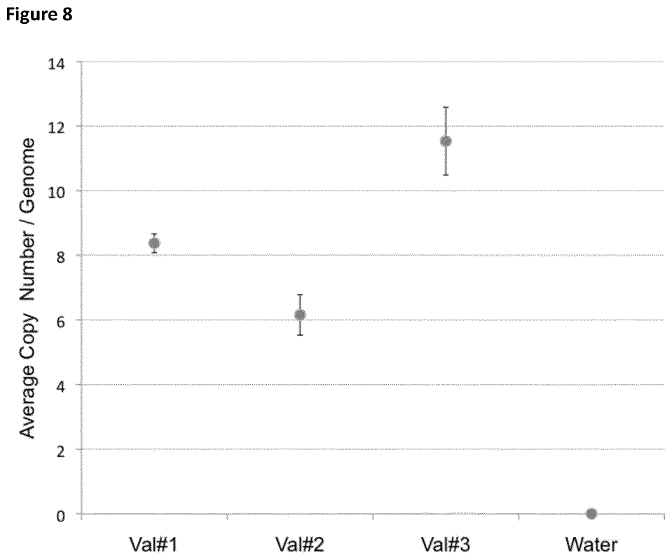

Three validation runs were conducted to produce SLAMF7 CAR-T cell DP. The numbers of SB transposon integrations obtained per T cell genome was determined by quantitative droplet digital polymerase chain reaction using the cells of these three validation run DP. The average vector copy number (VCN) per diploid genome in the three validation runs was 8, 6 and 12 (FIG. 8).

### Insertion Profile of the SLAMF7 CAR Sleeping Beauty Vectors in the T Cell Genome

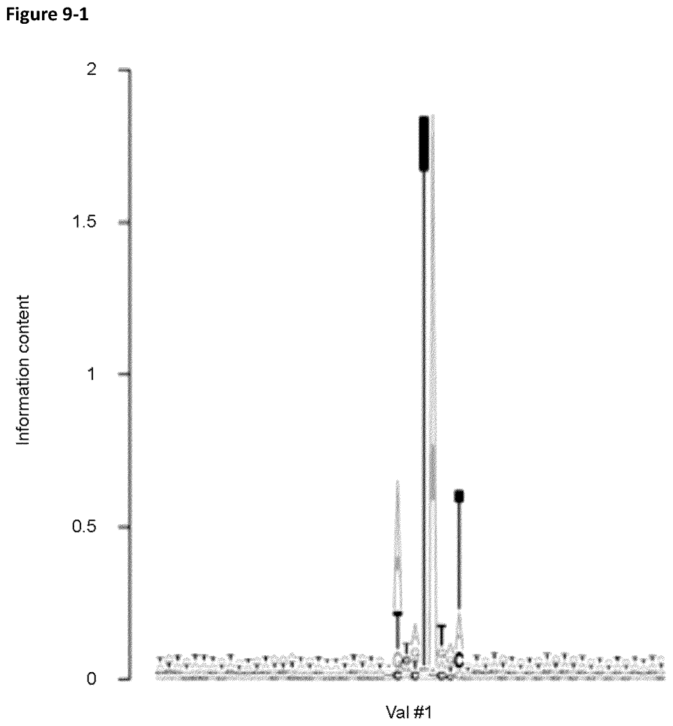

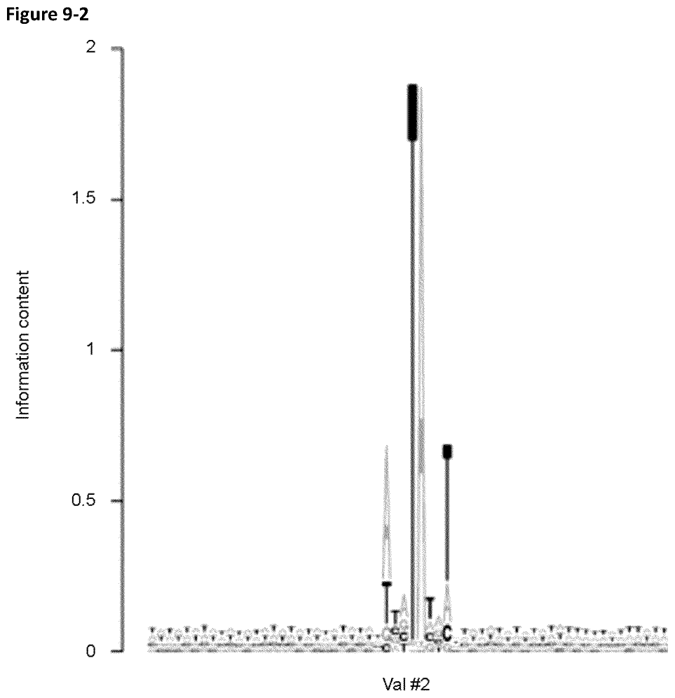

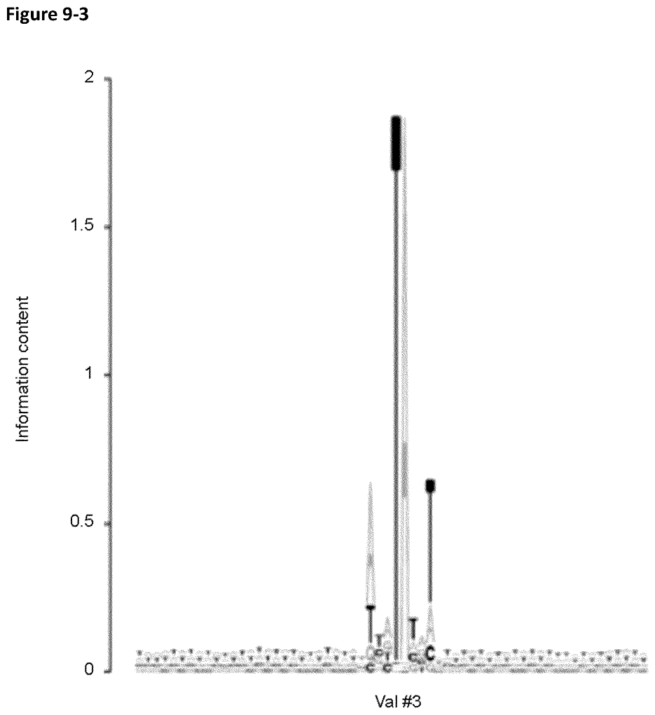

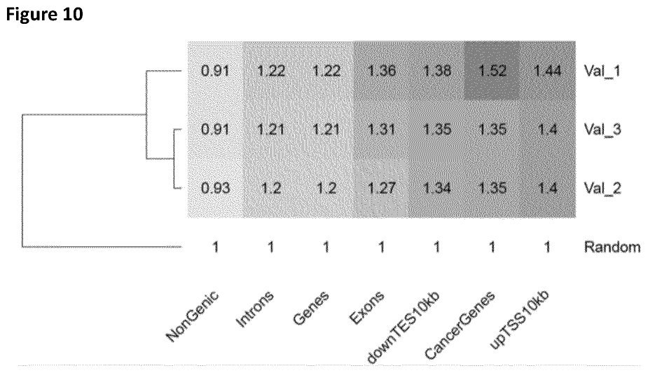

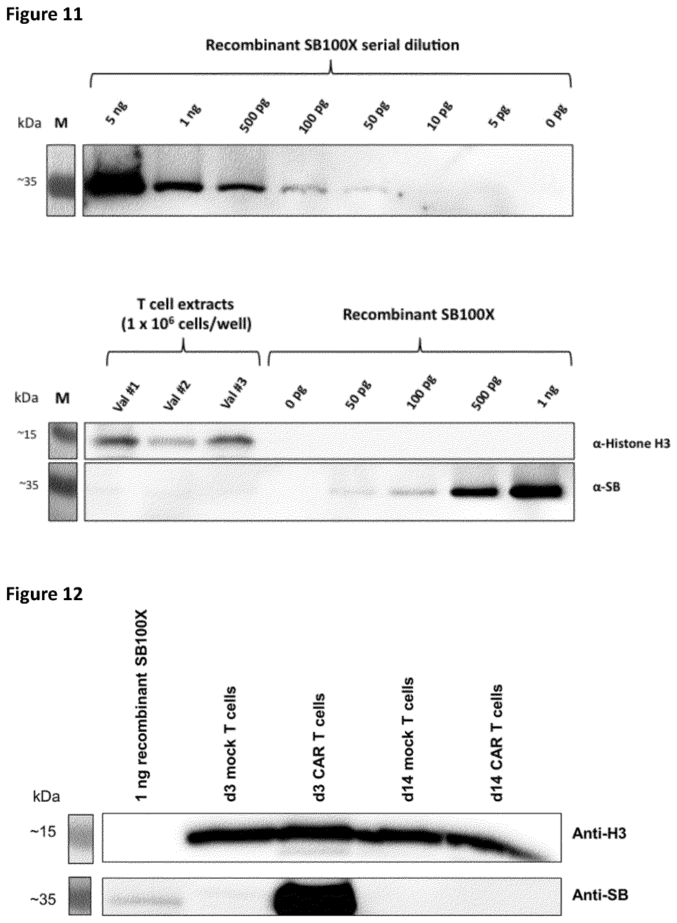

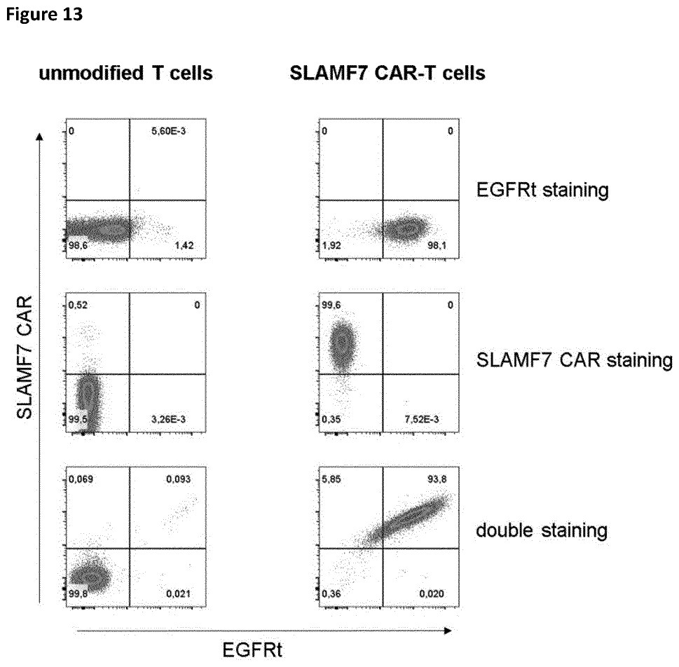

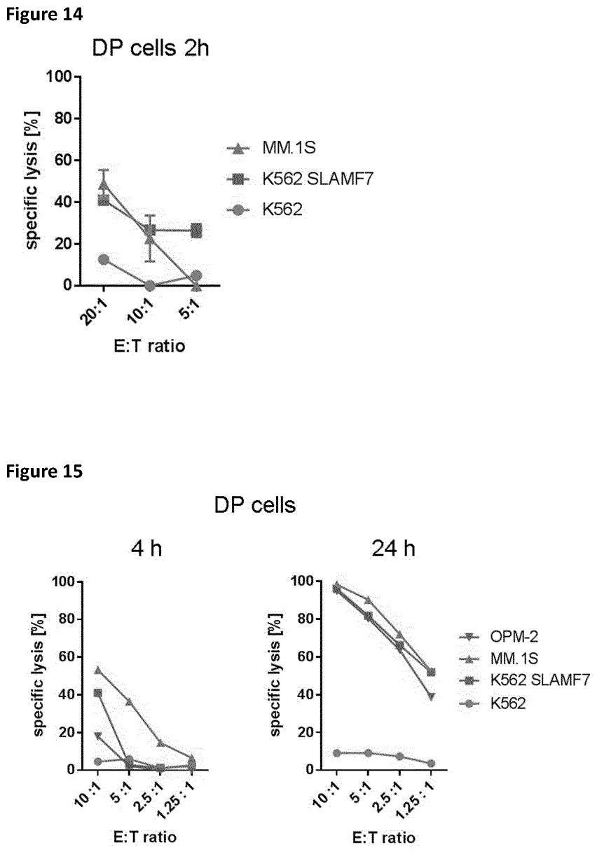

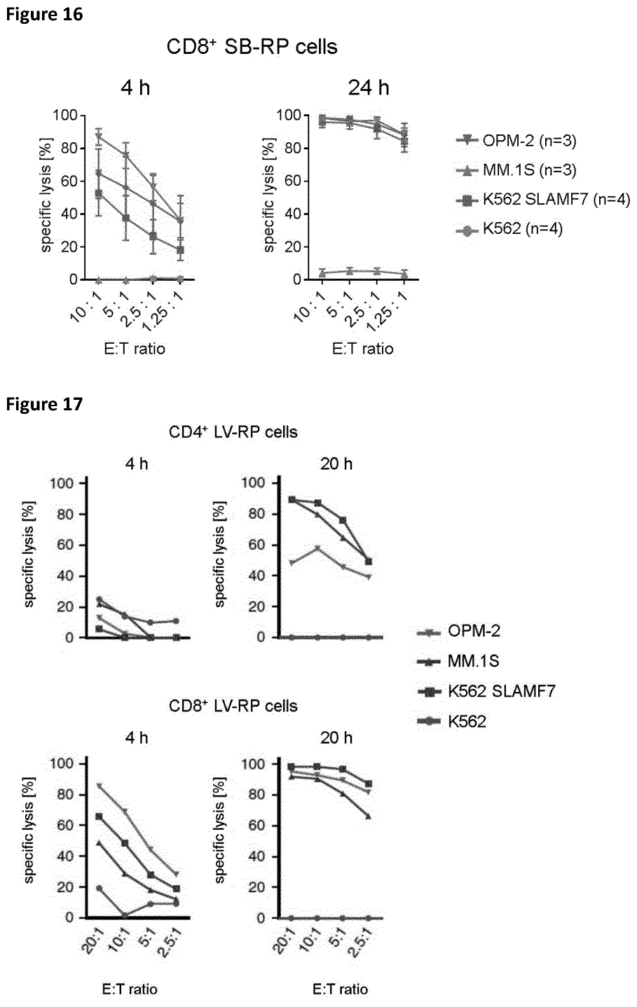

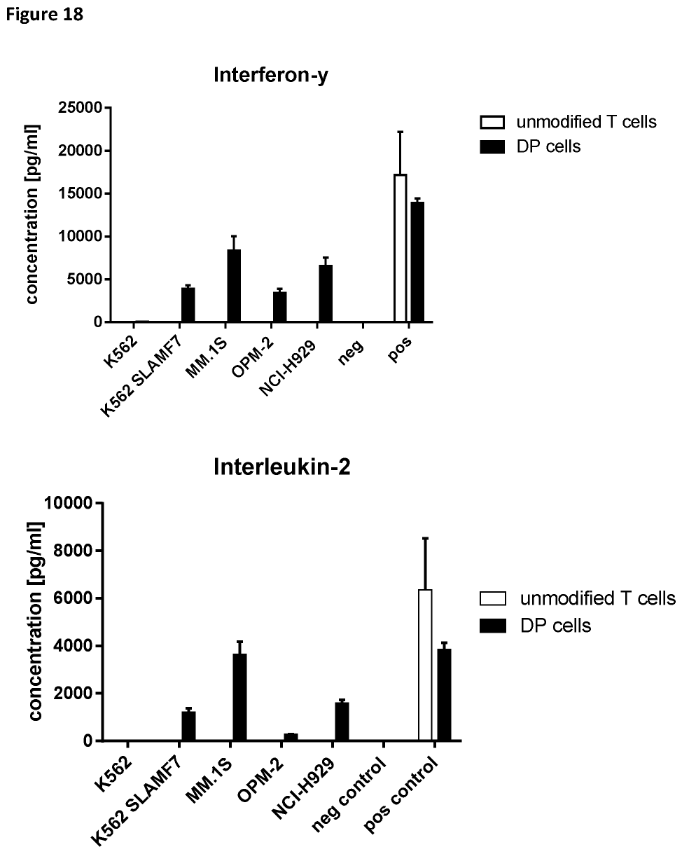

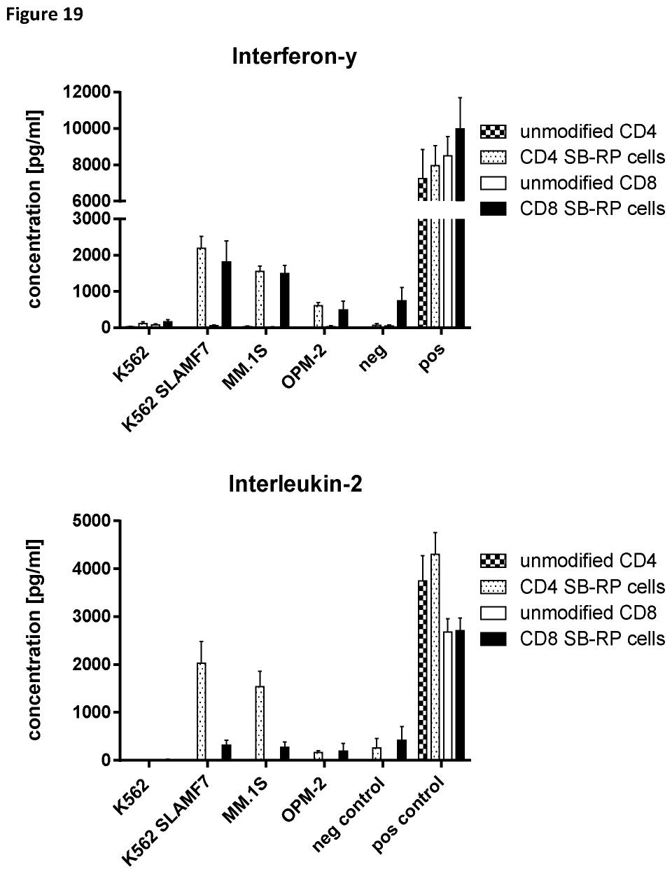

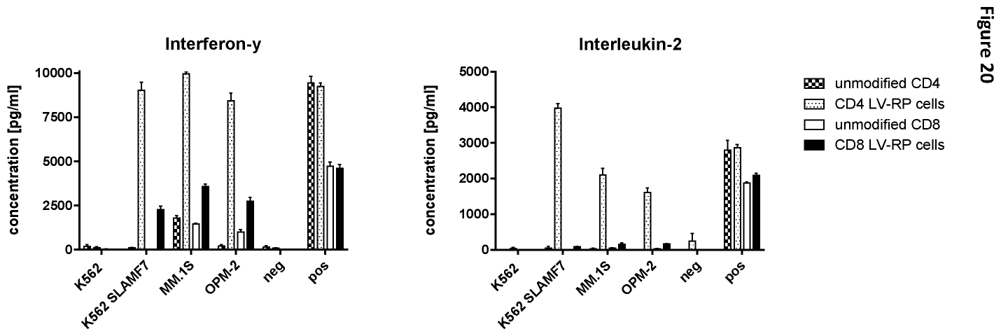

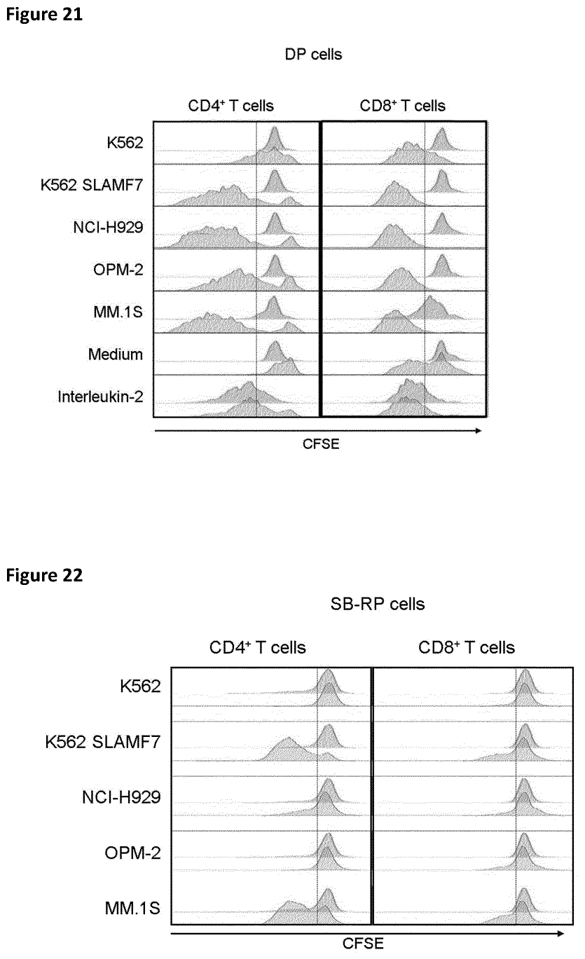

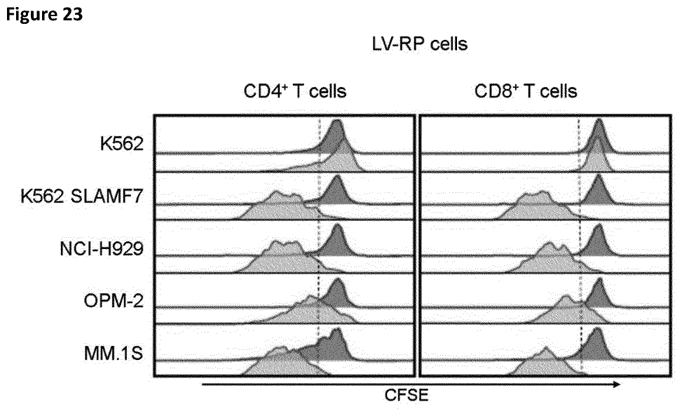

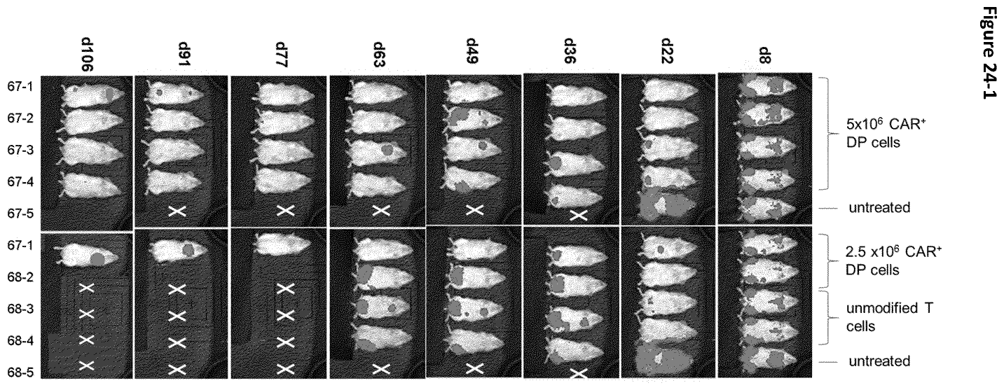

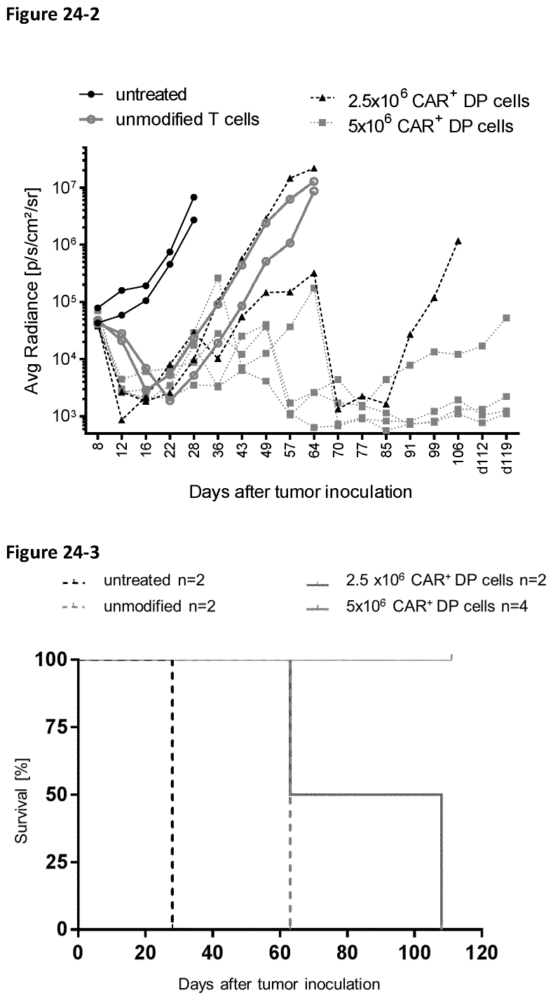

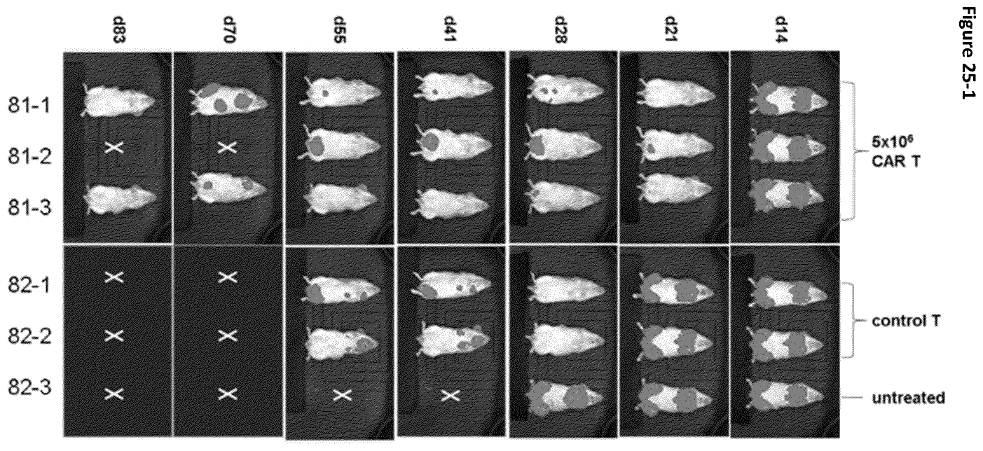

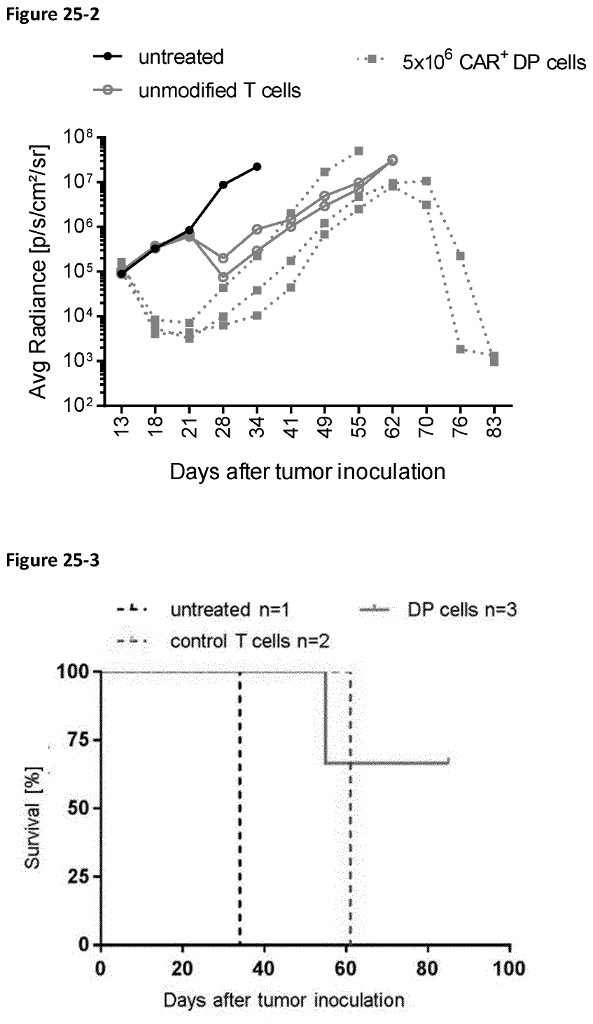

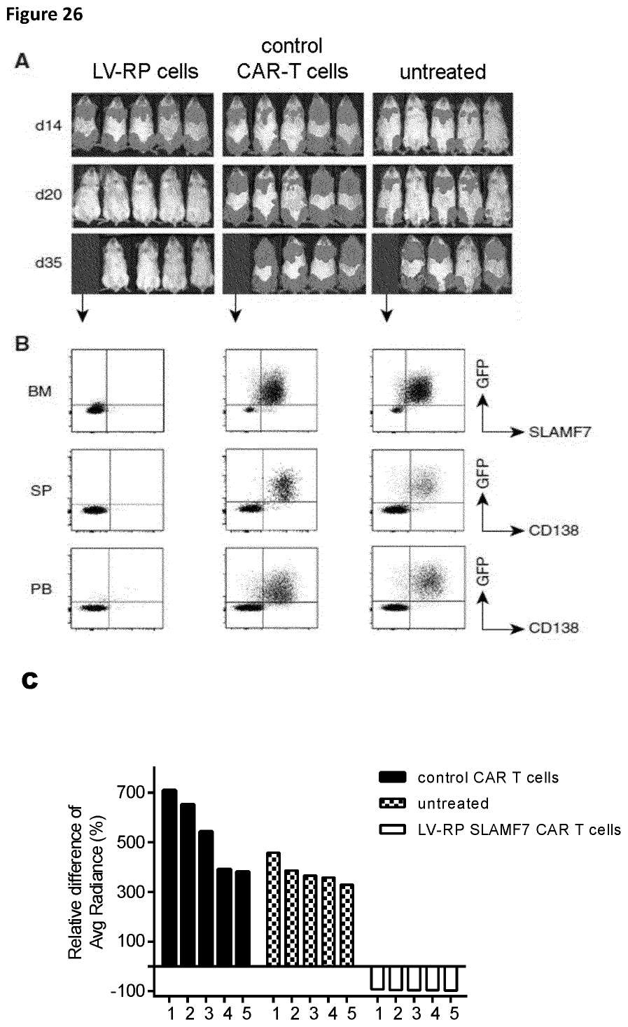

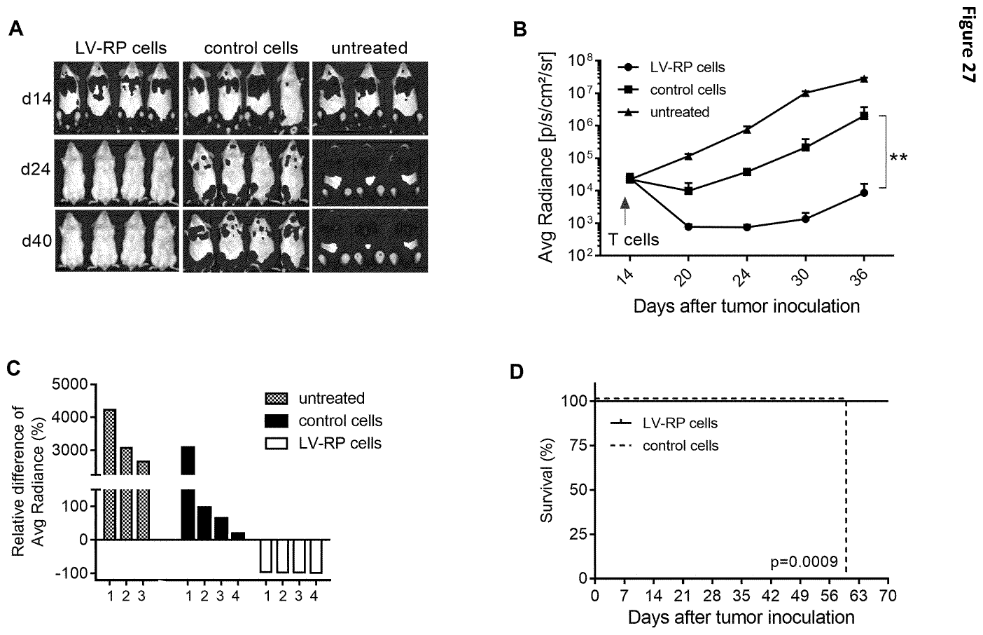

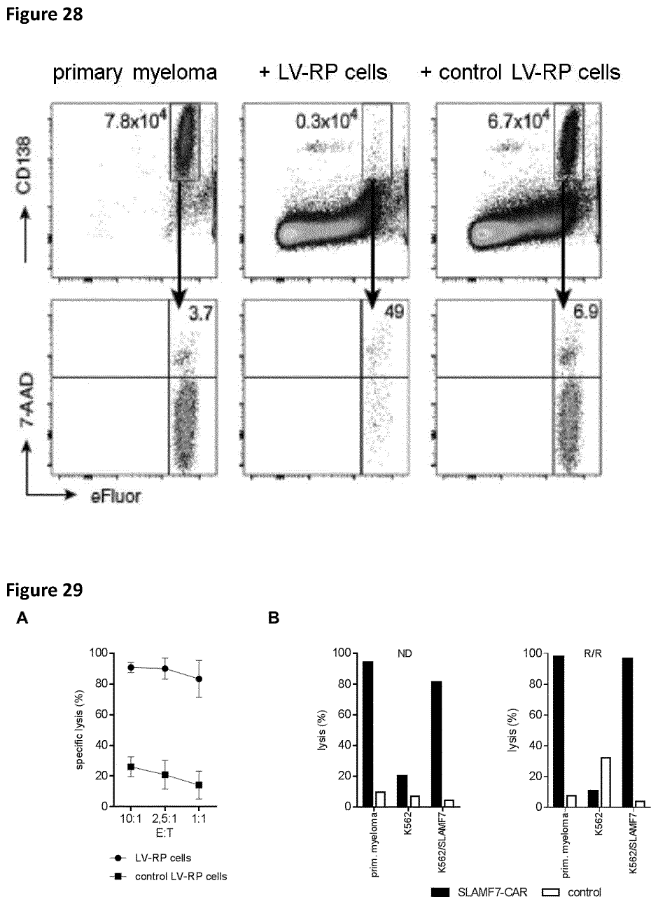

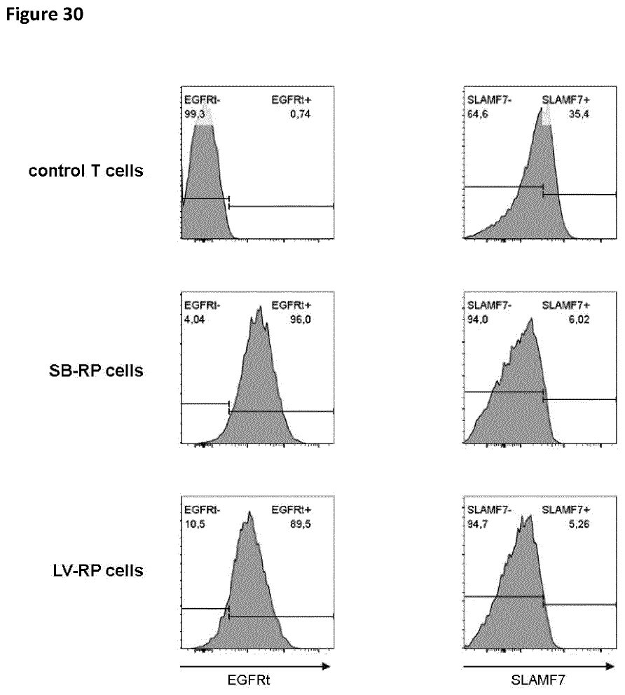

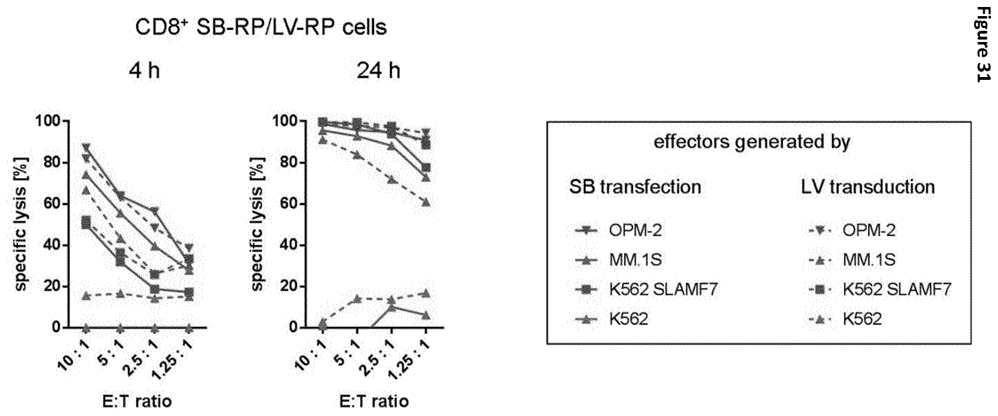

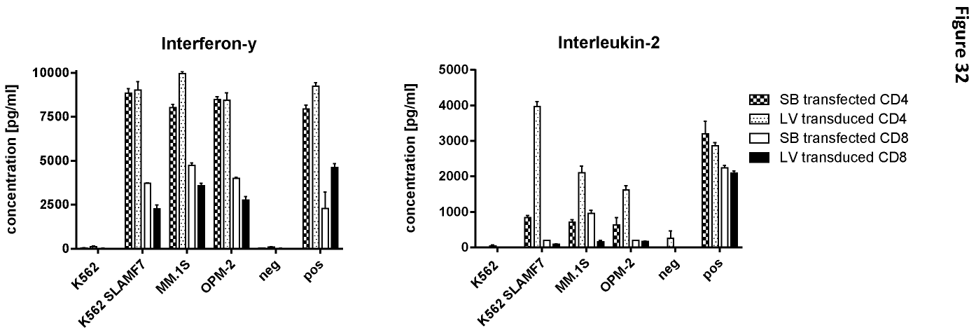

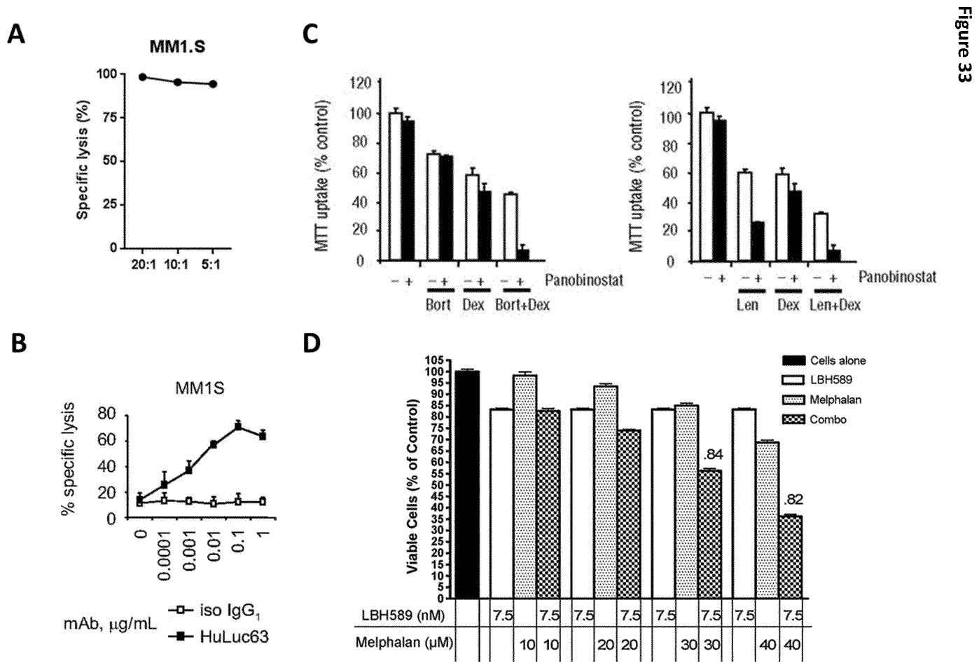

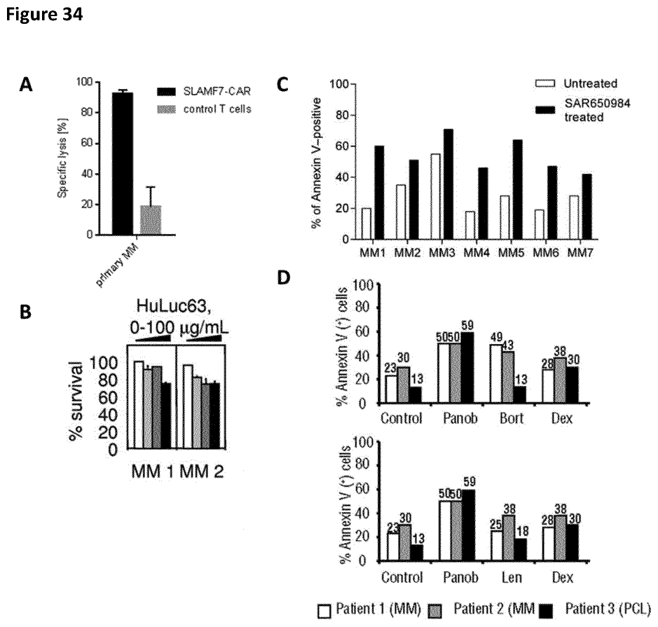

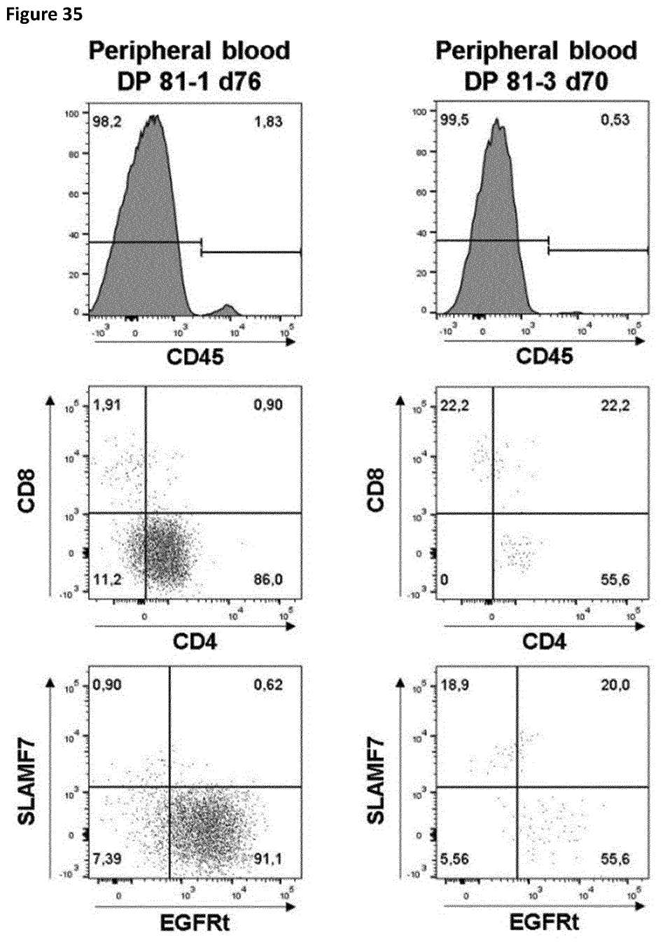

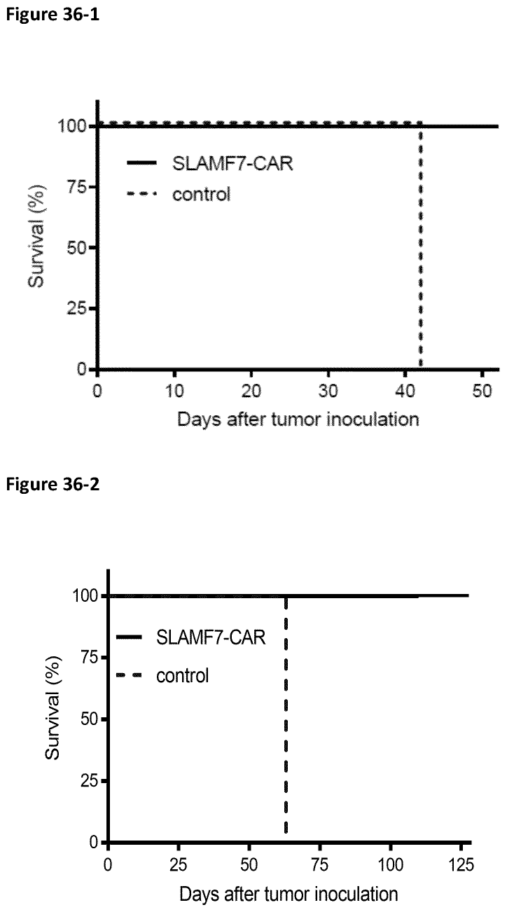

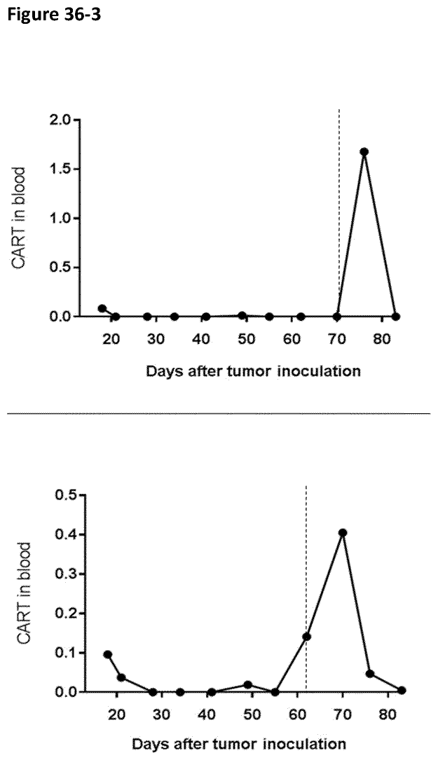

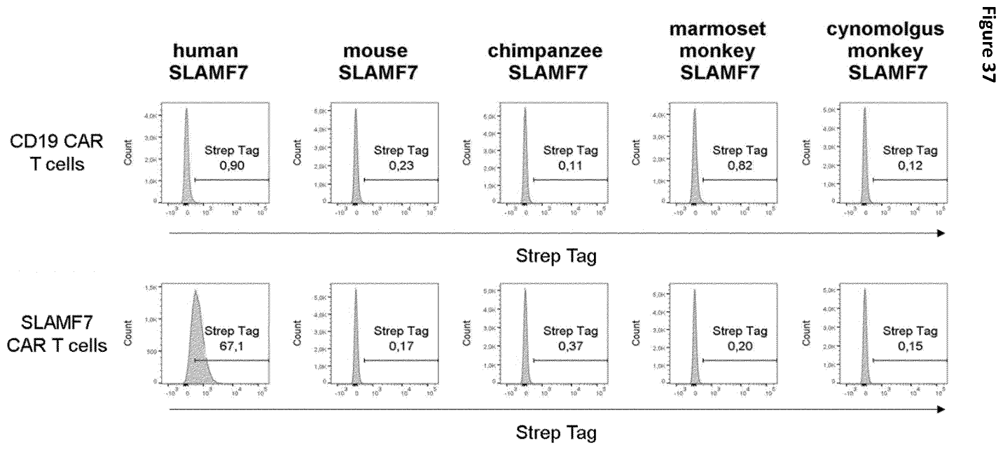

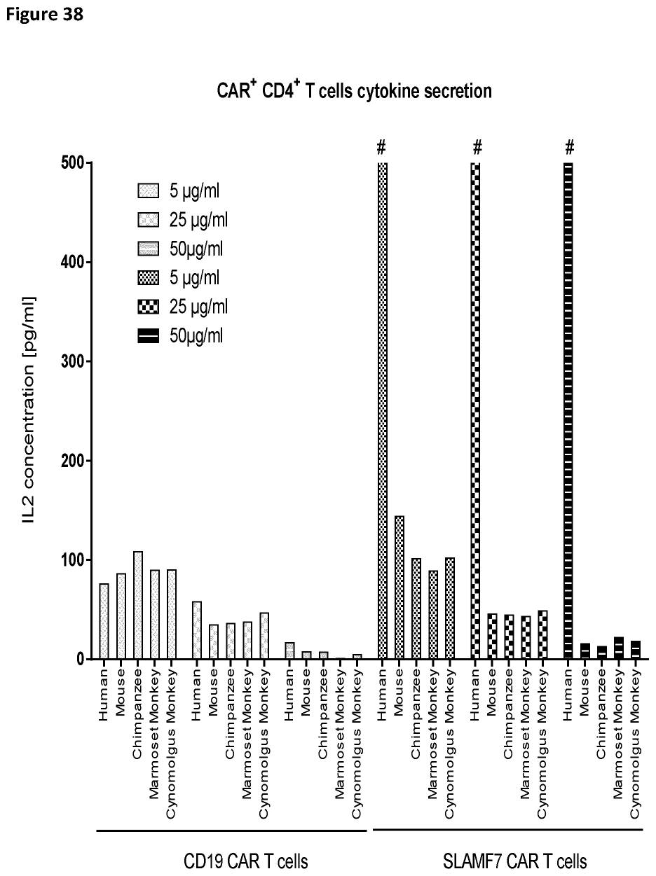

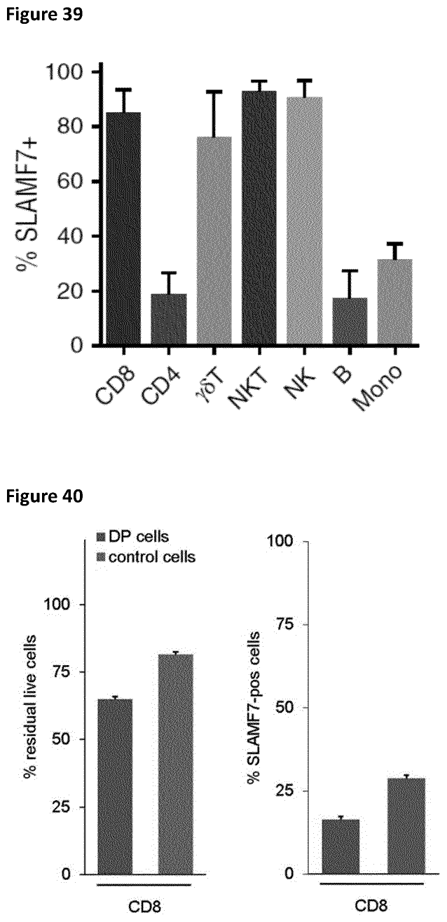

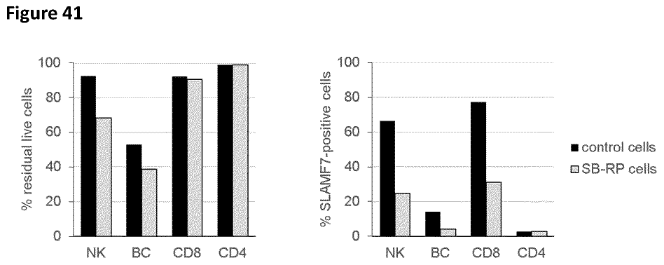

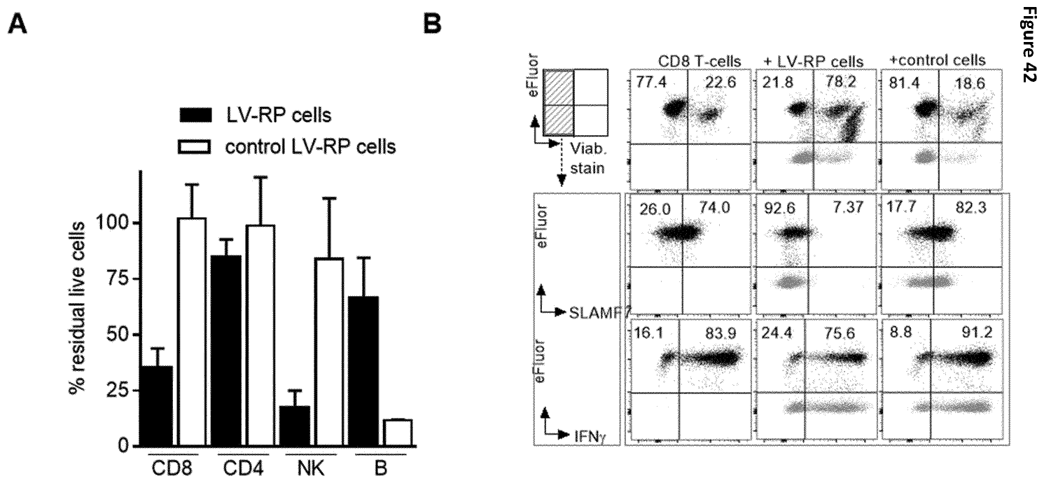

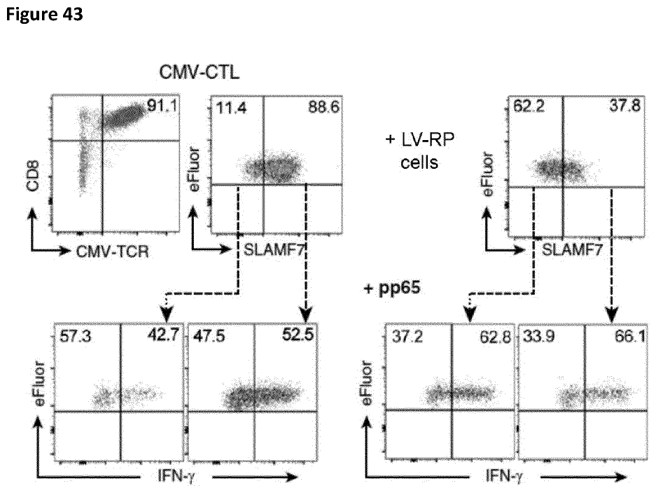

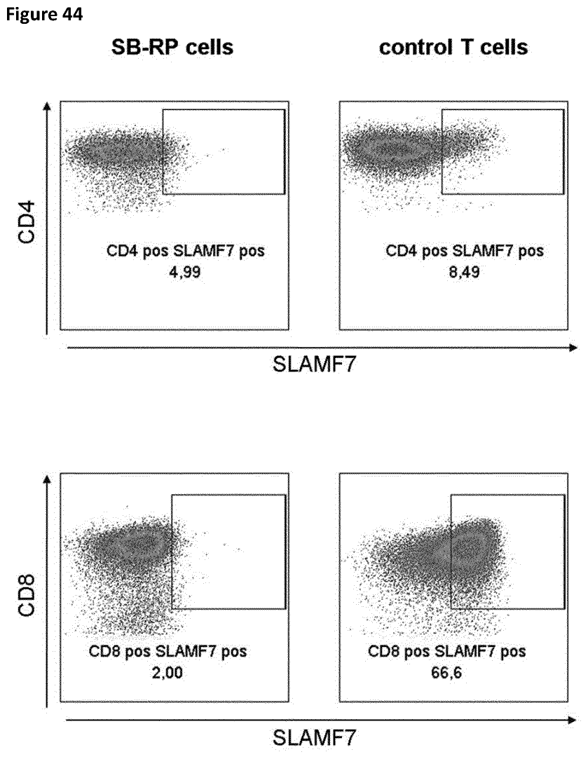

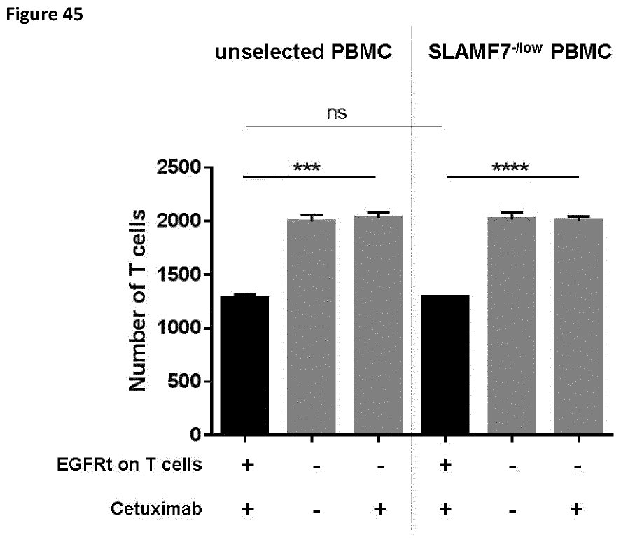

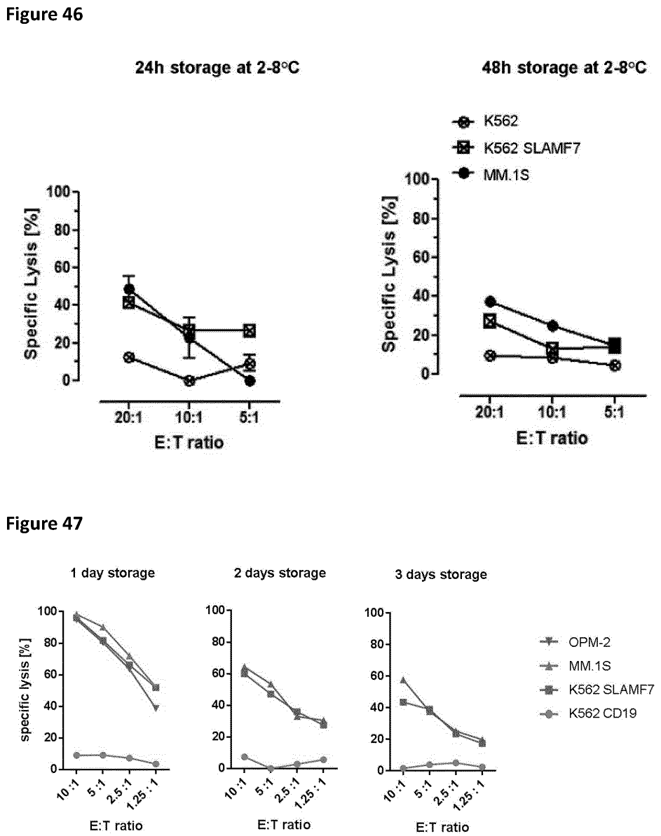

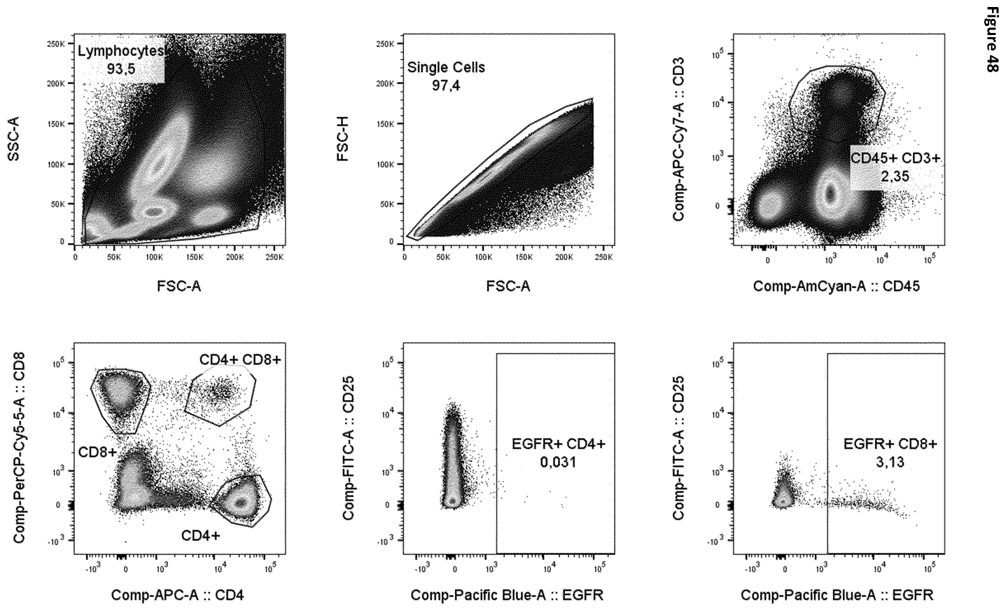

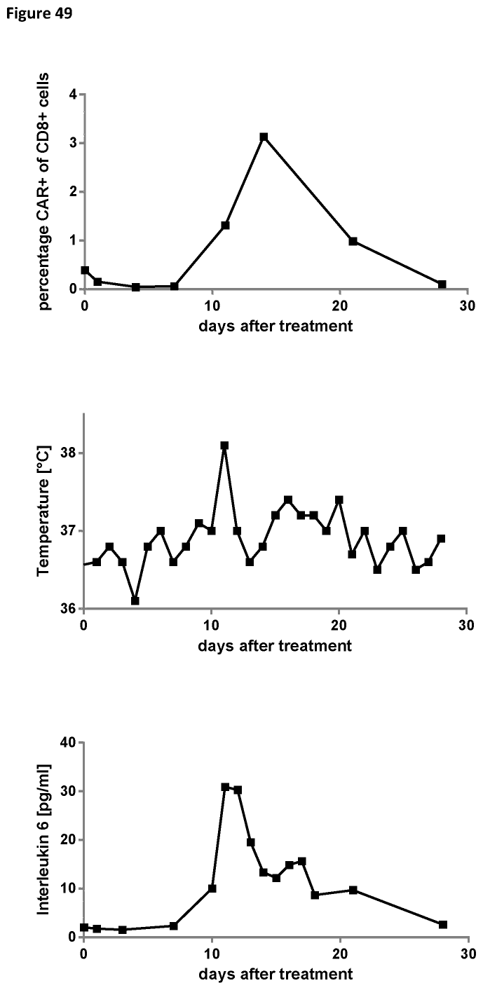

Insertion site libraries from SLAMF7 CAR-T cells (DP produced in 3 validation runs) were constructed for massive parallel sequencing on the Illumina MiSeq platform using standard methods. From these three independent samples 5738, 6349 and 18574 unique insertion sites of the SLAMF7 CAR transposon were mapped and characterized. The characteristic palindromic ATATATAT motif was detected, which contains the TA dinucleotide target sequence of SB adjacent to all MC-derived transposons (FIG. 9).

Further, it was analysed whether there was a preference of SLAMF7 CAR transposon insertions into distinct sites of the genome, e.g. exons and introns, genes and cancer-related genes. Transpositions had occurred with only a modest, yet statistically significant (p<0.001) bias towards genic categories (FIG. 10); however, it has been surprisingly shown that in all evaluated categories this preference was substantially smaller than previously found for lentiviral and gammaretroviral integrations ([28], [29]). Importantly, transposon insertions showed only a ^{˜}1.2-fold enrichment in genes and a ^{˜}1.4-fold enrichment in cancer-related genes relative to the expected random frequency. Concordantly, CAR transposons were also inserted into non-genic regions in a close-to-random manner (^{˜}0.9-fold compared to random; FIG. 10).

Hence, it has been surprisingly found by the present inventors that SB transposition allows a safer integration of a nucleotide sequences encoding the CAR polypeptide of the invention compared to known viral based integration methods.

### Residual Transposase

In order to address the potential presence of the SB100X transposase at the end of the manufacturing period, cell populations from three independent validation runs were collected, and protein extracts analysed by Western blotting alongside known amounts of recombinant purified SB100X transposase (FIG. 11). The detection limit of SB100X through chemiluminescent Western blotting in this procedure was 50 pg, which corresponds to ^{˜}7.66×10⁸ SB100X transposase molecules (SB100X=^{˜}39.29 kDa). Because there was no band corresponding to the SB100X transposase in the SLAMF7 CAR-T cell extracts (FIG. 11), the detection limit of this procedure is ^{˜}766 SB100X molecules per cell, and all three validation runs contained residual SB transposase below this limit. Furthermore, considering the half-life of SB transposase protein and the length of the culture process, the SLAMF7 CAR-T cell product can be considered to be negative for residual transposase.

In contrast to harvest day, SB100X transposase protein is highly detectable shortly after nucleofection: SB transposase protein was readily detectable one day after transfection with SB100X mRNA (i.e. on day 3 of the manufacturing process, SB-RP cells) (FIG. 11, lane 3).

Importantly, SB transposase protein was not detectable any more at the end of the manufacturing process (i.e. on day 14) (FIG. 12, lane 5). As a positive control, 1 ng of recombinant SB transposase protein was used (lane 1). As a negative control, untransfected T cells sampled on day 3 and on day 14 were used (lane 2 and 4).

Collectively, the data show that SB transposase protein is detectable early after transfection of T cells with SB100X-encoded mRNA; however, SB transposase protein is not detected at the end of the manufacturing process in the DP.

### SUMMARY

First, there is no residual SB transposase detectable in the DP. Because human T cells do not express SB transposase endogenously, transposition is confined to a short time window after nucleofection when transposition can occur from the transfected MCs into the T cell genome. At later timepoints, further rounds of transposition cannot occur because of the absence of transposase, which is key for the maintenance of genomic stability in the DP. Second, the average transposon copy number in the DP fall within the range that is considered safe in the context of human T cells. Importantly, vector-driven insertional oncogenesis has never been observed in human T cells after SB transposition, and not even with gammaretroviral and lentiviral vectors. Third, the genomic distribution of the CAR transposon in the DP is close-to-random and lacks a pronounced preference for integration into genes (including oncogenes and tumor suppressors).

These experimental results provided by the present inventors support the use of SLAMF7 CART cells in clinical trials in humans.

### Identification of the SLAMF7 CAR-T Cells

The gene cassette of the SLAMF7 CAR includes an scFv derived from the humanized monoclonal antibody (mAb) huLuc63 Elotuzumab, an IgG4-Fc spacer domain, the transmembrane and intracellular domain of the human costimulatory molecule CD28, an intracellular signaling domain of the human CD3 chain for T cell activation, and an EGFRt sequence (FIG. 1). SLAMF7 CAR-modified T cells can be identified by staining the SLAMF7 CAR with SLAMF7 protein, and by staining the EGFRt marker with anti-EGFR antibody, and subsequent analysis by flow cytometry (FIG. 13).

### Pharmacology Studies

### Selectivity of the DP

The ability of SLAMF7 CAR-T cells to specifically recognize SLAMF7-positive target cells and distinguish them from SLAMF7-negative cells was analyzed and confirmed. To confirm the selectivity of the DP, K562 cells stably transduced with full-length human SLAMF7 (K562 SLAMF7) were used as positive target, while native K562 that do not express SLAMF7 (K562) were used as negative control. The pharmacological studies were additionally performed using SLAMF7-positive myeloma cell lines as target cells.

Both, CD8⁺ SLAMF7 CAR-T cells and CD4⁺ SLAMF7 CAR-T cells exerted specific effector functions against K562 SLAMF7, but not K562 target cells. CD8⁺ SLAMF7 CAR-T cells conferred high level specific cytolytic activity against K562 SLAMF7; produced IFN-γ and Interleukin-2; and proliferated after stimulation with K562 SLAMF7 cells in co-culture assays. CD4⁺ SLAMF7 CAR-T cells produced IFN-γ and Interleukin-2; and proliferated after stimulation with K562 SLAMF7 cells in co-culture assays.

In contrast, no reactivity was observed after stimulation of CD4⁺ and CD8⁺ SLAMF7 CAR-T cells with the SLAMF7-negative K562 target cell line (FIG. 14 to FIG. 23).

### Cytotoxic and Cytolytic Activity Against Antigen-Positive Target Cell Lines

The cytotoxic/cytoloytic activity of the DP was confirmed in a 2-hour europium release assay, by incubating DP cells at different ratios with SLAMF7-positive or SLAMF7-negative target cells.

Furthermore, the cytotoxic/cytoloytic activity of CD4⁺ and CD8⁺ SLAMF7 CAR-T cells was analyzed in-depth by bioluminescence-based assays using firefly luciferase-expressing target cells. SLAMF7 CAR-T cells or control T cells were incubated at different ratios with SLAMF7-positive (K562 SLAMF7, MM.1S, OPM-2) or SLAMF7-negative target cells (K562). The lysis of target cells was analyzed by measuring their luminescence at different time points. The specificity of the lysis was calculated by offsetting the values achieved with SLAMF7 CAR-T cell to the control T cell values.

The cytotoxic/cytolytic activity of a healthy donor DP was analyzed after 2 hours in a europium release assay (FIG. 14). The cytotoxic/cytolytic activity of another healthy donor DP was analyzed after 4 and 24 hours in a bioluminescence-based assay (FIG. 15).

Antigen-specific lysis by CD8⁺ SB-RP cells of four different healthy donors was tested by bioluminescence-based assay after 4 and 24 hours of coincubation (FIG. 16).

The cytotoxic/cyotolytic capacity of CD4⁺ and CD8⁺ LV-RP cells of four different healthy donors were tested by bioluminescence-based assay after 4 and 24 hours of coincubation (FIG. 17, [5]).

In summary, CD8⁺ as well as CD4⁺ SLAMF7 CAR-T cells are both able to specifically eradicate target cells, which express the SLAMF7 antigen. In contrast, antigen-negative target cells remain unaffected. Consistent with their known ‘intrinsic’ function, target cell eradication by CD8⁺ T cells (“killer” T cells) occurs faster compared to target cell elimination by CD4⁺ T cells (“helper” T cells; FIG. 17). The DP, which consists of a mixture of CD4⁺ and CD8⁺, CAR-negative and CAR-positive T cells, is also efficient in eradicating SLAMF7-positive target cells (FIG. 14, FIG. 15).

Cytokine Release after Antigen-Specific Stimulation

The capacity of CD4⁺ and CD8⁺ SLAMF7 CAR-T cells to antigen-specifically produce and release cytokines was analyzed by Interferon-y and Interleukin-2 ELISA. SLAMF7 CAR-T cells or control T cells were incubated with SLAMF7-positive (K562 SLAMF7, MM.1S, OPM-2, NCI-H929) or SLAMF7-negative target cells (K562) for 20 hours. Cytokine release was measured in the supernatants. As a positive control, T cells were stimulated with phorbol 12-myristate 13-acetate (PMA)/lonomycin; as negative control, they were left untreated and unstimulated.

Cells from a healthy donor DP were co-cultured with target cells for 20 hours at an effector to target cell ratio of 2:1. Secreted Interferon gamma and Interleukin-2 were measured in cell supernatants by ELISA (FIG. 18).

CD4⁺ and CD8⁺ SP-RP cells of four different healthy donors were separately tested for cytokine release after antigen-specific stimulation (FIG. 19).

Cytokine release of CD4⁺ and CD8⁺ LV-RP cells of four different healthy donors was analysed in the supernatants obtained after a 20-hour coculture with target cells (FIG. 20, [5]).

### Conclusion

DP cells secret Interleukin-2 and Interferon-y after stimulation with SLAMF7-positive target cells, but not after stimulation with SLAMF7-negative targets (FIG. 18). CD4⁺ as well as CD8⁺ cells are able to produce these cytokines, however, CD4⁺ cells secrete higher amounts of Interleukin-2 (FIG. 19).

Proliferation after Antigen-Specific Stimulation

The ability of SLAMF7 CAR-T cells to proliferate and expand upon recognizing their respective antigen was explored in a CFSE-based proliferation assay. For the assay, effector T cells were CFSE-labeled and cocultured with SLAMF7-negative or SLAMF7-positive irradiated target cells for 3 days without adding exogenous cytokines. Proliferation was determined by measuring the dilution of the CFSE dye in T cells by flow cytometry.

Cells from a healthy donor DP were labeled with CFSE and co-cultured with irradiated target cells for 3 days. For analysis of proliferation, cells were antibody stained for CD4 and CD8 and both T cell types were analysed separately by gating (FIG. 21).

SB-RP cells were derived from two different healthy donors. CFSE-labeled CD4⁺ and CD8⁺ SB-RP cells were stimulated separately with irradiated target cells for three days (FIG. 22).

CD4⁺ and CD8⁺ LV-RP cells of four different healthy donors were tested for their proliferation capacity (FIG. 23, [5]).

### Conclusion

CD4⁺ and CD8⁺ SLAMF7 CAR-T cells are able to proliferate after stimulation with SLAMF7-positive target cells. Stimulation with SLAMF7-negative target cells (K562) does not induce proliferation. In the absence of an antigen-specific stimulus, there is no proliferation of SLAMF7 CAR-T cells.

### Tumor Eradication in MM.1S/NSG Mice

To test the in vivo functionality, biodistribution and kinetic of the DP, a xenograft mouse model was chosen. In this model, immunodeficient NSG (NOD-SCID-gamma chain k.o.) mice are injected with the myeloma cell line MM.1S. Mice subsequently develop disseminated, systemic MM with medullar and extramedullary manifestations, similar to the clinical situation in newly diagnosed and relapsed/refractory MM patients. The MM.1S cell line has been stably transduced with a firefly-luciferase transgene to enable quantitative analyses of MM.1S distribution and tumor burden by bioluminescence imaging.

After tumor manifestation, mice are treated with CAR-modified or control unmodified T cells. The in vivo expansion, persistence and distribution of the intravenously infused CAR-T cells is monitored by flow cytometric analysis in peripheral blood, as well as in bone marrow and spleen of sacrificed mice.

Experiment 1— DP from CARAMBA_Val #1:

Female NSG mice (two to five months old) were inoculated by tail vein injection with 3×10⁶ MM.1S cells. The development of systemic myeloma and MM.1S cell distribution was monitored by bioluminescence imaging after intraperitoneal injection of D-luciferin. Within 8 days of MM.1S/ffluc inoculation, all mice developed systemic myeloma (FIG. 24-1, d8).

Mice were then treated with a single dose of the DP containing 5×10⁶ SLAMF7 CAR-T cells (and 3.9×10⁶ unmodified T cells as transfection rate was 56%; n=4). A second mice cohort received a lower DP dosage containing 2.5×10⁶ SLAMF7 CAR-T cells (and 2×10⁶ unmodified T cells; n=2) of the same donor. Two mice were left untreated (n=2) and two mice received unmodified T cells of the same donor (n=2).

All mice that were left untreated or received unmodified T cells presented with rapidly increasing bioluminescence signal, and had to be sacrificed due to deleterious myeloma progression. Notably, unmodified T cells mediated a subtle anti-myeloma effect in this experiment, likely due to unspecific (allo-) reactivity of T cells from this donor against the MM1.S cell line (FIG. 24-1, -2).

On the other hand, a rapid reduction in bioluminescence signal was observed in all of the mice that had been treated with the DP (FIG. 24-1, -2). The anti-myeloma effect occurred rapidly and was evident already within one week after DP administration. The anti-myeloma effect was sustained until the end of the observation period in all of the mice that had received the DP at the 5×10⁶ SLAMF7 CAR-T cell dose. In the treatment group that had received the DP at the 2.5×10⁶ SLAMF7 CAR-T cell dose, one mouse (68-2) had to be sacrificed on day 64 due to increasing extramedullary tumor burden; one mouse (68-1) survived until day 108.

Kaplan-Meier analyses showed a statistically significant survival benefit for the DP cohort (5×10⁶) compared to mice that received unmodified T cells and untreated mice (p<0.05) (FIG. 24-3). Overall, the data demonstrate a potent, dose-dependent anti-myeloma effect of the DP.

Experiment 2— DP from CARAMBA_Val #3:

In a second experiment, female NSG mice (three to four months old) were inoculated by tail vein injection with 2×10⁶ MM.1S cells transduced to express the firefly luciferase. The development of systemic myeloma and distribution was monitored by bioluminescence imaging. Within 14 days of MM.1S inoculation, all mice developed systemic myeloma (FIG. 25-1, d14).

Mice were then treated with a single dose of DP containing 5×10⁶ SLAMF7 CAR-T cells (and 1.9×10⁶ unmodified T cells as transfection rate was 72%, n=3), with unmodified T cells of the same donor (n=2) or were left untreated (n=1).

One week after T cell transfer, a reduction in bioluminescence signal in all of the mice that had been treated with the DP was observed (FIG. 25-1, -2, day 21). The anti-myeloma response was sustained until the end of the observation period (day 83) in two of the three mice treated with the DP. The third mouse of this cohort had to be sacrificed on day 55 after tumor inoculation (=day 41 after T cell transfer) due to extramedullary tumor burden. The mice receiving control T cells or left untreated presented an increasing bioluminescence signal, indicating disease progression. Accordingly, mice that had received control T cells or were left untreated had to be euthanized prematurely due to progressive disease (FIG. 25-3).

Interestingly, the two surviving mice of the DP cohort showed an increase in the bioluminescence signal two months after MM.1S cell inoculation, indicating myeloma relapse had occurred. However, with further follow-up, the bioluminescence signal declined, coincident with an increase in SLAMF7 CAR-T cells in peripheral blood (FIG. 25-2). These data show that after induction of myeloma remission, SLAMF7 CAR-T cells can persist at low levels and re-expand in response to myeloma relapse which is a desired quality in the clinical setting.

In summary, SLAMF7 CAR-T cells confer a specific and potent anti-myeloma effect in a murine xenograft model of advanced, systemic myeloma (NSG/MM.1S). The anti-myeloma effect is consistent (response rate: 100%) and leads to a statistically significant survival benefit compared to controls. There was no evidence for clinical toxicity in any of the experiments that has been performed in the NSG/MM1.S model with DP SB-RP.

### Overview on SB-RP Cells and LV-RP Cells

All core experiments for determining the pharmacodynamic and pharmacokinetic of SLAMF7 CAR-T cells have been performed with DP or SB-RP. At the end of the manufacturing process, CD4⁺ and CD8⁺ LV-RP and SB-RP cells, showed similarly high purity of SLAMF7 CAR-modified T cells, and had acquired a SLAMF7^{-/low} phenotype (FIG. 30).

The results of a cytotoxic assay based on the CD8⁺ LV-RP and CD8⁺ SB-RP showed lysis of antigen-presenting target cells after 4 and 24 hours. The control target cell line (K562) was not eradicated by both SLAMF7 CAR-T cell sets (FIG. 31).

Cytokine release after antigen-specific stimulation was evaluated by Interleukin-2 and Interferon-γ ELISA. Both, CD8⁺ LV-RP and CD8⁺ SB-RP, as well as CD4⁺ LV-RP and CD4⁺ SB-RP secreted cytokines in an antigen-dependent manner, and did not release cytokines after stimulation with SLAMF7-negative K562 cells (FIG. 32).

Comparison of SLAMF7 CAR-T Cells with ‘Conventional’ Anti-MM Drugs

The in vitro functionality of LV-RP cells was compared to the in vitro activity of ‘conventional’ anti-MM drugs based on published data including all clinically relevant classes of anti-MM therapies i.e. the monoclonal antibodies Elotuzumab (Target: SLAMF7) and Isatuximab (Target: CD38), the proteasome inhibitor Bortezomib, the immune-modulatory agent Lenalidomide, the alkylating agent Melphalan; and the histone deacetylase inhibitor Panobinostat. The data are summarized in FIG. 33 and FIG. 34.

The comparison indicates that SLAMF7 CAR-T cells are substantially more potent against MM cell lines and primary MM cells than currently available anti-MM agents. This is well illustrated by the cytotoxic effect against MM.1S. Eradication with SLAMF7 CAR-T was 94% of MM.1S cells within 4 hours (FIG. 33A). For ‘conventional’ drugs eradication was substantially lower i.e.:
Elotuzumab: less than 70% of MM.1S after 72 hours of treatment
Bortezomib—less than 30% of MM.1S after 72 hours of treatment
Lenalidomide—about 40% of MM.1S after 72 hours of treatment
Melphalan—less than 40% of MM.1S after 48 hours of treatment
Panobinostat—less than 20% of MM.1S after 48 hours of treatment

In summary, based on in vitro assays SLAMF7 CAR-T cells appear to be the most potent anti-MM agent in the above panel and accomplish almost complete MM cell eradication.

### Pharmacokinetics

Standard pharmacokinetic studies cannot be conducted with SLAMF7 CAR-T cells because they are a ‘living drug’ that does not decay with predictable half-life like ‘conventional’ drugs. Nevertheless, the pharmacokinetic of (human) SLAMF7 CAR-T cells was analyzed in a murine xenograft model in immunodeficient NSG mice.

Experiment 1— DP from CARAMBA_Val #1 (Also See Section 4.3.4):

Female NSG mice (two to five months old) were inoculated by tail vein injection with 3×10⁶ MM.1S/ffluc cells. After 8 days, mice were treated with a single dose of the DP containing 5×10⁶ SLAMF7 CAR-T cells (and 3.9×10⁶ unmodified T cells as transfection rate was 56%; n=4). Serial analyses were done in peripheral blood on days 4, 8 and 14 after T cell administration and showed on average 0.37%, 0.36% and 0.3% human CD45⁺ cells of living cells (n=4, Table 4).

At the end of the observation period 111 days after DP injection, there were still CD45⁺ human T cells with and without CAR detectable in the peripheral blood. A very low percentage of human T cells was detectable in mice 67-1 and 67-2. Mice 67-3 and 67-4 presented with a higher frequency of human T cells, which comprised of a higher fraction of unmodified T cells and a lower fraction of CAR-modified T cells (Table 4).

TABLE 4
Human T cells and human CAR-expressing T cells (in brackets) in peripheral
blood of MM1.S/NSG mice injected with CARAMBA_Val#1 DP cells.

| | | | 14 days after T | 111 days after T cell |
|---|---|---|---|---|
| | 4 days after T cell | 8 days after T cell | cell | administration (end of |
| | administration | administration | administration | observation period) |
| Mouse | 0.48% (0.23%) | 0.32% (n.d.) | 0.39% (0.1%) | 0.12% (n.d.) |
| 67-1 | | | | |
| Mouse | 0.22% (0.11%) | 0.49% (0.14%) | 0.26% (n.d.) | 0.11% (n.d.) |
| 67-2 | | | | |
| Mouse | 0.46% (0.19%) | 0.31% (n.d.) | 0.24% (n.d.) | 0.67% (0.23%) |
| 67-3 | | | | |
| Mouse | 0.31% (0.13%) | 0.3% (n.d.) | 0.4% (n.d.) | 0.78% (0.35%) |
| 67-4 | | | | |

depicted are % values of 7-AAD⁻ ffluc⁻ CD45⁺ single cells (=human T cells) or 7-AAD⁻ ffluc⁻ CD45⁺ EGFR⁺ single cells (=human CAR-expressing T cells).
n.d. = not clearly detectable (<0.1%)

Experiment 2—DP from CARAMBA_Val #3 (Also See Section 4.3.4):

Female NSG mice (three to four months old) were inoculated by tail vein injection with 2×10⁶ MM.1S/ffluc cells. After 14 days of MM.1S/ffluc inoculation, mice were treated with a single dose of DP containing 5×10⁶ SLAMF7 CAR-T cells (and 1.9×10⁶ unmodified T cells as transfection rate was 72%, n=3 mice).

CAR-T cell persistence was regularly measured in the peripheral blood. Mean values of 0.26% and 0.16% of CD45⁺ human T cells were detectable at day 4 and day 7 after T cell injection, respectively. After two weeks, there were almost none human T cells detectable in the peripheral blood (Table 5).

TABLE 5
Human T cells and human CAR-expressing T cells (in brackets) in peripheral
blood of MM1.S/NSG mice injected with CARAMBA_Val#3 DP cells.

| | | | | 69 days after T cell |
|---|---|---|---|---|
| | | | 14 days after | administration (end |
| | 4 days after T cell | 7 days after T cell | T cell | of observation |
| | administration | administration | administration | period) |
| Mouse | 0.29% (0.08%) | n.d. (n.d.) | n.d. (n.d.) | 0.1% (n.d.) |
| 81-1 | | | | |
| Mouse | 0.22% (0.06%) | 0.28% (n.d.) | n.d. (n.d.) | † |
| 81-2 | | | | |
| Mouse | 0.27% (0.1%) | 0.12% (n.d.) | 0.1% (n.d.) | 0.1% (n.d.) |
| 81-3 | | | | |

Depicted are % values of 7-AAD⁻ ffluc⁻ CD45⁺ single cells (=human T cells) or 7-AAD⁻ ffluc⁻ CD45⁺ EGFR⁺ single cells (=human CAR-expressing T cells).
n.d. = not clearly detectable (<0.1%)

Two mice showed an increase in the bioluminescence signal two months after MM.1S cell inoculation, indicating myeloma relapse had occurred (FIG. 25-1, -2). However, with further follow-up, the bioluminescence signal declined, coincident with an increase in SLAMF7 CAR-T cells measured in the peripheral blood. Mouse 81-1 presented a peak level of 1.68% CAR-T cells 62 days after T cell injection (=76 days after MM.1S inoculation), mouse 81-3 presented with 0.41% SLAMF7 CAR-T cells on day 56 after CAR-T cell treatment (=70 days after MM.1S; FIG. 35). The CAR-T cells are therefore re-activated and able to expand in response to tumor relapse. Subsequently, they rapidly re-contract after tumor clearance. At the end of observation period, 69 days after T cell injection only a minute fraction of human T cells could be detected in peripheral blood (Table 5).

Pharmacokinetic data were also derived from EGFRt-sorted and feeder cell expanded SLAMF7 CAR-T cells prepared from healthy donors by SB transposition (SB-DP cells). Unlike the DP, the injected solution was therefore largely free of unmodified “bystander” cells.

Two months old, female NSG mice were inoculated with 2×10⁶ MM1.S and the development of systemic MM was confirmed by bioluminescence imaging. After two weeks, mice were treated with a single dose of 5×10⁶ SLAMF7 CAR-T cells (CD8:CD4 at 1:1 ratio) that was administered by tail vein injection.

For both donors SB-DP cells were administered to eight mice. Results are summarized in Table 6.

### Donor 1:

On day 2 after adoptive transfer, human CD45⁺ T cells comprised on average 0.40% of living cells in peripheral blood (n=8). Bone marrow and spleen were not analyzed on day 2.

On day 6 after adoptive transfer, two mice were sacrificed. In these two mice, human T cells could hardly be detected in peripheral blood but comprised 0.24% of living cells in bone marrow and 0.13% of living cells in spleen.

Two further mice were sacrificed on day 14 after T cell transfer (=day 28 after MM.1S inoculation). In these two mice 0.18% human CD45⁺ T cells were detected in spleen and bone marrow.

Of the four remaining mice, two had to be sacrificed on day 42 and two on day 56 after T cell transfer, due to tumor burden in extramedullary niches. Necropsy was performed on these mice, but only very low levels (<0.05%) of human CD45⁺ cells were detected in peripheral blood, bone marrow and spleen.

Donor 2:

On day 2 after adoptive transfer, human CD45⁺ T cells comprised on average 2.26% of living cells in peripheral blood (n=8). Bone marrow and spleen were not analyzed on day 2.

On day 7 after adoptive transfer, two mice were sacrificed to analyze bone marrow and spleen, peripheral blood was analyzed in all 8 mice. In the two mice that were sacrificed, human CD45⁺ T cells comprised 0.15% of living cells in bone marrow and 0.16% of living cells in spleen. In peripheral blood, 0.79% of living cells were human CD45⁺ T cells.

On day 14 after adoptive transfer, two mice were sacrificed to analyze bone marrow and spleen; peripheral blood was analyzed in all remaining six mice. In the two mice that were sacrificed, SLAMF7 CAR-T cells comprised 0.09% of living cells in bone marrow. In spleen less than 0.05% of living cells were positively stained for human CD45⁺. In peripheral blood, 1.47% of living cells were human CD45⁺ T cells.

In subsequent analyses, low levels of human CD45⁺ cells could be detected in peripheral blood, i.e. 0.05% on day 21, 0.10% on day 25, 0.19% on day 28 after adoptive transfer (n=4).

One mouse had to be sacrificed on day 42, three mice on day 44. There were 0.32% human CD45⁺ cells in peripheral blood and 0.12% in spleen. In bone marrow levels mean CD45 levels were below 0.05% (n=4).

TABLE 6
Average human T cell levels in blood and organs of
MM1.S/NSG mice after injection of 5 × 10⁶ SB-RP cells.

| | | | | | | | Bone | | | |
|---|---|---|---|---|---|---|---|---|---|---|
| | | | | Blood | Bone | Bone | marrow | | | Spleen |
| | Blood | Blood | Blood | d41 or | marrow | marrow | d41 or | Spleen | Spleen | d41 or |
| | d2 | d6/7 | d14 | later | d6/7 | d14 | later | d6/7 | d14 | later |
| Donor 1 | 0.40% | n.d. | n.d. | n.d. | 0.24% | 0.18% | n.d. | 0.13% | 0.18% | n.d. |
| Donor 2 | 2.26% | 0.79% | 1.47% | 0.32% | 0.15% | n.d. | n.d. | 0.16% | n.d. | 0.12% |

Depicted are % values of 7-AAD⁻ ffluc⁻ CD45⁺ single cells (= human T cells), n.d. = not clearly detectable (<0.1%) d = day (after SLAMF7 CAR-T cell administration)

The data show, that following adoptive transfer, SLAMF7 CAR-T cells migrate to lymphoid tissues and can be detected in peripheral blood, bone marrow and spleen after administration. With some donors, the frequency of SLAMF7 CAR-T cells may increase following adoptive transfer, due to antigen-specific stimulation, and subsequently decline again to very low levels.

SLAMF7 CAR T-cells prepared by Sleeping Beauty gene transfer confer superior anti-myeloma efficacy in vivo compared to SLAMF7 CAR T-cells prepared by lentiviral gene transfer.

The anti-myeloma efficacy of SLAMF7 CAR T-cells that had been prepared by Sleeping Beauty gene transfer (SB) according to the present invention was compared to SLAMF7 CAR T-cells prepared by lentiviral gene transfer (LV) in a murine xenograft model (NSG/MM1.S). In this model, the multiple myeloma cell line MM1S is transduced to stably express firefly-luciferase and is engrafted into immunodeficient NSG mice. Mice are inoculated with MM1.S myeloma cells on day 0 by tail vein injection (i.v.) and develop systemic myeloma with manifestations in the bone marrow (medullar lesions) and outside the bone marrow (extra medullar lesions) including manifestations in anatomical niche sites, such as the peritoneum and the injection site next to the tail vein. Subsequently, mice are treated on day 14 with a single dose of SLAMF7 CAR T-cells or non-CAR modified control T cells through tail vein injection (i.v.). The dose of SLAMF7 CAR T-cells is 5×10e6, with CD8+SLAMF7 CAR+ T-cells and CD4+SLAMF7 CAR T-cells formulated at a 1:1 ratio.

The data show that both, SB and LV SLAMF7 CAR T-cells confer an anti-myeloma effect and reduce myeloma burden as evidenced by a decrease in bioluminescence signal within the first 7 days after treatment (i.e. d21 after tumor inoculation). In mice that had been treated with LV SLAMF7 CAR T-cells, we observed an increase of bioluminescence signal after day 21 with re-emerging myeloma manifestations as extramedullary lesions including anatomical niche sites. With further observation, the bioluminescence signal (and hence: myeloma burden) continued to increase in the LV SLAMF7 CAR T-cell treatment group. Analyses in peripheral blood detected no residual LV SLAMF7 CAR T-cells and accordingly, mice were sacrificed at day 56 of the experiment due to relapsing progressive multiple myeloma, in the absence of residual SLAMF7 CAR T-cells. Accordingly, the window of therapeutic activity of LV SLAMF7 CAR T-cells was less than 14 days after adoptive transfer (at 14 days after adoptive transfer mice presented with increasing bioluminescence signal). The survival of mice that had been treated with LV SLAMF7 CAR T-cells was limited to 56 days (after myeloma inoculation), at which time the mice had to be sacrificed due to progressive disease (FIG. 36-1).

In mice that had received the SB SLAMF7 CAR T-cell product, we also observed an increase of bioluminescence signal after day 21 with re-emerging myeloma manifestations as extramedullary lesions including anatomical niche sites. However, in contrast to the LV SLAMF7 CAR T-cell product, the SB SLAMF7 CAR T-cell product was able to control and effectively treat this relapse. Indeed, our analyses in peripheral blood demonstrated the presence of SB SLAMF7 CAR T-cells at low frequency at multiple time points at and after day 21 (FIG. 36-2). Furthermore, SB SLAMF7 CAR T-cells were able to re-expand and eliminate the extramedullar myeloma lesions and induced a second complete remission in the mice. At the analysis time point on day 84 of the experiment, SB SLAMF7 CAR T cells could still be detected in the peripheral blood of mice. At day 126 of the experiment, the mice in this treatment group were myeloma free and alive (FIG. 36-3) and were sacrificed as a planned intervention to terminate the experiment. Accordingly, the window of therapeutic activity for the SB SLAMF7 CAR T-cell product was at least 70 days (i.e. at least 5-fold higher compared to the LV SLAMF7 CAR T-cell product). The survival of mice that had been treated with SB SLAMF7 CAR T-cells was 126 days after myeloma inoculation (i.e. 2.25-fold better compared to mice that had been treated with LV SLAMF7 CAR T-cells.

Collectively, these data demonstrate that SLAMF7 CAR T-cells that are prepared by virus-free SB gene transfer possess greater anti-myeloma efficacy and therapeutic potential, which leads to significantly improved clinical activity, and significantly improved clinical outcome.

### Toxicology

Standard toxicity studies are not feasible with SLAMF7 CAR-T cells and have not been performed.

The safety concerns associated with the administration of the DP are mainly related to undesired side effects of the CAR-T cells, namely the potential of on-target-off-tumor toxicities due to recognition of the target antigen on normal host tissues.

### Species Cross-Reactivity and In Vivo Toxicity Studies

A series of comparative species qualification (binding) studies was conducted to support species selection for toxicology studies. Although the amino acid sequence of the SLAMF7 protein is highly conserved among primate species (human sequence is 98%, 90%, and 89% identical to that of chimpanzee, cynomolgus, and rhesus monkey, respectively), the comprehensive binding analyses surprisingly revealed that SLAMF7 CAR-T cells do not bind SLAMF7 of nonhuman primates or mice (FIG. 37).

Furthermore, the reactivity of LV-RP cells to immobilized, different-species SLAMF7 proteins was analyzed by ELISA. For this purpose 96-well plates were coated with increasing amounts of SLAMF7 molecules of human, mice, chimpanzee, cynomolgus and marmoset monkey. Afterwards, LV-RP cells were incubated on these coated plates and supernatants were analyzed for cytokines. While the incubation with human SLAMF7 led to intense cytokine production (much higher than the 500 pg/ml cytokine maximum standard), there was no antigen-specific cytokine release detectable after incubation with the SLAMF7 molecule of any of the non-human species (FIG. 38). The SLAMF7 CAR therefore is highly specific for the human protein.

The lack of species cross-reactivity (against cynomolgus and rhesus monkey, new zealand white rabbit, CD1 mouse, Sprague dawley rat, beagle dog, yucatan mini-pig) was also shown for the original huLuc63 antibody.

Due to the lack of species-specific cross-reactivity, no relevant animal species or valid transgenic mouse models were identified in which to conduct nonclinical toxicology studies. Given this limitation, the non-clinical safety program consisted primarily of in vitro safety studies utilizing human cells and limited in vivo mouse studies.

Nevertheless, the toxicity and effects on survival of the adoptive transfer of SLAMF7 CAR-T cells to NSG mice could be examined in the course of the four xenograft mice studies.

Specifically, female NSG mice were inoculated intravenously with 2-3×10⁶ human MM.1S/ffluc myeloma cells to provide an antigen-stimulus. 8 to 14 days after MM.1S inoculation, subgroups of mice received up to 5×10⁶ SLAMF7 CAR-T cells derived from healthy donors. Flow cytometry in peripheral blood, bone marrow and spleen showed that SLAMF7 CAR-T cells persisted in mice for more than 4 weeks after adoptive transfer. SLAMF7 CAR-T cells recognized and eliminated MM.1S myeloma cells. Despite this strong in vivo activity of SLAMF7 CAR-T cells, there were no signs of toxicity, in particular no weight loss, no changes in behavior and no premature death. All mice that had been treated with 5×10⁶ SLAMF7 CAR-T cells were alive after a 42-day observation period. Treatment with SLAMF7 CAR-T cells therefore led to a statistically significant survival benefit in all in vivo experiments with DP SB-DP and LV-DP.

### On-Target-Off-Tumor Toxicity

On-target-off-tumor toxicities are due to the undesired recognition by CAR-T cells of the target antigen expressed by normal tissues. Well-known examples are B cell aplasia associated with the administration of CD19-specific CAR-T cells, Kymriah or Yescarta ([18]) in patients with acute B cell leukemia or large B cell lymphoma, respectively. The SLAMF7 antigen is expressed on fractions of normal lymphocytes including NK, NKT, B and T cells. Normal lymphocytes that are SLAMF7^{+/high} are recognized and eliminated by SLAMF7 CAR-T cells. However, in each lymphocyte subset, there is also a SLAMF7^{-/low} fraction that is not recognized and not eliminated by SLAMF7 CAR-T in non-clinical studies ([5]). Therefore, selective elimination of SLAMF7^{+/high} normal lymphocytes may occur in a clinical setting (resulting in lymphoreduction). There is no other know expression of SLAMF7 in normal adult cells or tissues (other than normal lymphocytes) and accordingly, no other on-target-off-tumor toxicity is anticipated.

### SLAMF7 Expression on Healthy Tissues by in Silico Analysis

In silico transcriptomics (GeneSapiens) and gene expression (BioGPS) databases show very high level SLAMF7 expression in MM, high level SLAMF7 expression in normal lymphocytes and no or only extremely low SLAMF7 gene expression in healthy human tissues (FIG. 2, GeneSapiens in silico transcriptomics). Reactivity of SLAMF7 CAR-T cells with other human cells or tissues than lymphocytes is therefore not expected. This is in accordance with the clinical experience gained from the use of the Elotuzumab anti-SLAMF7 mAb from which the targeting domain of the SLAMF7 CAR has been derived.

### SLAMF7 Expression on Normal Lymphocytes by Flow Cytometric Analysis

SLAMF7 expression on normal lymphocyte subpopulations (NK, B cells, CD8⁺ and CD4⁺ naïve and memory T cells, NKT cell, gamma delta T cells, monocytes) was assessed by flow cytometry using an anti-SLAMF7 mAb. Lymphocyte subpopulations were obtained from peripheral blood of MM patients. Overall, the expression level of SLAMF7 on any of the normal lymphocytes subpopulations was lower compared to the expression on malignant plasma cells. Importantly, none of the analyzed normal lymphocyte subpopulations showed a uniform SLAMF7-expression (i.e. expression was bimodal with a positive and negative SLAMF7 fraction; FIG. 39; [5]).

### Recognition of Normal Lymphocytes by SLAMF7 CAR-T Cells

To predict lymphocyte depletion by SLAMF7 CAR-T cells, comprehensive analyses were performed. Most of these fratricide studies were performed with LV-RP cells and are published ([5]). DP cells were cultured with autologous, eFluor-labeled CD8⁺ T cells for 24 hours at a 4:1 effector to target cell ratio. As control, eFluor-labeled CD8⁺ T cells were cultured with unmodified CAR-negative T cells from the same donor. The survival of target cells and their SLAMF7-expression was analyzed by flow cytometry. Gating on eFluor-labeled CD8⁺ T cells revealed an increase in dead (7-AAD-positive) target cells from 18.5% to 35.1% after culturing with DP cells. The remaining target cells had a SLAMF7⁻/′O′″ phenotype (FIG. 40).

In a second experiment CD8⁺ SB-RP cells were cultured with autologous, eFluor-labeled PBMC at a 4:1 effector to target cell ratio. As control, unmodified CD8⁺ T cells were used as effector cells. After 12 hours, lymphocyte subsets were examined by flow cytometry. While CD4⁺ and CD8⁺ T cells remained mostly unaffected, the percentage of viable (7-AAD-negative) NK cells decreased from 92.3% to 68.3%, while viable B cells decreased from 52.9% to 38.8%. The expression of SLAMF7 decreased from 66.5% to 24.8% on NK cells (MFI from 3968 to 1309), from 14.1% to 4.1% on B cells and from 77.2% to 31% on CD8⁺ T cells (MFI from 5943 to 1791) after culturing with SLAMF7 CAR-T cells. The presence of SLAMF7 CAR-T cells therefore affected the composition of PBMC, however, SLAMF7^{-/low} fractions of all tested lymphocyte subsets persisted (FIG. 41).

Furthermore, the killing of normal lymphocytes by SLAMF7 CAR-T cells was intensively analyzed with flow cytometry-based cytotoxic assay. For this experiment, SLAMF7 CAR-T cells were generated using lentiviral gene transfer. The percentage of viable cells was determined using 7ADD staining. Respective lymphocyte subpopulation isolated from peripheral blood of myeloma patients and labeled with eFluor670, were co-cultured with SLAMF7 CAR-T cells or CD19 CAR-T cells (control) for 12 hours. SLAMF7 CAR-T cells induced selective killing of SLAMF7^{+/high} normal lymphocytes, SLAMF7^{-/low} normal lymphocytes were spared from fratricide and remained viable and functional as determined by IFNγ secretion (stimulated by phorbol 12-myristate 13-acetate PMA+ionomycin) that could be elicited immediately at the end of the co-culture assay ([5]).

Taken together, these experiments indicate that NK cell and CD8⁺ T cell levels may be decreased in patients treated with SLAMF7 CAR-T cells, while B cell and CD4⁺ T cells levels might only be slightly decreased. The extent of fratricide may vary between patients, depending on the extent of SLAMF7-expression on normal lymphocyte subsets. SLAMF7^{-/low} lymphocyte subsets are able to survive from fratricide.

TABLE 7
Overview on fratricide assays.

| | DP | SB-RP | LV-RP |
|---|---|---|---|
| CD8⁺ T cells | Depletion of | Depletion of | Depletion of |
| | SLAMF7^{+/high} | SLAMF7^{+/high} | SLAMF7^{+/high} |
| | Survival of | Survival of | Survival of |
| | SLAMF7^{-/low} | SLAMF7^{-/low} | SLAMF7^{-/low} |
| CD4⁺ T cells | not tested | Depletion of | Depletion of |
| | | SLAMF7^{+/high} | SLAMF7^{+/high} |
| | | Survival of | Survival of |
| | | SLAMF7^{-/low} | SLAMF7^{-/low} |
| NK cells | not tested | Depletion of | Depletion of |
| | | SLAMF7^{+/high} | SLAMF7^{+/high} |
| | | Survival of | Survival of |
| | | SLAMF7^{-/low} | SLAMF7^{-/low} |
| B cells | not tested | Depletion of | Depletion of |
| | | SLAMF7^{+/high} | SLAMF7^{+/high} |
| | | Survival of | Survival of |
| | | SLAMF7^{-/low} | SLAMF7^{-/low} |

Functionality of SLAMF7^{low/-} T Cells after Fratricide

It was shown, that SLAMF7 CAR-T cells eradicate SLAMF7^{+/high} lymphocyte subsets, while SLAMF7^{-/low} lymphocytes are spared from fratricide. The functionality of these surviving SLAMF7^{low/neg} T cells was further analyzed. A fraction of virus-specific (here: cytomegalovirus [CMV]-specific) memory T cells was obtained from peripheral blood of healthy donors. These cells expressed SLAMF7, and SLAMF7^{+/high} CMV-specific T cells were eliminated by LV-RP cells. However, the fraction of SLAMF7^{-/low} CMV-specific T cells was spared from fratricide and was still able to respond to stimulation with CMV-antigen.

### Long-Term Consequences of On-Target-Off-Tumor Toxicity

The potential long-term consequences of SLAMF7-mediated fratricide for normal T cells may be extrapolated from the example provided by CD8⁺ and CD4⁺ T cells that were modified to express the SLAMF7-specific CAR. When producing the DP, CD4⁺ T cells and CD8⁺ T cells rapidly acquire a SLAMF7^{-/low} phenotype after transfection with the SLAMF7 CAR gene (FIG. 44). These CAR-positive SLAMF7-negative T cells remain viable and can be expanded within two weeks to therapeutically relevant cell doses. CD8⁺ and CD4⁺ SLAMF7^{-/low} SLAMF7 CAR-modified T cells confer their common cytotoxic and helper functions, indicating that the loss or downregulation of SLAMF7 does not adversely affect T cell survival and function.

### Clinical Consequences of the On-Target-Off-Tumor Effects

The specific fratricide of native SLAMF7^{-/high} normal lymphocytes has implications for the clinical translation of SLAMF7 CAR-T cell therapy. A conceivable side effect of SLAMF7 CAR-T cells is depletion of SLAMF7^{-/high} lymphocytes, a projection that is supported by clinical experience with the anti-SLAMF7 mAb huLuc63 (Elotuzumab), which induces a reduction in lymphocyte counts.

Due to the presumed higher potency of the CAR-T cells as compared to Elotuzumab, a stronger effect on SLAMF7-expressing lymphocytes can be expected in patients than that observed with the antibody. However, the in vitro toxicity studies indicate that a population of SLAMF7^{-/low} lymphocytes survives treatment with the SLAMF7 CAR-T cells. Therefore, complete depletion of normal lymphocytes is not expected. In case of prolonged lymphopenia in the CARAMBA-1 clinical trial, SLAMF7 CAR-T cells may be eliminated using the EGFRt-based suicide mechanism.

CAR-T Cell Depletion with Cetuximab

SLAMF7 CAR-T are equipped with an EGFRt depletion marker. In non-clinical studies in mice, it was demonstrated that administration of the anti-EGFR mAb Cetuximab leads to depletion of CD19 CAR-T cells that co-express the EGFRt marker within few days ([27]). There is only anecdotal experience with using the EGFRt marker in the context of CAR-T cell clinical trials in humans, even though several academic and commercial investigators, routinely include the EGFRt marker into their CAR-T cell DPs.

The mechanisms that leads to CAR-T cell depletion through the EGFRt marker are ADCC and CDC. For ADCC to occur efficiently, Fc-receptor expressing PBMC (e.g. NK cells, monocytes and macrophages) are required. Notably, an anticipated side effect of SLAMF7 CAR-T cells is depletion of SLAMF7^{-/high} PBMC (e.g. SLAMF7^{-/high} NK cells), while SLAMF7^{-/low} PBMC are anticipated to be retained. Therefore, it was tested if SLAMF7^{-/low} PBMC are similarly effective at conferring ADCC as bulk unselected PBMC. PBMC were obtained from healthy donors, and SLAMF7^{+/high} lymphocytes were depleted using immunomagnetic bead selection. Then, ADCC assays were performed using either SLAMF7^{-/low} PBMC or bulk unselected PBMC as effector cells. EGFRt-positive T cells (target cells) were labeled with eFluor670 and then co-cultured with PBMC (effector cells) at an effector to target cell ratio of 20:1 with or without 50 μg/ml Cetuximab (a concentration which is achieved in human serum after i.v. infusion). The data show that in the presence of Cetuximab, the depletion of SLAMF7 CAR-T cells occurred similarly effective with SLAMF7^{-/low} PBMC and bulk unselected PBMC (FIG. 45). Without Cetuximab, no ADCC occurred. Without the EGFRt marker, no ADCC occurred. These data suggest that EGFRt-mediation depletion will still be effective in patients that have been treated with SLAMF7 CAR-T cells. However, because fratricide of SLAMF7^{-/high} lymphocytes will likely lead to lower absolute lymphocyte counts, ADCC may occur with a slower kinetic.

### SUMMARY

The non-clinical development of the SLAMF7 CAR-T cells comprised a thorough investigation of their phenotype, gene integration profile, pharmacodynamic properties as well as examinations on their biodistribution/persistence and toxicity.

SLAMF7 is highly expressed on MM cells; to a lower extent it can also be found on fractions of lymphocyte subsets, especially on CD8⁺ T cells and NK cells.

SLAMF7 CAR-T cells exerted rapid and antigen-specific lysis of a variety of SLAMF7-expressing target cells (SLAMF7⁺ myeloma cell lines OPM-2, NCI-H929, MM.1S, K562 SLAMF7⁺ cells) while leaving non-SLAMF7-expressing cells intact.

Both, SLAMF7 CAR-T cells derived from healthy donors and patient-derived SLAMF7 CAR-T cells, were able to kill SLAMF7⁺ target cell lines and autologous primary myeloma cells.

SLAMF7 CAR-T cells exerted equally potent cytolytic activity against myeloma cells from newly diagnosed and R/R patients.

Generation of SLAMF7 CAR-T cells using SB transposition leads to a safer genomic integration profile.

Based on a comparison with published data, SLAMF7 CAR-T cells eradicated MM cell lines in vitro more potently than approved MM therapies like Elotuzumab, Bortezomib, Lenalidomide, Melphalan and Panobinostat.

In a xenograft model of MM.1S/ffluc in immunodeficient NSG mice, a single dose of SLAMF7 CAR-T cells, (5×10⁶ or 2.5×10⁶ cells) eradicated myeloma cells, while myeloma progression was observed in animals treated with control T cells or were left untreated. Kaplan-Meier analyses showed complete survival of all mice that had been treated with 5×10⁶ SLAMF7 CAR-T cells at the end of the observation period.

Following intravenous injection in mice, SLAMF7 CAR-T cells were primarily detected in blood, spleen and bone marrow. Very low amounts of the CD45⁺ CD4⁺ or CD45⁺ CD8⁺ cells persisted in the mice for several weeks.

Due to the lack of cross-reactivity of human SLAMF7 CAR-T cells with the SLAMF7 molecule of other species, there is no relevant animal species or valid transgenic mouse models to conduct nonclinical toxicology studies.

In mouse pharmacology studies, SLAMF7 CAR-T cells led to a significantly prolonged survival of the animals and a strong anti-MM effect. Relevant body weight losses or other signs of treatment-related toxicity were not observed.

An anticipated toxicity of SLAMF7 CAR-T cell therapy in humans is depletion of SLAMF7^{-/high} normal lymphocytes, a side effect that is known from the clinical use of the anti-SLAMF7 mAb Elotuzumab. However, the fraction of SLAMF7^{-/low} lymphocytes appeared to be spared from fratricide and will preserve the patient's immunocompetence.

SLAMF7 CAR-T cells are equipped with an EGFRt depletion marker as safety switch, that can be triggered by administration of the anti-EGFRt mAb Cetuximab in case of unacceptable toxicity.

In conclusion, SLAMF7 CAR-T cells were adequately characterized and there were no findings, which would preclude the initiation of clinical studies. Side effects known from the use of Elotuzumab will be carefully monitored in the CARAMBA-1 clinical trial with the DP and—due to the higher potency of the SLAMF7 CAR-T cells compared with the antibody—can be expected to occur at higher intensity. However, also greater efficacy can be expected. Furthermore, these side effects are manageable and, if necessary, effects of the SLAMF7 CAR-T cells can be rapidly stopped by activation of their suicide mechanism via administration of the anti-EGFRt mAb Cetuximab. Side effects, which have been reported from other CAR-T cell therapies, will also be closely monitored during the CARAMBA-1 clinical trial.

### Stability

Stability Study to Cover Time from Manufacturing Until Administration to Patients

The stability program was set up to cover short-term (up to 48 h) stability of the final formulated drug product from end of manufacturing during the time needed until administration into the patient. The cells are not frozen, but will be kept at 2-8° C., this was also considered.

A representative batch (GMP validation batch CARAMBA_Val #1) of SLAMF7 CAR-T cells with a cell concentration of 1×10⁶/mL was stored under temperature-controlled conditions at 2-8° C. for up to 48 h. At the beginning and after 24 and 48 h, the following parameters were measured:
Viability of the cells
Percentage of CAR-positive CD4⁺ and CD8⁺ cells, respectively
Percentage of CD34⁺ cells

The results as displayed in Table 8 provide evidence that during storage for 48 h at 2-8° C. both viability as well as the cellular phenotype of SLAMF7 CAR-T cells are maintained.

TABLE 8
Results of short-time stability for batch CARAMBA_Val#1

| | | CAR-positive T cells | |
|---|---|---|---|
| | | [% sum CD45⁺ CD3⁺ (CD4⁺ | |
| Timepoint | Viability | and CD8⁺) EGFRt⁺ cells] | CD34⁺CD45⁺ cells |
| 0 | 96.0% | 57.4% | 0% |
| 24 h | 95.6% | 48.7% | 0% |
| 48 h | 91.1% | 46.2% | 0.01% |

In addition, after 24 h and 48 h the ability of the SLAMF7 CAR-T cells to exhibit biological activity was tested using a qualitative cytotoxicity characterization assay, the Europium release assay as described in section.

In short, after 24 h and 48 h of storage of SLAMF7 CAR-T cell batch CARAMBA_Val #1 at 2-8° C., specific lysis upon co-incubation with SLAMF7-positive (MM.1S ffluc, K562 CS1 ffluc) as well as SLAMF7 negative cells (K562 ffluc, negative control) were measured, using different effector to target cell ratios.

The results are displayed in FIG. 46: both after 24 h and 48 h storage, specific cytotoxic activity can be discerned.

### Supportive Stability Data

A further representative batch (GMP validation batch CARAMBA_Val #3) of SLAMF7 CAR-T cells was subjected to an orthogonal cytotoxicity characterization assay using a bioluminescence assay.

In short, after 1-3 days of storage of SLAMF7 CAR-T cell batch CARAMBA_Val #3 at 2-8° C., specific lysis upon co-incubation with SLAMF7-positive (OPM-2, MM.1S, K562 SLAMF7) as well as SLAMF7-negative cells (K562 CD19) was measured, using different effector to target cell ratios. FIG. 47 displays the read-out after 24-27 h of specific cytotoxicity exerted by SLAMF7 CAR-T cells that were maintained at 2-8° C. for 22, 45 and 66 h, respectively. As can be seen, even after 66 h of storage, the CAR-T cells are still able to perform specific lysis of SLAMF-positive target cells.

The data as obtained from the stability studies confirm that upon storage for up to 48 h, a cell viability of over 90% can be maintained, while the cellular phenotype including the percentage of CAR-positive cells is preserved. In addition, the qualitative characterization of the functional characteristics using two different cytotoxicity assays confirmed that the SLAMF7 CAR-T cell product maintains the ability for specific lysis of SLAMF7-positive cells, even after storage of up to 72 h at 2-8° C.

### Clinical Study Design

An open-label, non-randomized, multicenter clinical trial combines a phase I dose-escalation part with a phase IIa dose-expansion part to assess feasibility, safety and antitumor activity of autologous SLAMF7 CAR-T in patients with MM. The phase I and IIa part will consist of a pre-treatment, treatment, post-treatment phase and long-term follow-up.

Prior to initiation of any study procedures, patients will be provided with the informed consent form (ICF) and undergo screening procedures to determine eligibility. Following confirmation of eligibility, a leukapheresis collection will be performed on each patient to obtain a sufficient quantity of peripheral blood mononuclear cells (PBMCs) for the production of the SLAMF7 CAR-T product.

If necessary, anti-myeloma therapy is allowed in defined periods of time between enrollment and leukapheresis, and between leukapheresis and LD chemotherapy (bridging therapy) for disease control. Baseline evaluations are performed prior to initiation of LD chemotherapy.

Patients will be hospitalized and will receive three days of intravenous infusion of fludarabine (30 mg/m²) and cyclophosphamide (300 mg/m²) for LD chemotherapy starting on Day −5. After the completion of LD chemotherapy and 2 days of rest, fresh (i.e. non-cryopreserved) SLAMF7 CAR-T will be administered as a single dose by intravenous infusion on Day 0. Initially, the interval of SLAMF7 CAR-T infusions between consecutive patients in each cohort will be 28 days. If the SLAMF7 CAR-T infusion is well tolerated and the patient shows no safety concerns the interval will be shortened during the trial.

After infusion with SLAMF7 CAR-T, patients will be followed up for safety and efficacy as inpatients for 12 days and then periodically as outpatients until Month 24 (daily during Month 1, weekly during Month 2, biweekly during Month 3, monthly until Month 12, and quarterly until Month 24). The inpatient interval may be shorter according to the requirements of the national authorities. Each patient's tumor response and disease status will be followed until documented disease progression, death or Month 24. Follow-up visits will include the assessment of SLAMF7 CAR-T pharmacokinetics, efficacy measured using quality of life questionnaires and exploratory endpoints.

A DEC will review the collected data over the course of the trial to evaluate safety, protocol compliance, and scientific validity and integrity of the trial.

Long-term follow-up of SLAMF7 CAR-T-related toxicity as well as disease status, survival status and the treatment with subsequent anti-MM therapies will be done at annual visits (either on site or remotely) for up to 15 years after the last SLAMF7 CAR-T infusion as per regulatory guideline for gene therapy trials.

Retreatment with a second infusion of SLAMF7 CAR-T, after a second cycle of LD chemotherapy, may be considered in the phase IIa part of the trial, if all required selected eligibility criteria are met.

### Dose Escalation—Phase I

Decision of IMP dose-escalation will be based on the recommendations of a DEC. Dose escalation will be performed according to the following scheme.

First, 1 sentinel patient will be treated with SLAMF7 CAR-T at the dose of 3×10⁴ cells/kg body weight. Safety data are collected over a 21-day period (DLT period) after IMP infusion. A DEC will review the patient data and recommend either continuing or stopping dose escalation. The interval of SLAMF7 CAR-T infusions between consecutive patients in each cohort will be 28 days. A DEC review of patient data will be performed for each first patient treated in the first cohort of a dose before treatment of the second patient.

If a DLT occurs in the sentinel patient, then the SLAMF7 CAR-T cell dose will be further decreased to 1×10⁴ cells/kg and evaluated in a cohort of 3 patients. A DEC review will be performed for the first patient treated at this dose before treatment of the second patient.

If no DLT is observed in the sentinel patient, dose-escalation will proceed to the next dose level of 1×10⁵ cells/kg body weight and one cohort of 3 patients will be treated with that dose. A DEC review will be performed for the first patient treated at this dose before treatment of the second patient.

If a DLT occurs in this cohort, dose-escalation will be temporally stopped, and another cohort of 3 patients will be treated with the same dose. If no or no further DLT occurs at that dose level, dose-escalation can continue. If an additional DLT occurs in the cohort of 3 additional patients, dose-escalation will be stopped and the next lower dose level (3×10⁴ cells/kg) will be considered the MTD.

The same approach will be performed in the next higher dose groups (3×10⁵ and 1×10⁶ cells/kg). No dose-escalation will be performed after completion of dose group 1×10⁶ cells/kg. Depending on the safety and toxicity profile of SLAMF7 CAR-T, the study protocol may be amended to continue dose escalation beyond the 1×10⁶ cells/kg dose, and/or to evaluate intermediate dose levels. However, dose escalation beyond the 1×10⁶ cells/kg dose can only be initiated after approval of a respective substantial protocol amendment.

A total of 6 patients will be treated with the MTD.

At the end of phase I, the DEC will review all available patient data and recommend an MTD that shall be used in the subsequent phase IIa part of the clinical trial.

### Dose Expansion—Phase IIa

Patients will be treated with SLAMF7 CAR-T at the MTD defined in phase I. The patients will be sequentially enrolled and treated.

### Treatment Overview

### Lymphodepleting Chemotherapy (Day −5 to Day −3)

The patient will undergo a LD chemotherapy with intravenous cyclophosphamide and intravenous fludarabine.

On Day −5, the following assessments will be performed:

Physical examination and medical history:
Physical examination including:
   Vital signs
   Weight
   Review of organs
   Neurologic examination and status (ICE score)
   Performance status (Karnofsky)
Check of concomitant medication
   Blood analyses:
Blood sampling for:
   Hematology
   Chemistry including
      TLS parameter
      CRS parameter
Coagulation
Serum cytokines (central analysis at UNAV)
Levels of IgG, IgA, IgM, and serum free kappa and gamma light chains
sPEP and sIFE
Flow cytometry for enumeration and phenotyping of peripheral blood T, B, and NK cells, and circulating myeloma cells
   Other assessments:
Recording of PTEAEs
   On Days −4 and −3, the following assessments will be done:
   Physical examination and medical history:
Physical examination including:
   Vital signs
   Weight
   Review of organs
   Neurologic examination and status (ICE score)
   Performance status (Karnofsky)
Check of concomitant medication
   Blood analyses:
Blood sampling for:
   Hematology
   Chemistry including
      TLS parameter
      CRS parameter
Coagulation
   Other assessments:
Recording of PTEAEs
   Break period (Day −2 to Day −1)

During the 2 days between finished LD chemotherapy and the planned administration of the IMP the following assessments will be done:

Physical examination:
including:
   Vital signs
   Weight
   Review of organs
   Neurologic examination and status (ICE score)
   Performance status (Karnofsky)
      Blood analyses:
Blood sampling for:
   Hematology
   Chemistry including
      TLS parameter
      CRS parameter
Coagulation
   Other assessments:
Recording of PTEAEs

### Treatment day (Day 0)

At 4 (+2) hours prior to start of IMP infusion, the following procedures will be done:

Physical examination and medical history:
Physical examination including:
   Vital signs
   Weight
   Review of organs
   Neurologic examination and status (ICE score)
   Performance status (Karnofsky)
Check of concomitant medication
   Blood, urine and bone marrow analyses:
Blood sampling for:
   Hematology
   Chemistry including
      TLS parameter
      CRS parameter
Coagulation
Serum cytokines (central analysis at UNAV)
Levels of IgG, IgA, IgM, and serum free kappa and gamma light chains
sPEP and sIFE
Flow cytometry for enumeration and phenotyping of peripheral blood T, B, and NK cells, and circulating myeloma cells
Viral serology testing for HIV, HBV and HCV. HBV DNA and HCV RNA testing are only required for patients with documented HBV or HCV infection.

### Diagnostics:

12-lead ECG
   Other assessments:
Recording of PTEAEs
   During IMP infusion the following procedures will be done:
Monitoring of vital signs: 15 minutes after the start of the adoptive transfer and at transfer completion, every 15 (±5) minutes in the first hour thereafter and hourly (±10 minutes) for the next 4 hours.

All AEs will be managed by standard medical practice.
Recording of AEs
   At 4 (+2) hours after finishing the IMP infusion the following procedures will be done: Physical examination and medical history:
Physical examination including:
   Vital signs
   Weight
   Review of organs
   Neurologic examination and status (ICE score)
   Performance status (Karnofsky)
Check of concomitant medication
   Blood analyses:
Blood sampling for:
   Hematology
   Chemistry including
      TLS parameter
      CRS parameter
Coagulation
Serum cytokines (central analysis at UNAV)
Flow cytometry for enumeration and phenotyping of peripheral blood T, B, and NK cells, circulating myeloma cells, and SLAMF7 CAR-T cells

### Diagnostics:

12-lead ECG
   Other assessments:
Recording of AEs

### Efficacy Assessments

Response (efficacy) assessments include: serum and urine myeloma paraprotein protein electrophoresis and immunofixation, serum immunoglobulins, serum free light chain assay, serum hematology (for hemoglobin), serum chemistry (for corrected serum calcium and creatinine), clinical and/or radiological extramedullary plasmacytoma assessments (if applicable), radiographic assessment for bone lesions, MRD, and bone marrow aspirate and bone marrow biopsy.

### Assessment of Response

The response after IMP infusion will be assessed monthly until Month 6 and thereafter quarterly until Month 24.

### Laboratory Assessments for Efficacy Parameters

The following ‘routine’ efficacy evaluations of laboratory parameters will be performed locally:
sPEP and uPEP test (performed on 24-hour urine collection) for M-protein measurement. Patients with negative uPEP/sIFE at Baseline will have urine collected in the setting of PD or CR.
sIFE erum and uIFE
Quantitative serum immunoglobulin assessment includes IgG, IgM, and IgA, as well as IgE or IgD only for patients with the respective MM subtype (IgE or IgD)
Quantitative serum FLC (kappa and lambda) with kappa:lambda ratio
Corrected serum calcium, creatinine and hemoglobin will be assessed as part of the safety serum chemistry and hematology assessments, respectively.
   Bone Marrow Aspiration and/or Biopsy

Bone marrow biopsy and/or aspirate will be collected to assess the following parameter:

Percent of myeloma cells to accurately assess response, according to the IMWG Uniform Response Criteria for MM.

MRD status will be assessed by using “next-gen” multiparameter flow cytometry (EuroFlow). By flow cytometry, negative MRD status will be defined at 1 in 10⁵ nucleated cells per IMWG Uniform Response Criteria for MM.

Beside of the protocol specified timepoints for bone marrow biopsy and aspirate, if a patient has resolution of serum and urine M-protein and/or FLC consistent with CR, a bone marrow biopsy and aspirate will be performed to confirm CR. A bone marrow aspirate and biopsy should also be obtained in suspected PD. Bone marrow assessments should include flow cytometry, FISH, cytogenetics, and morphology.

If a bone marrow biopsy or an aspirate is performed at any time during the study, biopsy and/or aspirate samples should be collected for the clinical response assessments, MRD, and for potential research if available. Additional assessments may be performed as part of standard of care as needed for response assessment.

### Bone Lesion Assessment

Bone lesion assessment will be performed locally at Screening and at any time of suspected CR post SLAMF7 CAR-T infusion, and if the treating investigator believes there are signs or symptoms of increased or new skeletal lesions. This assessment can be performed by CT scan, or PET/CT scan provided the same modality will be used for future assessments. All films will be analyzed locally by the site investigator/radiologist. If a bone lesion assessment was performed within 60 days prior to the start of LD chemotherapy, it can be used for the screening assessment.

### Whole-Body Imaging

During the clinical trial, the investigator will assess the disease staging by whole-body imaging preferably with MRI. Alternatively, a CT or PET-CT scan can be used for imaging. The whole-body imaging will include chest, abdomen, and pelvis. If a whole-body imaging was performed within 60 days prior to the start of LD chemotherapy, it can be used for the screening assessment.

### Extramedullary Plasmacytoma Assessments

Extramedullary plasmacytomas (EMP) will be assessed radiographically (PET/CT or MRI) at investigators decision. The radiographic modality used at Screening should be used at each assessment time point throughout the trial (Months 1, 3, 6, 12, and 24). Clinical disease assessment by physical examination will be mandatorily performed at investigators decision for any patient with documented EMP at Screening, Baseline, monthly for 6 months, then every 3 months until Month 24, and at the time of PD/CR.

A tumor biopsy of plasmacytoma should be collected at Screening, only for patients with no measurable disease.

### Quality of Life Outcomes and Health Economics

### Performance Status

The performance status was established to quantify patients' general well-being and activities of daily life. In this trial, patient's performance status will be assessed by the investigator using the Karnofsky grading. The Karnofsky status is a 11-point scale, ranking from 100 (“no complaints”) to 0 scores (“death”).

### EORTC QLQ-C30 and EORTC QLQ-MY20

In this trial, two questionnaires (i.e. EORTC QLQ-C30 and EORTC QLQ-MY20) will be used to assess the patient's health as well as physical, social, emotional, and functional well-being.

The QLQ-C30 is composed of multi-item scales and single item measures. These include five functional scales (physical, role, emotional, cognitive and social), three symptom scales (fatigue, nausea/vomiting, and pain), a global health status/HRQoL scale, and six single items (dyspnea, insomnia, appetite loss, constipation, diarrhea, and financial difficulties). Each of the multi-item scales includes a different set of items—no item occurs in more than one scale.

The QLQ-C30 employs a week recall period for all items and a 4-point scale for the functional and symptom scales/items with response categories “Not at all”, “A little”, “Quite a bit” and “Very much”. The two items assessing global health status/HRQoL utilize a 7-point scale ranging from 1 (“very poor”) to 7 (“excellent”) (Aaronson, 1993).

The QLQ-MY20 is a 20-item myeloma module intended for use among patients varying in disease stage and treatment modality. The module has been validated and shown to be measuring additional aspects of HRQoL, such as body image and future perspective.

Both questionnaires will be completed by the patients at Screening, Baseline, Months 1, 6, 12, and 24 before any clinical assessments are performed at the center. If patients refuse to complete all or any part of a questionnaire, this will be documented. Site personnel should review questionnaires for completeness and ask patients to complete any missing responses.

If the patient withdraws from the study prematurely, all attempts should be made to obtain a final quality-of-life questionnaire prior to patient discontinuation.

### Hospital Resource Utilization

Hospital resource utilization will be assessed based on the numbers of ICU inpatient days, non-ICU inpatient days, outpatient visits and concomitant medication. Dates of admission and discharge to the hospital and to the ICU will be collected together with the reasons for the hospitalization(s).

### Pharmacokinetics and Biomarkers

### Pharmacokinetic of SLAMF7 CAR-T

Pharmacokinetic assessment of CD4+ and CD8+SLAMF7 CAR-T cells will be performed using flow cytometry in peripheral blood and bone marrow

The pharmacokinetic data of CD4+ and CD8+SLAMF7 CAR-T cells will be obtained from individual concentration-time data for peripheral blood and bone marrow by non-compartmental analysis using software SAS Version 9.4 or higher, based on the actual sampling times relative to the referred administration.

### Cytokines and Immune-Related Soluble Factors

### Analyses of Cytokines and Immune-Related Soluble Factors in Serum Will be Performed

### Immunophenotyping

Routine phenotyping analysis in peripheral blood and bone marrow will be performed locally by flow cytometric according to institutional procedures.

Extended phenotyping in peripheral blood and bone marrow will be performed centrally at UNAV. Peripheral blood samples and bone marrow samples for extended immunophenotyping will be collected.

The extended phenotyping will comprise analysis of SLAMF7 CAR-T cells, endogenous immune cells, and myeloma cells.

### Immunogenicity of SLAMF7 CAR-T

The development of a humoral or cellular immune response to SLAMF7 CAR-T will be analysed from peripheral blood. The analyses will be performed centrally at UNAV. Peripheral blood samples will be

### Additional Biomarkers

Additional biomarkers include whole genome sequencing, gene expression profiling, next-generation sequencing and RNA sequencing on SLAMF7 CAR-T cells, endogenous immune cells and myeloma cells. The analyses will be performed at UNAV and UKW. Peripheral blood samples and bone marrow samples will be As novel techniques in genetic analyses evolve rapidly, aliquots of peripheral blood, bone marrow, and/or re-isolated SLAMF7 CAR-T cells, endogenous immune cells and myeloma cells will be cryopreserved and biobanked for future analyses.

### Biostatistical Methods

### General Considerations

The sections below describe statistical methods used in the present application.

### Sample Size Calculation

Sample size calculation has been performed by the lead clinical trial center, UKW, supported by experienced clinical trial statisticians at the Institute of Clinical Epidemiology and Biometry of the University of Würzburg.

No formal sample size calculation is done for the phase I part. A ‘3+3’ design for dose escalation will be used to rapidly define the MTD in small cohorts of patients. The ‘3+3’ design is commonly being used in CAR-T cell clinical trials and proven suitable to rapidly define maximum tolerated dose levels in small cohorts of patients ([31]).

For the phase II component of the trial, the sample size was calculated using both, a 1-stage and Simon's 2-stage design (Minimax), and both calculations estimated the same maximum sample size. However, the power of the 1-stage design (1-beta=0.81) in our case was higher than the power of the 2-stage design (1-beta=0.80), and therefore the 1-stage design was selected. Moreover, a 2-stage design would have mandated stopping the trial if only one out of the initial 15 patients had responded in the first stage which would not be convenient given the poor prognosis of this patient population and the sparse alternative treatment options.

It was therefore decided to apply a 1-stage design which seems a better choice with higher statistical power and lower risk of stopping the trial with only modest amount of information. In a 1-stage design, phase II will include 25 patients (6 patients from phase I with the MTD and 19 additional patients treated at the MTD or at the highest dose level). Assuming that a <10% CR rate can be achieved with standard 3rd line myeloma therapy and that a CR rate of >30% will be of significant interest, the trial would be considered positive if there are ≥6 CRs in 25 patients (80% power, Type I error 0.05). This seems feasible given that we have observed with CD19 CAR-T cells a >90% and 64% CR rate in acute leukemia and lymphoma respectively ([32]).

Analysis Sets and Types of Analyses The following analysis set will be defined:

### Safety Analysis Set (SAF)

The SAF will include all enrolled patients who received one dose of the IMP and will be included in the evaluation of safety and efficacy. If the application of any dose is not certain, the patient will be included in the SAF.

Modified ‘Intended Cell Dose’ Safety Analysis Set (mSAFintent)

The mSAFintent will include patients from the SAF, but exclude the following patients:
treated with a SLAMF7 CAR-T cell product which does not meet the intended cell dose and composition (CD4+: CD8+ T cell ratio of 0.5:1 to 2:1)
whose SLAMF7 CAR-T cell infusion has been delayed

The mSAFintent will be included in the evaluation of safety and efficacy.

Modified ‘other cell dose’ safety analysis set (mSAFother)

The mSAFother will include patients from the SAF, but exclude patients from the mSAFintent. The mSAFother will be included in the evaluation of safety and efficacy.

No formal hypothesis will be stated and statistically tested. All parameters will be descriptively analysed using standard statistical methods.

Tables and graphs, as well as patient listings will be presented by dose groups for the dose escalation part and in general for the dose extension part.

Further details on statistical analyses will be described within the SAP. Deviations from the SAP will be described and justified in the clinical study report.

### Analysis of Study Conduct and Patient Disposition

The number of patients enrolled and receiving one dose of the IMP will be presented in detail to clearly describe the dose escalation pattern used in this trial. The proportion of patients who prematurely discontinued the clinical trial will be summarised together with the reason of discontinuation.

### Dose Limiting Toxicity

The proportion of patients with DLT will be tabulated over time.

### Efficacy Analyses

### Primary Efficacy Variable

For the primary endpoint in phase I, the maximum tolerated dose will be determined and recommended for phase IIa.

For the primary endpoint in phase IIa, the ORR will be calculated according to Kaplan-Meier including 95% confidence intervals at Months 1, 2, 3, 4, 5, 6, 9, 12, 15, 18, 21 and 24 after infusion in the MTD cohort. The median time to response rate will be given as well. A description of response rates will also be given for the remaining dose cohorts.

The IMWG response criteria will be used for assessing the ORR.

Furthermore, in phase I and IIa, the type, frequency and severity of AEs will be tabulated (including SAEs, CRS, and neurotoxicity) as the primary safety endpoint.

### Secondary Variables

For the key secondary endpoint in phase IIa, the CRR will be calculated including 95% confidence intervals at Months 1, 2, 3, 4, 5, 6, 9, 12, 15, 18, 21 and 24 after infusion in the MTD cohort.

The IMWG response criteria will be used for assessing the CRR.

The other secondary endpoints in phase I and IIa will be evaluated as follows:

The percentage of myeloma patients enrolled into the trial who receive ex vivo expanded autologous SLAMF7 CAR-T at Day 0 will be presented using frequency tables.

The time between first SLAMF7 CAR-T infusion and first documentation of response will be analysed using basic statistics. Response is assessed at Months 1, 2, 3, 4, 5, 6, 9, 12, 15, 18, 21 and 24. Additional simmer plots will be presented if appropriate.

The time between first response and PD or death will be analysed using basic statistics. PD and death is assessed at Months 1, 2, 3, 4, 5, 6, 9, 12, 15, 18, 21 and 24. Additional simmer plots will be presented if appropriate.

The time between SLAMF7 CAR-T infusion and first documentation of PD or death will be analysed using basic statistics. PD and death is assessed at Months 1, 2, 3, 4, 5, 6, 9, 12, 15, 18, 21 and 24. Additional simmer plots will be presented if appropriate.

Proportion of MRD evaluable patients will be described using frequency tables. MRD will be assess at Months 1, 3, 6, 12 and 24.

The time between SLAMF7 CAR-T infusion and death will be analysed using basic statistics. Death is assessed at Months 1, 2, 3, 4, 5, 6, 9, 12, 15, 18, 21 and 24. Additional simmer plots will be presented if appropriate.

HRQoL will be assessed at Screening, Baseline, Months 6, 12 and 24 and will be analysed descriptively using basic statistics and frequency tables, as appropriate according to maual.

The PK analysis will be described elsewhere and handles by an external provider.

The exploratory endpoints will be evaluated as follows:

The immunophenotype of SLAMF7 CAR-T and endogenous immune cells will be assessed using basic statistics at Baseline, Months 1, 3, 6, 12 and 24.

The cytokine/chemokine levels in the blood be assessed using basic statistics at Days 0, 1, 3, 7, 10, 14, 21, 28, Week 6, 8, 12, Month 4, 5, 6, 7, 8, 9, 10, 11, 12, 15, 18, 21 and 24.

The humoral and cellular immune response will be analysed using frequency tables at Months 1, 3, 6, 12, and 24.

The change from Baseline in gene expression profile or clonal composition at Months 1, 3, 6, 12 and 24 will be analysed by presenting individual listings.

The kinetic and frequency of SLAMF7 CAR-T after activation of the EGFRt depletion marker in peripheral blood (on Days 0, 1, 3, 7, 10, 14, 21, 28 after administering the first dose of anti-EGFRt antibody [Cetuximab]) and in bone marrow (on Day 28 after administering the first dose of anti-EGFRt antibody [Cetuximab]) will be analysed by showing individual listings of patients receiving EGFRt antibodies.

The hospital resource utilization will be analysed by the number of inpatient ICU days, inpatient non-ICU days, outpatient visits and concomitant medications at Months 6, 12 and 24. Basic statistics and frequency tables will be used.

### Pharmacokinetic Analyses

Levels of circulating CD4+ and CD8+SLAMF7 CAR-T cells will be analyzed descriptively.

### Safety Analyses

For primary and secondary safety endpoints, a descriptive analysis will be performed in each dose cohort in phase I. Data obtained from the clinical centres UKW and OSR will be summarised for each dose cohort (phase I dose-escalation part), and narratives will be used in presentation of the data for safety monitoring by the DEC.

### Adverse Events

AEs will be summarised. Verbatim terms will be mapped to preferred terms and organ systems using the Medical Dictionary for Regulatory Activities (MedDRA). For each preferred term, frequency counts and percentages will be calculated. The nature, severity, seriousness, and relationship to the IMP will be described for all trial patients.

The analysis of the primary safety variable of incidence and severity of toxicities related to the SLAMF7 CAR-T cells will be presented separately.

### Final Analysis

A final analysis will be performed after all patients in the phase I and IIa have completed the Month 24 visit including all efficacy data and the safety data collected up to Month 24. The data base for data up to Month 24 will be closed prior to this analysis. All data collected up to Month 24 will be checked and all queries be resolved before data base closure and analysis. A data review meeting will be conducted before the data base hard lock to check for protocol deviations and to allocate the patients to the analysis sets.

Safety data from the long-term follow-up as well as OS will be analyzed separately.

### Clinical Application

Initially, four clinical-grade SLAMF7 CAR-T cell drug products have been generated from four patients with multiple myeloma. All four drug products met the release criteria, such as sterility, transfection rates, cell viability and lack of hematopoietic stem and progenitor cells (see Table 9).

All four patients were treated with their respective drug product consisting of autologous SLAMF7 CAR-T cells. One patient received 3×10⁴ CAR T cells per kg bodyweight, and three patients received 1×10⁵ SLAMF7 CAR-T cells per kg bodyweight after lymphodepleting preparative chemotherapy (fludarabine/cyclophosphamide day −5 until day −3). The treatment was well tolerated in all patients. Cytokine release syndrome occurred up to grade 1 and no dose limiting toxicities occurred.

### In Vivo Data of Patient D

Patient D was diagnosed with IgG kappa multiple myeloma. Manufacturing of drug product was performed successfully, and the patient was infused with 1×10⁵ CAR-expressing T cells per kg bodyweight.

Despite the relatively low dose of SLAMF7 CAR-T cells that was administered to this patient (Dose level 1 in the Phase I Dose Escalation part of the trial), a clinical efficacy signal was observed.

Specifically, CD8⁺ CAR⁺ T cells were detectable in peripheral blood at a concentration of 1.3% and 3.1% on day 10 and day 14 after SLAMF7 CAR-T cell treatment, respectively (see FIGS. 48, 49). Concomitantly, IL-6 levels increased to more than 30 pg/ml at days 11 and 12, (baseline 2 pg/ml) and IgG values decreased from 2024 to 1646 mg/dl and free kappa light chains decreased from 61.3 to 42.4 mg/I by day 14 after SLAMF7 CAR T-cell treatment (see Table 10). The data support the clinical anti-myeloma activity of SLAMF7 CAR-T cells according to the invention, which can be used advantageously, particularly in human patients.

TABLE 10
Serum parameters of patient D
Serum Parameter

| | Serum | | | Kappa free | Lambda free | Kappa/ |
|---|---|---|---|---|---|---|
| | M-protein | IgG | IgA | light chains | light chains | Lambda |
| Day | (g/dL) | (mg/dl) | (mg/dl) | (mg/l) | (mg/l) | (ratio) |
| −8 | 12.7 | 2107 | 5 | 90.70 | 0.97 | 41.53 |
| 0 | 11.5 | 2024 | 5 | 61.3 | 0.97 | 35.11 |
| 7 | 10.5 | 1774 | 15 | 54.8 | 1.00 | 35.81 |
| 14 | 10.6 | 1646 | 14 | 42.4 | 1.15 | 30.11 |

### INDUSTRIAL APPLICABILITY

The SLAMF7 binding CAR polypeptide, the nucleotide sequence encoding the SLAMF7 binding CAR polypeptide as well as the recombinant immune cell (preferably recombinant lymphocyte, more preferably recombinant T cell) expressing the SLAMF7 binding CAR polypeptide which are used according to the invention, can be industrially manufactured and sold as products for the itemed methods and uses (e.g. for treating a cancer as defined herein), in accordance with known standards for the manufacture of pharmaceutical products. Accordingly, the present invention is industrially applicable.

Sequences

SEQ ID NO: 1 (SLAMF7-binding element; huLuc63 VH, linker, huLuc63 VL)
EVQLVESGGGLVQPGGSLRLSCAASGFDFSRYWMSWVRQAPGKGLEWIGEINPDSSTINY
APSLKDKFIISRDNAKNSLYLQMNSLRAEDTAVYYCARPDGNYWYFDVWGQGTLVTVSSG
GGGSGGGGSGGGGSDIQMTQSPSSLSASVGDRVTITCKASQDVGIAVAWYQQKPGKVPKL
LIYWASTRHTGVPDRFSGSGSGTDFTLTISSLQPEDVATYYCQQYSSYPYTFGQGTKVEIK
SEQ ID NO: 2 (IgG4-FC spacer domain; hinge, CH2, CH3 with 4/2 NQ modification)
ESKYGP PCPPCP APPV AGPSVFLFPP KPKDTLMISRT PEVTCVVVDV SQEDPEVQFN
WYVDGVEVHN AKTKPREEQF QSTYRVVSVL TVLHQDWLNG KEYKCKVSNK GLPSSIEKTI
SKAKGQPREP QVYTLPPSQE EMTKNQVSLT CLVKGFYPSD IAVEWESNGQ PENNYKTTPP
VLDSDGSFFL YSRLTVDKSR WQEGNVFSCS VMHEALHNHY TQKSLSLSLG K
SEQ ID NO: 3 (CD28 transmembrane domain)
MFWVLVWVG GVLACYSLLV TVAFIIFWV
SEQ ID NO: 4 (CD28 cytoplasmic domain)
RSKRSRGGHS DYMNMTPRRP GPTRKHYQPY APPRDFAAYR S
SEQ ID NO: 5 (CD3 zeta domain)
RVKFSRSAD APAYQQGQNQ LYNELNLGRR EEYDVLDKRR GRDPEMGGKP RRKNPQEGLY
NELQKDKMAE AYSEIGMKGE RRRGKGHDGL YQGLSTATKD TYDALHMQAL PPR
SEQ ID NO: 6 (SLAMF7-binding CAR, extracellular domain; huLuc63 VH, linker, huLuc63 VL,
hinge, CH2, CH3)
EVQLVESGGGLVQPGGSLRLSCAASGFDFSRYWMSWVRQAPGKGLEWIGEINPDSSTINY
APSLKDKFIISRDNAKNSLYLQMNSLRAEDTAVYYCARPDGNYWYFDVWGQGTLVTVSSG
GGGSGGGGSGGGGSDIQMTQSPSSLSASVGDRVTITCKASQDVGIAVAWYQQKPGKVPKL
LIYWASTRHTGVPDRFSGSGSGTDFTLTISSLQPEDVATYYCQQYSSYPYTFGQGTKVEI
KESKYGPPCPPCPAPPVAGPSVFLFPPKPKDTLMISRTPEVTCVVVDVSQEDPEVQFNWY
VDGVEVHNAKTKPREEQFQSTYRVVSVLTVLHQDWLNGKEYKCKVSNKGLPSSIEKTISK
AKGQPREPQVYTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGK
SEQ ID NO: 7 (SLAMF7-binding CAR, intracellular signalling domain; CD28 cytoplasmic, CD3
zeta)
RSKRSRGGHSDYMNMTPRRPGPTRKHYQPYAPPRDFAAYRSRVKFSRSADAPAYQQGQNQLYNELNL
GRREEYDVLDKRRGRDPEMGGKPRRKNPQEGLYNELQKDKIVIAEAYSEIGIVIKGERRRGKGHDGLYQGL
STATKDTYDALHMQALPPR
SEQ ID NO: 8 (SLAMF7-binding CAR; huLuc63 VH, linker, huLuc63 VL, hinge, CH2, CH3,
CD28 tm, CD28 cytoplasmic, CD3 zeta)
EVQLVESGGGLVQPGGSLRLSCAASGFDFSRYWMSWVRQAPGKGLEWIGEINPDSSTINY
APSLKDKFIISRDNAKNSLYLQMNSLRAEDTAVYYCARPDGNYWYFDVWGQGTLVTVSSG
GGGSGGGGSGGGGSDIQMTQSPSSLSASVGDRVTITCKASQDVGIAVAWYQQKPGKVPKL
LIYWASTRHTGVPDRFSGSGSGTDFTLTISSLQPEDVATYYCQQYSSYPYTFGQGTKVEI
KESKYGPPCPPCPAPPVAGPSVFLFPPKPKDTLMISRTPEVTCVVVDVSQEDPEVQFNWY
VDGVEVHNAKTKPREEQFQSTYRVVSVLTVLHQDWLNGKEYKCKVSNKGLPSSIEKTISK
AKGQPREPQVYTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGKMFWVLVVVGGV
LACYSLLVTVAFIIFWVRSKRSRGGHSDYMNMTPRRPGPTRKHYQPYAPPRDFAAYRSRV
KFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
SEQ ID NO: 9 (left IR/DR segment; left inverted repeats which are recognized and bound by
transposase)
cagttgaagtcggaagtttacatacacttaagttggagtcattaaaactcgtttttcaactactccacaaatttcttgttaacaaacaat
agttttggcaagtcagttaggacatctactttgtgcatgacacaagtcatttttccaacaattgtttacagacagattatttcacttata
attcactgtatcacaattccagtgggtcagaagtttacatacact
SEQ ID NO: 10 (right IR/DR segment; right inverted repeats which are recognized and bound
by transposase)
agtgtatgtaaacttctgacccactgggaatgtgatgaaagaaataaaagctgaaatgaatcattctctctactattattctgatattt
cacattcttaaaataaagtggtgatcctaactgacctaagacagggaatttttactaggattaaatgtcaggaattgtgaaaaagtga
gtttaaatgtatttggctaaggtgtatgtaaacttccgacttcaactg
SEQ ID NO: 11 (SLAMF7-binding CAR integration cassette comprising left IR/DR, EF-1 alpha
core promoter, Kozak, GMCSF SP, huLuc63 VH, (4GS)3 linker, huLuc63 VL, IgG4 Hinge, IgG4
CH2CH3 NQ, CD28 tm, CD28 cytoplasmic, T2A, EGFRt, right IR/DR)
cagttgaagtcggaagtttacatacacttaagttggagtcattaaaactcgtttttcaactactccacaaatttcttgttaacaaacaat
agttttggcaagtcagttaggacatctactttgtgcatgacacaagtcatttttccaacaattgtttacagacagattatttcacttata
attcactgtatcacaattccagtgggtcagaagtttacatacactaagttgactgtgcctttaaacagcttggaaaattccagaaaatg
atgtcatggctttagaagcttgatatccatggaattcggatctgcgatcgctccggtgcccgtcagtgggcagagcgcacatcgccca
cagtccccgagaagttggggggaggggtcggcaattgaaccggtgcctagagaaggtggcgcggggtaaactgggaaagtgatgt
cgtgtactggctccgcctttttcccgagggtgggggagaaccgtatataagtgcagtagtcgccgtgaacgttctttttcgcaacgggt
ttgccgccagaacacagctgaagcttcgaggggctcgcatctctccttcacgcgcccgccgccctacctgaggccgccatccacgcc
ggttgagtcgcgttctgccgcctcccgcctgtggtgcctcctgaactgcgtccgccgtctaggtaagtttaaagctcaggtcgagaccg
ggcctttgtccggcgctcccttggagcctacctagactcagccggctctccacgctttgcctgaccctgcttgctcaactctacgtctttg
tttcgttttctgttctgcgccgttacagatccaagctgtgaccggcgcctacggctagcgccgccaccatgctgctgctcgtgacatctc
tgctgctgtgcgagctgccccaccccgcctttctgctgattcctgaggtgcagctggtggaaagcggcggaggactggtgcagcctgg
cggatctctgagactgagctgtgccgccagcggcttcgacttcagccggtactggatgagctgggtgcgccaggcccctggcaaagg
cctggaatggatcggcgagatcaaccccgacagcagcaccatcaactacgcccccagcctgaaggacaagttcatcatcagccggg
acaacgccaagaacagcctgtacctgcagatgaactccctgcgggccgaggacaccgccgtgtactattgcgccagacccgacggc
aactactggtacttcgacgtgtggggccagggcaccctcgtgacagtgtctagcggaggcggcggatctggcggagggggatctgg
gggcggaggctctgatatccagatgacccagagccccagcagcctgtctgccagcgtgggcgacagagtgaccatcacatgcaagg
ccagccaggacgtgggaatcgccgtggcctggtatcagcagaaacccggcaaggtgcccaagctgctgatctactgggccagcacc
agacacaccggcgtgcccgatagattttccggcagcggctccggcaccgacttcaccctgacaatcagctccctgcagcctgaggac
gtggccacctactactgccagcagtacagcagctacccctacaccttcggacagggcaccaaggtggaaatcaaagagtctaagta
cggaccgccttgtcctccttgtccagctcctcctgtggccggacctagcgtgttcctgttccccccaaagcccaaggacaccctgatga
tcagccggacccccgaagtgacctgcgtggtggtggatgtgtcccaggaagatcccgaggtgcagttcaattggtacgtggacggcg
tggaagtgcacaacgccaagaccaagcccagagaggaacagttccagagcacctaccgggtggtgtccgtgctgacagtgctgca
ccaggactggctgaacggcaaagagtacaagtgcaaggtgtccaacaagggcctgcccagcagcatcgagaaaaccatcagcaa
ggccaagggccagcctcgcgagccccaggtgtacacactgcctccaagccaggaagagatgaccaagaaccaggtgtccctgacc
tgtctcgtgaagggcttctaccccagcgacattgccgtggaatgggagagcaacggccagcccgagaacaactacaagaccacccc
ccctgtgctggacagcgacggctcattcttcctgtacagcagactgaccgtggacaagagccggtggcaggaaggcaacgtgttcag
ctgcagcgtgatgcacgaggccctgcacaaccactacacccagaagtccctgtctctgagcctgggcaagatgttctgggtgctggt
ggtcgtgggcggagtgctggcctgttacagcctgctcgtgaccgtggccttcatcatcttttgggtgcgcagcaagcggagcagaggc
ggccacagcgactacatgaacatgacccccagacggcctggccccaccagaaagcactaccagccttacgcccctcccagagactt
cgccgcctaccggtccagagtgaagttcagcagaagcgccgacgcccctgcctatcagcagggccagaaccagctgtacaacgag
ctgaacctgggcagacgggaagagtacgacgtgctggataagcggagaggccgggaccctgagatgggcggcaagcctagaaga
aagaacccccaggaaggcctgtataacgaactgcagaaagacaagatggccgaggcctacagcgagatcggcatgaagggcgaa
agaaggcggggcaagggccacgatggcctgtatcagggactgagcaccgccaccaaggatacctatgacgcactgcacatgcagg
ccctgccccccagactcgagggcggaggcgaaggcagaggatctctgctgacatgcggcgacgtggaagagaacccaggccccag
aatgctgctgctcgtgacaagcctgctgctgtgcgagctgccccaccctgcctttctgctgatcccccggaaagtgtgcaacggcatc
ggcatcggagagttcaaggacagcctgtccatcaacgccaccaacatcaagcacttcaagaattgcaccagcatcagcggcgacct
gcacatcctgccagtggcctttagaggcgacagcttcacccacacccccccactggatccacaggaactggatattctgaaaaccgt
aaaggaaatcacagggtttttgctgattcaggcttggcctgaaaacaggacggacctccatgcctttgagaacctagaaatcatacg
cggcaggaccaagcaacatggtcagttttctcttgcagtcgtcagcctgaacataacatccttgggattacgctccctcaaggagata
agtgatggagatgtgataatttcaggaaacaaaaatttgtgctatgcaaatacaataaactggaaaaaactgtttgggacctccggt
cagaaaaccaaaattataagcaacagaggtgaaaacagctgcaaggccacaggccaggtctgccatgccttgtgctcccccgagg
gctgctggggcccggagcccagggactgcgtctcttgccggaatgtcagccgaggcagggaatgcgtggacaagtgcaaccttctg
gagggtgagccaagggagtttgtggagaactctgagtgcatacagtgccacccagagtgcctgcctcaggccatgaacatcacctg
cacaggacggggaccagacaactgtatccagtgtgcccactacattgacggcccccactgcgtcaagacctgcccggcaggagtca
tgggagaaaacaacaccctggtctggaagtacgcagacgccggccatgtgtgccacctgtgccatccaaactgcacctacggatgc
actgggccaggtcttgaaggctgtccaacgaatgggcctaagatcccgtccatcgccactgggatggtgggggccctcctcttgctgc
tggtggtggccctggggatcggcctcttcatgtgagcggccgctctagatggccagatctagcttgtggaaggctactcgaaatgtttg
acccaagttaaacaatttaaaggcaatgctaccaaatactaattgagtgtatgtaaacttctgacccactgggaatgtgatgaaaga
aataaaagctgaaatgaatcattctctctactattattctgatatttcacattcttaaaataaagtggtgatcctaactgacctaagac
agggaatttttactaggattaaatgtcaggaattgtgaaaaagtgagtttaaatgtatttggctaaggtgtatgtaaacttccgacttc
aactg
SEQ ID NO: 13 (SB transposase)
MGKSKEISQDLRKRIVDLHKSGSSLGAISKRLAVPRSSVQTIVRKYKHHGTTQPSYRSGRRRVLSPRDERTL
VRKVQINPRTTAKDLVKMLEETGTKVSISTVKRVLYRHNLKGHSARKKPLLQNRHKKARLRFATAHGDKD
RTFWRNVLWSDETKIELFGHNDHRYVWRKKGEACKPKNTIPTVKHGGGSIMLWGCFAAGGTGALHKID
GIMDAVQYVDILKQHLKTSVRKLKLGRKWVFQHDNDPKHTSKVVAKWLKDNKVKVLEWPSQSPDLNPI
ENLWAELKKRVRARRPTNLTQLHQLCQEEWAKIHPNYCGKLVEGYPKRLTQVKQFKGNATKY
SEQ ID NO: 14 (pcGlobin2-SB100X plasmid)
GACGGATCGGGAGATCTCCCGATCCCCTATGGTGCACTCTCAGTACAATCTGCTCTGATGCCGCATA
GTTAAGCCAGTATCTGCTCCCTGCTTGTGTGTTGGAGGTCGCTGAGTAGTGCGCGAGCAAAATTTAA
GCTACAACAAGGCAAGGCTTGACCGACAATTGCATGAAGAATCTGCTTAGGGTTAGGCGTTTTGCGC
TGCTTCGCGATGTACGGGCCAGATATACGCGTTGACATTGATTATTGACTAGTTATTAATAGTAATCA
ATTACGGGGTCATTAGTTCATAGCCCATATATGGAGTTCCGCGTTACATAACTTACGGTAAATGGCCC
GCCTGGCTGACCGCCCAACGACCCCCGCCCATTGACGTCAATAATGACGTATGTTCCCATAGTAACGC
CAATAGGGACTTTCCATTGACGTCAATGGGTGGAGTATTTACGGTAAACTGCCCACTTGGCAGTACA
TCAAGTGTATCATATGCCAAGTACGCCCCCTATTGACGTCAATGACGGTAAATGGCCCGCCTGGCATT
ATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCC
AAGTCTCCACCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGTCTATATAA
GCAGAGCTCTCTGGCTAACTAGAGAACCCACTGCTTACTGGCTTATCGAAATTAATACGACTCACTAT
AGGGAGACCCAAGCTTGGCACGAGCTGGACTTTAAGCAAGCACCTAAGGACATCCAAAAGCGTAAA
CGGATCCACCATGGGCAAATCCAAGGAGATCTCCCAGGACCTGAGGAAAAGAATCGTGGACCTGCA
CAAATCTGGCTCTTCTCTGGGCGCCATCTCTAAAAGACTGGCCGTGCCTAGGTCCAGCGTGCAGACC
ATTGTGCGGAAATACAAACACCACGGAACCACACAGCCATCTTACCGCTCCGGACGGCGGAGAGTG
CTGTCCCCTAGAGACGAGAGGACCCTCGTGAGAAAAGTGCAGATCAACCCTAGAACAACCGCCAAA
GACCTGGTGAAAATGCTGGAGGAGACCGGCACCAAGGTGTCCATCTCTACCGTGAAGCGCGTGCTG
TACCGGCACAACCTGAAGGGACACTCCGCCCGGAAGAAACCTCTGCTGCAGAATAGGCACAAGAAA
GCCCGGCTGAGATTCGCCACCGCCCACGGCGATAAGGATAGAACCTTTTGGCGCAACGTGCTGTGG
AGCGACGAGACAAAAATCGAGCTGTTCGGGCACAACGATCACCGCTACGTGTGGCGCAAAAAGGGA
GAGGCCTGTAAGCCAAAAAACACCATCCCCACCGTGAAACACGGCGGAGGCTCCATTATGCTGTGG
GGCTGCTTTGCCGCCGGCGGAACCGGAGCCCTGCACAAAATCGACGGCATCATGGATGCCGTGCAG
TACGTGGACATCCTGAAACAGCACCTGAAAACCTCTGTGAGAAAACTGAAACTGGGCCGCAAATGG
GTGTTCCAGCACGACAATGACCCTAAGCACACATCCAAAGTGGTGGCCAAATGGCTGAAAGACAAC
AAAGTGAAAGTGCTGGAGTGGCCTTCCCAGTCCCCCGATCTGAACCCAATTGAGAACCTGTGGGCCG
AGCTGAAGAAAAGAGTGCGGGCCAGACGGCCTACAAACCTGACACAGCTGCACCAGCTGTGTCAGG
AGGAGTGGGCCAAAATCCACCCCAACTACTGTGGCAAGCTGGTGGAGGGATACCCAAAACGGCTGA
CCCAAGTGAAACAGTTCAAGGGCAACGCCACCAAGTACTGAGAATTCTGCAGATATCCATCACACTG
GCGGCCGCTCGAGTCTCATCGCCAATGAACGTCAGGTGTTTTGGAGAAGGTCACACCGCGCGGAGA
TGTTCAAACAAAGATCTAACTCTTTTGCAACAAGATCAATAAAATAATGTTAAAAGCAAAAAAAAAAA
AAAAAAAAAAAAAAAAAAATCTAGAATCGATGCGCGCTAGCCCGGGCCCTATTCTATAGTGTCACCT
AAATGCTAGAGCTCGCTGATCAGCCTCGACTGTGCCTTCTAGTTGCCAGCCATCTGTTGTTTGCCCCTC
CCCCGTGCCTTCCTTGACCCTGGAAGGTGCCACTCCCACTGTCCTTTCCTAATAAAATGAGGAAATTG
CATCGCATTGTCTGAGTAGGTGTCATTCTATTCTGGGGGGTGGGGTGGGGCAGGACAGCAAGGGGG
AGGATTGGGAAGACAATAGCAGGCATGCTGGGGATGCGGTGGGCTCTATGGCTTCTGAGGCGGAA
AGAACCAGCTGGGGCTCTAGGGGGTATCCCCACGCGCCCTGTAGCGGCGCATTAAGCGCGGCGGGT
GTGGTGGTTACGCGCAGCGTGACCGCTACACTTGCCAGCGCCCTAGCGCCCGCTCCCTTCGCTTTCTT
CCCTTCCTTTCTCGCCACGTTCGCCGGCTTTCCCCGTCAAGCTCTAAATCGGGGGCTCCCTTTAGGGTT
CCGATTTAGTGCTTTACGGCACCTCGACCCCAAAAAACTTGATTAGGGTGATGGTTCACGTAGTGGG
CCATCGCCCTGATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATTTTGCCGATT
TCGGCCTATTGGTTAAAAAATGAGCTGATTTAACAAAAATTTAACGCGAATTAATTCTGTGGAATGTG
TGTCAGTTAGGGTGTGGAAAGTCCCCAGGCTCCCCAGCAGGCAGAAGTATGCAAAGCATGCATCTC
AATTAGTCAGCAACCAGGTGTGGAAAGTCCCCAGGCTCCCCAGCAGGCAGAAGTATGCAAAGCATG
CATCTCAATTAGTCAGCAACCATAGTCCCGCCCCTAACTCCGCCCATCCCGCCCCTAACTCCGCCCAGT
TCCGCCCATTCTCCGCCCCATGGCTGACTAATTTTTTTTATTTATGCAGAGGCCGAGGCCGCCTCTGCC
TCTGAGCTATTCCAGAAGTAGTGAGGAGGCTTTTTTGGAGGCCTAGGCTTTTGCAAAAAGCTCCCGG
GAGCTTGTATATCCATTTTGGGATCTGATCAAGAGACAGGATGAGGATGGTTTCGCATGATTGAAGA
AGATGGATTGCACGCAGGTTCTCCGGCCGCTTGGGTGGAGAGGCTATTCGGCTATGACTGGGCACA
ACAGACAATCGGCTGCTCTGATGCCGCCGTGTTGCGGCTGTCAGCGCAGGGGCGCCCGGTCCTTTTT
GTCAAGACCGACCTGTCCGGTGCCCTGAATGAACTGCAGGACGAGGCAGCGCGGCTATCGTGGCTG
GCCACGACGGGCGTTCCTTGCGCAGCTGTGCTCGACGTTGTCACTGAAGCGGGAAGGGACTGGCTG
CTATTGGGCGAAGTGCCGGGGCAGGATCTCCTGTCATCTCACCTTGCTCCTGCCGAGAAAGTATCCA
TCATGGCTGATGCAATGCGGCGGCTGCATACGCTTGATCCGGCTACCTGCCCATTCGACCACCAAGC
GAAACATCGCATCGAGCGAGCACGTACTCGGATGGAAGCCGGTCTTGTCGATCAGGATGATCTGGA
CGAAGAGCATCAGGGGCTCGCGCCAGCCGAACTGTTCGCCAGGCTCAAGGCGCGCATGCCCGACGG
CGAGGATCTCGTCGTGACCCATGGCGATGCCTGCTTGCCGAATATCATGGTGGAAAATGGCCGCTTT
TCTGGATTCATCGACTGTGGCCGGCTGGGTGTGGCGGACCGCTATCAGGACATAGCGTTGGCTACCC
GTGATATTGCTGAAGAGCTTGGCGGCGAATGGGCTGACCGCTTCCTCGTGCTTTACGGTATCGCCGC
TCCCGATTCGCAGCGCATCGCCTTCTATCGCCTTCTTGACGAGTTCTTCTGAGCGGGACTCTGGGGTT
CGAAATGACCGACCAAGCGACGCCCAACCTGCCATCACGAGATTTCGATTCCACCGCCGCCTTCTATG
AAAGGTTGGGCTTCGGAATCGTTTTCCGGGACGCCGGCTGGATGATCCTCCAGCGCGGGGATCTCAT
GCTGGAGTTCTTCGCCCACCCCAACTTGTTTATTGCAGCTTATAATGGTTACAAATAAAGCAATAGCA
TCACAAATTTCACAAATAAAGCATTTTTTTCACTGCATTCTAGTTGTGGTTTGTCCAAACTCATCAATG
TATCTTATCATGTCTGTATACCGTCGACCTCTAGCTAGAGCTTGGCGTAATCATGGTCATAGCTGTTTC
CTGTGTGAAATTGTTATCCGCTCACAATTCCACACAACATACGAGCCGGAAGCATAAAGTGTAAAGC
CTGGGGTGCCTAATGAGTGAGCTAACTCACATTAATTGCGTTGCGCTCACTGCCCGCTTTCCAGTCGG
GAAACCTGTCGTGCCAGCTGCATTAATGAATCGGCCAACGCGCGGGGAGAGGCGGTTTGCGTATTG
GGCGCTCTTCCGCTTCCTCGCTCACTGACTCGCTGCGCTCGGTCGTTCGGCTGCGGCGAGCGGTATCA
GCTCACTCAAAGGCGGTAATACGGTTATCCACAGAATCAGGGGATAACGCAGGAAAGAACATGTGA
GCAAAAGGCCAGCAAAAGGCCAGGAACCGTAAAAAGGCCGCGTTGCTGGCGTTTTTCCATAGGCTC
CGCCCCCCTGACGAGCATCACAAAAATCGACGCTCAAGTCAGAGGTGGCGAAACCCGACAGGACTA
TAAAGATACCAGGCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCACGCTGTAGGTATC
TCAGTTCGGTGTAGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTATCGCCACTGGCAG
CAGCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCTTTTCTACGGGGTC
TGACGCTCAGTGGAACGAAAACTCACGTTAAGGGATTTTGGTCATGAGATTATCAAAAAGGATCTTC
ACCTAGATCCTTTTAAATTAAAAATGAAGTTTTAAATCAATCTAAAGTATATATGAGTAAACTTGGTCT
GACAGTTACCAATGCTTAATCAGTGAGGCACCTATCTCAGCGATCTGTCTATTTCGTTCATCCATAGTT
GCCTGACTCCCCGTCGTGTAGATAACTACGATACGGGAGGGCTTACCATCTGGCCCCAGTGCTGCAA
TGATACCGCGAGACCCACGCTCACCGGCTCCAGATTTATCAGCAATAAACCAGCCAGCCGGAAGGGC
CGAGCGCAGAAGTGGTCCTGCAACTTTATCCGCCTCCATCCAGTCTATTAATTGTTGCCGGGAAGCTA
GAGTAAGTAGTTCGCCAGTTAATAGTTTGCGCAACGTTGTTGCCATTGCTACAGGCATCGTGGTGTC
ACGCTCGTCGTTTGGTATGGCTTCATTCAGCTCCGGTTCCCAACGATCAAGGCGAGTTACATGATCCC
CCATGTTGTGCAAAAAAGCGGTTAGCTCCTTCGGTCCTCCGATCGTTGTCAGAAGTAAGTTGGCCGC
AGTGTTATCACTCATGGTTATGGCAGCACTGCATAATTCTCTTACTGTCATGCCATCCGTAAGATGCTT
TTCTGTGACTGGTGAGTACTCAACCAAGTCATTCTGAGAATAGTGTATGCGGCGACCGAGTTGCTCTT
GCCCGGCGTCAATACGGGATAATACCGCGCCACATAGCAGAACTTTAAAAGTGCTCATCATTGGAAA
ACGTTCTTCGGGGCGAAAACTCTCAAGGATCTTACCGCTGTTGAGATCCAGTTCGATGTAACCCACTC
GTGCACCCAACTGATCTTCAGCATCTTTTACTTTCACCAGCGTTTCTGGGTGAGCAAAAACAGGAAGG
CAAAATGCCGCAAAAAAGGGAATAAGGGCGACACGGAAATGTTgAATACTCATACTCTTCCTTTTTC
AATATTATTGAAGCATTTATCAGGGTTATTGTCTCATGAGCGGATACATATTTGAATGTATTTAGAAA
AATAAACAAATAGGGGTTCCGCGCACATTTCCCCGAAAAGTGCCACCTGACGTC
SEQ ID NO: 15 (EGFRt; truncated EGFR tag to facilitate testing forCAR+ cells and as potential
depletion marker (suicide switch))
R KVCNGIGIGE FKDSLSINAT NIKHFKNCTS ISGDLHILPV AFRGDSFTHT PPLDPQELDI LKTVKEITGF
LLIQAWPENR TDLHAFENLE IIRGRTKQHG QFSLAVVSLN ITSLGLRSLK EISDGDVIIS GNKNLCYANT
INWKKLFGTS GQKTKIISNR GENSCKATGQ VCHALCSPEG CWGPEPRDCV SCRNVSRGRE
CVDKCNLLEG EPREFVENSE CIQCHPECLP QAMNITCTGR GPDNCIQCAH YIDGPHCVKT
CPAGVMGENN TLVWKYADAG HVCHLCHPNC TYGCTGPGLE GCPTNGPKIP SIATGMVGAL
LLLLVVALGI GLFM
SEQ ID NO: 16 (EGFRt; truncated EGFR tag to facilitate testing forCAR+ cells and as potential
depletion marker (suicide switch))
CGGAAAGTGTGCAACGGCATCGGCATCGGAGAGTTCAAGGACAGCCTGTCCATCAACGCCACCAAC
ATCAAGCACTTCAAGAATTGCACCAGCATCAGCGGCGACCTGCACATCCTGCCAGTGGCCTTTAGAG
GCGACAGCTTCACCCACACCCCCCCACTGGATCCACAGGAACTGGATATTCTGAAAACCGTAAAGGA
AATCACAGGGTTTTTGCTGATTCAGGCTTGGCCTGAAAACAGGACGGACCTCCACGCCTTTGAGAAC
CTAGAAATCATACGCGGCAGGACCAAGCAACATGGTCAGTTTTCTCTTGCAGTCGTCAGCCTGAACA
TAACATCCTTGGGATTACGCTCCCTCAAGGAGATAAGTGATGGAGATGTGATAATTTCAGGAAACAA
AAATTTGTGCTATGCAAATACAATAAACTGGAAAAAACTGTTTGGGACCTCCGGTCAGAAAACCAAA
ATTATAAGCAACAGAGGTGAAAACAGCTGCAAGGCCACAGGCCAGGTCTGCCATGCCTTGTGCTCCC
CCGAGGGCTGCTGGGGCCCGGAGCCCAGGGACTGCGTCTCTTGCCGGAATGTCAGCCGAGGCAGG
GAATGCGTGGACAAGTGCAACCTTCTGGAGGGTGAGCCAAGGGAGTTTGTGGAGAACTCTGAGTGC
ATACAGTGCCACCCAGAGTGCCTGCCTCAGGCCATGAACATCACCTGCACAGGACGGGGACCAGAC
AACTGTATCCAGTGTGCCCACTACATTGACGGCCCCCACTGCGTCAAGACCTGCCCGGCAGGAGTCA
TGGGAGAAAACAACACCCTGGTCTGGAAGTACGCAGACGCCGGCCATGTGTGCCACCTGTGCCATC
CAAACTGCACCTACGGATGCACTGGGCCAGGTCTTGAAGGCTGTCCAACGAATGGGCCTAAGATCCC
GTCCATCGCCACTGGGATGGTGGGGGCCCTCCTCTTGCTGCTGGTGGTGGCCCTGGGGATCGGCCTC
TTCATGTGA
SEQ ID NO: 17 (huLuc63 VH: heavy chain of variable fragment derived from SLAMF7-binding
antibody elotuzumab, also known as huLuc63)
EVQLVESG GGLVQPGGSL RLSCAASGFD FSRYWMSWVR QAPGKGLEWI GEINPDSSTI
NYAPSLKDKF IISRDNAKNS LYLQMNSLRA EDTAVYYCAR PDGNYWYFDV WGQGTLVTVS S
SEQ ID NO: 18 (huLuc63 VH: heavy chain of variable fragment derived from SLAMF7-binding
antibody elotuzumab, also known as huLuc63)
GAGGTGCAGCTGGTGGAAAGCGGCGGAGGACTGGTGCAGCCTGGCGGATCTCTGAGACTGAGCTG
TGCCGCCAGCGGCTTCGACTTCAGCCGGTACTGGATGAGCTGGGTGCGCCAGGCCCCTGGCAAAGG
CCTGGAATGGATCGGCGAGATCAACCCCGACAGCAGCACCATCAACTACGCCCCCAGCCTGAAGGA
CAAGTTCATCATCAGCCGGGACAACGCCAAGAACAGCCTGTACCTGCAGATGAACTCCCTGCGGGCC
GAGGACACCGCCGTGTACTATTGCGCCAGACCCGACGGCAACTACTGGTACTTCGACGTGTGGGGC
CAGGGCACCCTCGTGACAGTGTCTAGC
SEQ ID NO: 19 (huLuc63 VL: light chain of variable fragment derived from SLAMF7-binding
antibody elotuzumab)
DIQM TQSPSSLSAS VGDRVTITCK ASQDVGIAVA WYQQKPGKVP KLLIYWASTR HTGVPDRFSG
SGSGTDFTLT ISSLQPEDVA TYYCQQYSS YPYTFGQGTK VEIK
SEQ ID NO: 20 (huLuc63 VL: light chain of variable fragment derived from SLAMF7-binding
antibody elotuzumab)
GATATCCAGATGACCCAGAGCCCCAGCAGCCTGTCTGCCAGCGTGGGCGACAGAGTGACCATCACAT
GCAAGGCCAGCCAGGACGTGGGAATCGCCGTGGCCTGGTATCAGCAGAAACCCGGCAAGGTGCCC
AAGCTGCTGATCTACTGGGCCAGCACCAGACACACCGGCGTGCCCGATAGATTTTCCGGCAGCGGCT
CCGGCACCGACTTCACCCTGACAATCAGCTCCCTGCAGCCTGAGGACGTGGCCACCTACTACTGCCA
GCAGTACAGCAGCTACCCCTACACCTTCGGACAGGGCACCAAGGTGGAAATCAAA
SEQ ID NO: 21 (signal peptide 1)
MLLLVTSLLL CELPHPAFLL IP
SEQ ID NO: 22 (linker)
GGGGSGGGG SGGGGS
SEQ ID NO: 23 (T2A sequence)
LEGGGEG RGSLLTCGDV EENPGPRM
SEQ ID NO: 24 (signal peptide 2)
LL LVTSLLLCEL PHPAFLLIP
SEQ ID NO: 25 (4-1BB domain)
KRGRKKLLYIFKQPFMRPVQTTQEEDGCSCRFPEEEEGGCEL
SEQ ID NO: 26 (4-1BB domain)
aagcggggcagaaagaagctgctgtatatcttcaagcagcccttcatgcggcccgtgcagaccacacaggaagaggacggctgctc
ctgccggttccccgaggaagaagaaggcggctgcgagctg

### REFERENCES

[1] Rajkumar S V, Dimopoulos M A, Palumbo A, Blade J, Merlini G, Mateos M V, et al. International Myeloma Working Group updated criteria for the diagnosis of multiple myeloma. Lancet Oncol 2014; 15(12):e538-48.
[2] Hsi E D, Steinle R, Balasa B, et al. CS1, a potential new therapeutic antibody target for the treatment of multiple myeloma. Clin Cancer Res. 2008; 14(9):2775-84.
[3] Tai Y T, Dillon M, Song W, et al. Anti-CS1 humanized monoclonal antibody HuLuc63 inhibits myeloma cell adhesion and induces antibody-dependent cellular cytotoxicity in the bone marrow milieu. Blood. 2008; 112(4):1329-37.
[4] Dimopoulos M A, Lonial S, White D, Moreau P, Palumbo A, San-Miguel J, Shpilberg O, Anderson K, Grosicki S, Spicka I, Walter-Croneck A, Magen H, Mateos M V, Belch A, Reece D, Beksac M, Bleickardt E, Poulart V, Sheng J, Sy O, Katz J, Singhal A, Richardson P. Elotuzumab plus lenalidomide/dexamethasone for relapsed or refractory multiple myeloma: ELOQUENT-2 follow-up and post-hoc analyses on progression-free survival and tumour growth. Br J Haematol. 2017 September; 178(6):896-905. doi: 10.1111/bjh.14787.
[5] Gogishvili T, Danhof S, Prommersberger S, et al. SLAMF7-CAR T cells eliminate myeloma and confer selective fratricide of SLAMF7+normal lymphocytes. Blood 2017; 130(26):2838-47.
[6] Ocio E M, Vilanova D, Atadja P, et al. In vitro and in vivo rationale for the triple combination of panobinostat (LBH589) and Dexamethasone with either Bortezomib or Lenalidomide in multiple myeloma. Haematologica. 2010; 95(5):794-803.
[7] Sanchez E, Shen J, Steinberg J, et al. The histone deacetylase inhibitor LBH589 enhances the anti-myeloma effects of chemotherapy in vitro and in vivo. Leuk Res. 2011; 35(3):373-9.
[8] Deckert J, Wetzel M C, Bartle L M, et al. SAR650984, A Novel Humanized CD38-Targeting Antibody, Demonstrates Potent Antitumor Activity in Models of Multiple Myeloma and Other CD38+Hematologic Malignancies. Clin Cancer Res. 2014; 20(17):4574-83.
[9] Geurts, A. M., et al., Gene transfer into genomes of transposon system. Mol Ther, 2003. 8(1): p. 108-17.
[10] Maude S L, Barrett D, Teachley D T et al. Managing cytokine release novel T cell-engaging therapies. Cancer J. 2014; 20 (2):119-22.
[11] Lee D W, Santomasso B D, Locke F L Syndrome and Neurologic Toxicity Associated Transplant. 2019; 25(4):625-38.
[12] Teachey D T, Simon F. Lacey, Pamela A. Shaw, et al. Identification of Predictive Biomarkers for Cytokine Release Syndrome after Chimeric Antigen Receptor T-cell Therapy for Acute Lymphoblastic Leukemia. Cancer Discov. 2016 June; 6(6):664-79.
[13] Grupp S A, Kalos M, Barrett D, et al. Chimeric antigen receptor lymphoid leukemia. N Engl J Med. 2013; 368(16):1509-18.
[14] Lee D W, Gardner R, Porter D L, et al. Current concepts in the diagnosis and management of cytokine release syndrome. Blood. 2014 Jul. 10; 124(2):188-95.
[15] Davila M L, Rivieri I, Wang X, et al. Efficacy and toxicity management of 19-28z CAR T cell therapy in B cell acute lymphoblastic leukemia. Sci Transl Med. 2014; 6(224): 224ra25.
[16] Lee D W, Kochenderfer J N, Stetler-Stevenson M, et al. T antigen receptors for acute lymphoblastic leukaemia in children dose-escalation trial. Lancet. 2015; 385(9967):517-28.
[17] Brudno, Jennifer N., and James N. Kochenderfer. “Toxicities of chimeric antigen receptor T cells: recognition and management.” Blood, The Journal of the American Society of Hematology 127.26 (2016): 3321-3330.
[18] Neelapu S S, Tummala S, Kebriaei P et al. Chimeric antigen receptor T-cell therapy assessment and management of toxicities. Nat Rev Clin Oncol. 2018; 15:47-62.
[19] Kochenderfer J N, Dudley M E, Carpenter R O, et al. Donor-derived CD19-targeted T cells cause regression of malignancy persisting after allogeneic hematopoietic stem cell transplantation. Blood. 2013 Dec. 12; 122(25):4129-39.
[20] Lonial S, Dimopoulos M, Palumbo A, et al. Elotuzumab Therapy Multiple Myeloma. N Engl J Med. 2015; 373(7):621-31.
[21] Brentjens R, Yeh R, Bernal Y, et al. Treatment of chronic lymphocytic leukemia with genetically targeted autologous T cells: case report of an unforeseen adverse event in a phase I clinical trial. Mol Ther. 2010; 18(4):666-8.
[22] Recchia A, Mavilio F. Site-specific integration into the human genome: ready for clinical application? Rejuvenation Res. 2006 Winter; 9(4):446-9.
[23] Lupo Stanghellini M, Bonini C, Oliveira G, et al. GvHD kinetics after haploidentical TK-cells:in vivo HSV-TK suicide machinery is effective in GvHD control and provide a long-term immunesuppressive treatment-free survival. Blood. 2014; 124: 548.
[24] Miskey C, Amberger M, Reiser M, et a. Genomic Analyses of SLAMF7 CAR-T Cells Manufactured by Sleeping Beauty Transposon Gene Transfer for Immunotherapy of Multiple Myeloma. bioRxiv. 2019.
[25] Cameron B J, Gerry A B, Dukes J, et al. Identification of a Titin-derived HLA-A1-presented peptide as a cross-reactive target for engineered MAGE A3-directed T cells. Sci Transl Med. 2013; 5(197):197ra103.
[26] Wang X, Chang W C, Wong C W, et al. A transgene-encoded cell surface polypeptide for selection, in vivo tracking, and ablation of engineered cells. Blood. 2011; 118(5):1255-63.
[27] Paszkiewicz Pi, Fräßle SP, Srivastava S, et al. Targeted antibody-mediated depletion of murine CD19 CAR T cells permanently reverses B cell aplasia. J Clin Invest. 2016; 126(11):4262-72.
[28] Gogol-Döring A, Ammar I, Gupta S, et al. Genome-wide Profiling Reveals Remarkable Parallels Between Insertion Site Selection Properties of the MLV Retrovirus and the Transposon in Primary Human CD4(+) T Cells. Mol Ther. 2016; 24(3):592-606.
[29] Monjezi R, Miskey C, Gogishvili T, et al. Enhanced CAR T-cell engineering using non-viral Sleeping Beauty transposition from minicircle vectors. Leukemia. 2017; 31(1):186-194.
[30] Maude S L, Laetsch T W, Buechner J et al. Tisagenlecleucel in Children and Young Adults with B-Cell Lymphoblastic Leukemia. N Engl J Med. 2018 Feb. 1; 378(5):439-448.
[31] Ramos C A, Savoldo B, Dotti G. CD19-CAR Trials. Cancer J 2014; 20(2): 112-118.
[32] Turtle C J, Hanafi L A, Berger C, et al. Immunotherapy of non-Hodgkin's lymphoma with a defined ratio of CD8+ and CD4+CD19-specific chimeric antigen receptor-modified T cells. Sci Transl Med. 2016b; 8(355):355ra116.
[33] Beckert, Bertrand, and Benoît Masquida. “Synthesis of RNA by in vitro transcription.” Rna. Humana Press, 2011. 29-41.
[34] Baronti, Lorenzo, et al. “A guide to large-scale RNA sample preparation.” Analytical and bioanalytical chemistry 410.14 (2018): 3239-3252.
[35] Kochetkov, S. N., E. E. Rusakova, and V. L. Tunitskaya. “Recent studies of T7 RNA polymerase mechanism.” FEBS letters 440.3 (1998): 264-267.
[36] Mayrhofer, Peter, et al. “Minicircle-DNA production by site specific recombination and protein—DNA interaction chromatography.” The Journal of Gene Medicine: A cross-disciplinary journal for research on the science of gene transfer and its clinical applications 10.11 (2008): 1253-1269.
[37] Schleef, Martin, et al. “Minicircle: Next generation DNA vectors for vaccination.” Gene Therapy of Solid Cancers. Humana Press, New York, N.Y., 2015. 327-339.

1. A SLAMF7 binding chimeric antigen receptor (CAR) polypeptide, comprising at least one extracellular ligand binding domain, a transmembrane domain and at least one intracellular signalling domain, wherein said extracellular ligand binding domain comprises a SLAMF7-binding element and an IgG4-FC spacer domain, wherein said transmembrane domain comprises a CD28 transmembrane domain, and wherein said intracellular signalling domain comprises a costimulatory domain and a CD3 zeta domain.

2. The SLAMF7 binding CAR polypeptide according to claim 1, wherein the SLAMF7-binding element is represented by an amino acid sequence shown in SEQ ID NO: 1 or by an amino acid sequence having at least 90% identity to an amino acid sequence shown in SEQ ID NO: 1,
optionally wherein the IgG4-FC spacer domain is represented by an amino acid sequence shown in SEQ ID NO: 2 or by an amino acid sequence having at least 90% identity to an amino acid sequence shown in SEQ ID NO: 2,
wherein the CD28 transmembrane domain is represented by an amino acid sequence shown in SEQ ID NO: 3 or by an amino acid sequence having at least 90% identity to an amino acid sequence shown in SEQ ID NO: 3.

3-4. (canceled)

5. The SLAMF7 binding CAR polypeptide according to claim 1, wherein the costimulatory domain is a CD28 cytoplasmic domain or a 4-1BB costimulatory domain, optionally wherein the costimulatory domain is a CD28 cytoplasmic domain.

6. (canceled)

7. The SLAMF7 binding CAR polypeptide according to claim 1, wherein the CD28 cytoplasmic domain is represented by an amino acid sequence shown in SEQ ID NO: 4 or by an amino acid sequence having at least 90% identity to an amino acid sequence shown in SEQ ID NO: 4, and wherein the 4-1BB costimulatory domain is optionally represented by an amino acid sequence shown in SEQ ID NO: 25 or by an amino acid sequence having at least 90% identity to an amino acid sequence shown in SEQ ID NO: 25, wherein the CD3 zeta domain is optionally represented by an amino acid sequence shown in SEQ ID NO: 5 or by an amino acid sequence having at least 90% identity to an amino acid sequence shown in SEQ ID NO: 5.

8-9. (canceled)

10. The SLAMF7 binding CAR polypeptide according to claim 1, wherein said extracellular domain comprises an amino acid sequence shown in SEQ ID NO: 6 or an amino acid sequence having at least 90% identity to an amino acid sequence shown in SEQ ID NO: 6, said transmembrane domain comprises an amino acid sequence shown in SEQ ID NO: 3 or an amino acid sequence having at least 90% identity to an amino acid sequence shown in SEQ ID NO: 3 and said intracellular signalling domain comprises an amino acid sequence shown in SEQ ID NO: 7 or an amino acid sequence having at least 90% identity to an amino acid sequence shown in SEQ ID NO: 7, optionally, wherein the CAR polypeptide comprises an amino acid sequence shown in SEQ ID NO: 8 or an amino acid sequence having at least 90% identity to an amino acid sequence shown in SEQ ID NO: 8.

11. (canceled)

12. A polynucleotide encoding the SLAMF7-CAR polypeptide according to claim 1.

13. The polynucleotide according to claim 12, wherein the polynucleotide further comprises flanking segments in 5′-direction and in 3′-direction of the polynucleotide encoding the SLAMF7-CAR polypeptide, wherein optionally the flanking segment in 5′-direction is a left inverted repeat/direct repeat (IR/DR) segment and the flanking segment in 3′-direction is a right inverted repeat/direct repeat (IR/DR) segment, optionally, wherein the left IR/DR segment is represented by SEQ ID NO: 9 and right IR/DR segment is represented by SEQ ID NO: 10.

14-15. (canceled)

16. The polynucleotide according to claim 12, wherein the polynucleotide comprises a nucleotide sequence of a left IR/DR, a polynucleotide sequence encoding the SLAMF7-CAR polypeptide and a nucleotide sequence of a right IR/DR, optionally, wherein the polynucleotide comprises a nucleotide sequence represented by SEQ ID NO: 11.

17. (canceled)

18. An expression vector comprising a polynucleotide according to claim 12, optionally wherein the expression vector is a minimal DNA expression cassette.

19. (canceled)

20. The expression vector according to claim 18, wherein expression vector is a transposon donor DNA molecule, or wherein the expression vector is a minicircle DNA, further optionally comprising a polynucleotide sequence shown in SEQ ID NO: 11, optionally, comprising a polynucleotide sequence shown in SEQ ID NO: 12.

21-23. (canceled)

24. A recombinant immune cell comprising a polynucleotide according to claim 12.

25. The recombinant immune cell according to claim 24, wherein the polynucleotide is located in the nuclear genome of the immune cell, optionally, wherein the polynucleotide is expressed, wherein said recombinant immune cell is a recombinant lymphocyte, optionally, wherein said recombinant lymphocyte is a recombinant T cell, wherein said recombinant T cell is a recombinant CD4+cell or a recombinant CD8+cell, optionally further expressing EGFRt, wherein said recombinant immune cell is a recombinant human cell, further optionally, wherein said recombinant immune cell does not comprise an amino acid sequence of the SB transposase as represented by SEQ ID NO: 13 or fragments thereof in a detectable amount at day 14 after gene transfer.

26-32. (canceled)

33. Method for producing recombinant immune cells, comprising the steps of
(a) isolating immune cells from a blood sample of a subject
(b) transforming immune cells using a transposable element comprising a polynucleotide according to claim 12 and a Sleeping Beauty (SB) transposase to produce recombinant immune cells
(c) purifying immune cells.

34. The method according to claim 33, wherein the immune cell is a lymphocyte, optionally, wherein the lymphocyte is a T cell, wherein the T cell is a CD4⁺ cell and/or a CD8⁺ cell, wherein the recombinant CD4⁺ T cells and the recombinant CD8⁺ T cells are optionally expanded separately, further optionally, wherein the subject is a human.

35-38. (canceled)

39. The method according to claim 33, wherein a plurality of recombinant CD4⁺ T cells and a plurality of recombinant CD8⁺ T cells are combined in a defined ratio to form a composition of recombinant T cells, wherein the ratio of said recombinant T cells in the composition is in the range of 0.5:1 to 2:1, optionally, wherein the ratio of the recombinant CD4+ T cells and recombinant CD8+ T cells in the composition is 1:1, optionally, wherein the SB transposase is represented by an amino acid sequence shown in SEQ ID NO: 13, further optionally, wherein the recombinant immune cells do not comprise an amino acid sequence of the SB transposase as represented by SEQ ID NO: 13 or fragments thereof in a detectable amount at day 14 after gene transfer.

40-42. (canceled)

43. A recombinant immune cell obtainable by the method of claim 33.

44. A pharmaceutical composition comprising a plurality of recombinant immune cells according to claim 24.

45. A pharmaceutical composition according to claim 44 for use as a medicament.

46. A pharmaceutical composition according to claim 44 for use in a method of treating cancer, wherein in the method the pharmaceutical composition is to be administered to a subject, wherein, optionally, the pharmaceutical composition to be administered comprises recombinant immune cells in a dose of about 1×10⁴ cells/kg body weight, of about 3×10⁴ cells/kg body weight, of about 1×10⁵ cells/kg body weight, of about 3×10⁵ cells/kg body weight, of about 1×10⁶ cells/kg body weight, of about 3×10⁶ cells/kg body weight, of about 1×10⁷ cells/kg body weight, of about 3×10⁷ cells/kg body weight, of about 1×10⁸ cells/kg body weight, of about 3×10⁸ cells/kg body weight, of about 1×10⁹ cells/kg body weight, or of about 3×10⁹ cells/kg body weight.

47. (canceled)

48. The pharmaceutical composition for use according to claim 45, wherein the pharmaceutical composition is to be administered intravenously, optionally, wherein the recombinant immune cells are to be administered in a single dose, or in multiple doses.

49-50. (canceled)

51. The pharmaceutical composition for use according to claim 45, wherein said recombinant immune cells are recombinant lymphocytes, optionally, wherein said recombinant lymphocytes are recombinant T cells, wherein said recombinant T cells are CD4⁺ T cells and/or CD8⁺ T cells, further optionally wherein said recombinant T cells are present in a defined ratio, wherein said ratio is in a range of 0.5:1 to 2:1, wherein said ratio is about 1:1, wherein said subject is optionally a human, wherein said cancer is optionally multiple myeloma.

52-58. (canceled)
